(19) United States
(12) Patent Application Publication
(10) Pub. No.: US 2024/0082368 A1
(43) Pub. Date: Mar. 14, 2024

(54) Treatment of Brain Damage

(71) Applicant: Ipsen Biopham Limited, Wrexham (GB)

(72) Inventor: Mikhail KALINICHEV, Wrexham (GB)

(21) Appl. No.: 18/261,402

(22) PCT Filed: Jan. 14, 2022
(86) PCT No.: PCT/GB2022/050077

§ 371 Date: Jul. 13, 2023

(30) Foreign Application Priority Data
Jan. 15, 2021 (GB) 2100566.5

(51) Int. Cl. A61K 38/48

(52) U.S. Cl. CPC A61K 38/4893; C12Y 304/24069

(57) ABSTRACT

The present invention provides a clostridial neurotoxin for use in a method of treating a brain disorder in a patient by promoting a neuroimmune response.

### FIELD OF THE INVENTION

The present invention relates to the treatment of brain disorders, more particularly by promoting activity of a neuroimmune response at a site of brain tissue damage.

### BACKGROUND

Brain disorders typically result from damage or injury to neural tissue, and such damage can be a principal cause of the symptoms underlying or defining a neurological disorder. By way of example, when a patient suffers a stroke, a blood vessel in the brain becomes blocked or bursts (e.g. hemorrhages). Thus, nerve cells in the brain tissue, depleted of oxygen, which would otherwise be provided by a healthy blood supply, become injured or die, impairing nerve cell activity. Such lack of nerve activity can lead to symptoms including muscle weakness, paralysis, lost sensation on one side of the body, difficulty speaking, confusion, problems with vision, dizziness, loss of balance and coordination, and, in some hemorrhagic strokes, a sudden, severe headache.

In a further example, infectious disease of the brain can lead to cell death underlying brain tissue damage. In bacterial meningitis, bacteria such as Streptococcus pneumoniae can invade the meninges (membranes that help protect brain and spinal cord tissue from injury), with subsequent damage to the meninges resulting in nerve cell death. If a virus reaches and affects the brain, nerve tissue damage can be caused by virus lytic effects on brain cells (e.g. in the case of cytomegalovirus), induced apoptosis (e.g. in the case of vesicular stomatitis virus, VSV), or secondary damage due to release of glutamate, DNA, and other inducers of brain damage.

In a patient suffering brain cancer, tumors can cause physical damage to nerve tissue in the brain by pressing against and applying pressure to nerve cells. Damage can also be caused by tumor movement in metastatic cases.

While there exist treatment options to alleviate patient symptoms arising from such nerve damage, comparatively few of these function in a way that promote repair (else facilitates said repair) of the actual damage to the nerve tissue. For example, in stroke, the only therapeutic options are local recanalization and systemic thrombolysis (which aim to restore blood flow to mitigate nerve damage). However, such therapeutic options are not suitable for all patients, and/or may cause yet further damage.

The present invention solves one or more of the above-mentioned problems.

### SUMMARY OF THE INVENTION

In more detail, the present invention is predicated upon the surprising finding that a clostridial neurotoxin, when administered to a patient suffering brain disorder-induced damage to tissue of the nervous system, promotes immune cell (e.g. neuroimmune cell) activity in the area of neural tissue damage. In other words, a clostridial neurotoxin may surprisingly by used to promote neuroimmune activity as a means to prevent/repair damage to neural tissue in a patient suffering a brain disorder.

More particularly, it is demonstrated herein that the activity of microglia (located in the vicinity of an area of brain tissue damage) is promoted in a patient that has received a dose of clostridial neurotoxin. Microglia are key macrophages of the brain involved in clearing damaged nerve tissue and foreign bodies. This represents a surprising utility of clostridial neurotoxins, which are introduced below.

Bacteria in the genus Clostridia produce highly potent and specific protein toxins, which can (temporarily) impair function of neurons and other cells to which they are delivered. Examples of such clostridial toxins include the neurotoxins produced by C. tetani (TeNT) and by C. botulinum (BoNT) serotypes A-G, and X (see WO 2018/009903 A2), as well as those produced by C. baratii and C. butyricum.

Among the clostridial neurotoxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Clostridial neurotoxins provide non-cytotoxic protease activity. Non-cytotoxic proteases act by proteolytically cleaving intracellular transport proteins known as SNARE proteins (e.g. SNAP-25, VAMP, or Syntaxin). The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

In view of the ubiquitous nature of SNARE proteins, clostridial neurotoxins such as botulinum toxin have been successfully employed in a wide range of therapies. The inventors have demonstrated a surprising utility of clostridial neurotoxins in treating brain disorders by promoting neuroimmune activity.

A common feature of brain disorders is that the host's own neuroimmune response reacts to insults underlying the brain disorder by clearing and/or preventing brain tissue damage. In the case of infectious disease, there may be (for example) an innate response in which macrophages (e.g. microglia) migrate to a site of infection and phagocytose the invading pathogen. Macrophages are a type of white blood cell of the immune system that engulf and digest cellular debris, foreign substances, microbes, cancer cells, and other non-host agents, in a process called phagocytosis.

When neuronal cell death occurs, microglia react by migrating to the lesion (damage) site, where they phagocytose cellular debris. Thus, microglia can be reactive to brain disorders by removing brain tissue caused by physical damage (e.g. brain tumors). For example, microglial phagocytosis may clear dead cells, which might otherwise release noxious substances into their environment and thereby exacerbate tissue damage. It has also been reported that microglia play a role in the defense against brain tumors directly. Furthermore, microglia have been reported to be involved in ‘synaptic pruning’ in which they engulf synaptic material helping to prevent synaptic abnormalities seen in some neurodevelopmental disorders.

Thus, by promoting neuroimmune activity in response to neural tissue damage, clostridial neurotoxin administration advantageously finds utility in treating brain disorders.

### DETAILED DESCRIPTION

Broad aspects of the invention provide any of the following:
a clostridial neurotoxin for use in a method of treating a brain disorder;
a method of treating a brain disorder in a patient, the method comprising administering a clostridial neurotoxin to the patient;
use of a clostridial neurotoxin in the manufacture of a medicament to treat a brain disorder in a patient.

Broad aspects of the invention provide any of the following:
a clostridial neurotoxin for use in a method of treating brain tissue damage (e.g. neural tissue damage);
a method of treating brain tissue damage (e.g. neural tissue damage) in a patient, the method comprising administering a clostridial neurotoxin to the patient;
use of a clostridial neurotoxin in the manufacture of a medicament to treat brain tissue damage (e.g. neural tissue damage) in a patient.

Broad aspects of the invention provide any of the following:
a clostridial neurotoxin for use in a method of treating a brain infection;
a method of treating a brain infection in a patient, the method comprising administering a clostridial neurotoxin to the patient;
use of a clostridial neurotoxin in the manufacture of a medicament to treat a brain infection in a patient.

The clostridial neurotoxin may treat the brain disorder by promoting a neuroimmune (e.g. microglial) response. For example, the clostridial neurotoxin may be for use in a method of treating a brain disorder by promoting the neuroimmune (e.g. microglial) response to treat the brain disorder. The clostridial neurotoxin may treat the brain tissue damage (e.g. neural tissue damage) by promoting a neuroimmune (e.g. microglial) response. For example, the clostridial neurotoxin may be for use in a method of treating brain tissue damage (e.g. neural tissue damage) by promoting the neuroimmune (e.g. microglial) response to treat the brain tissue damage (e.g. neural tissue damage).

The clostridial neurotoxin may treat the brain disorder by promoting microglial activity (e.g. in response to neural tissue damage). For example, the clostridial neurotoxin may be for use in a method of treating a brain disorder by promoting microglia activity to treat the brain disorder. The clostridial neurotoxin may treat the brain tissue damage (e.g. neural tissue damage) by promoting a microglial activity (e.g. in response to neural tissue damage). For example, the clostridial neurotoxin may be for use in a method of treating brain tissue damage (e.g. neural tissue damage) by promoting the microglia activity to treat the brain tissue damage (e.g. neural tissue damage).

The clostridial neurotoxin may treat the brain disorder by promoting microglial migration to a site of brain tissue damage (e.g. to neural tissue damage). For example, the clostridial neurotoxin may be for use in a method of treating a brain disorder by promoting microglial migration to a site of brain tissue damage. The clostridial neurotoxin may treat the brain tissue damage (e.g. neural tissue damage) by promoting microglial migration to a site of brain tissue damage (e.g. to neural tissue damage). For example, the clostridial neurotoxin may be for use in a method of treating brain tissue damage (e.g. neural tissue damage) by promoting microglial migration to a site of brain tissue damage (e.g. neural tissue damage) to treat the brain tissue damage.

The clostridial neurotoxin may treat the brain disorder by promoting microglia cell phagocytic activity at a site of brain tissue damage (e.g. neural tissue damage). For example, the clostridial neurotoxin may be for use in a method of treating a brain disorder by promoting microglia cell phagocytic activity at a site of brain tissue damage (e.g. neural tissue damage). The clostridial neurotoxin may treat the brain tissue damage (e.g. neural tissue damage) by promoting microglia cell phagocytic activity at a site of brain tissue damage (e.g. neural tissue damage). For example, the clostridial neurotoxin may be for use in a method of treating brain tissue damage (e.g. neural tissue damage) by promoting microglia cell phagocytic activity at a site of brain tissue damage (e.g. neural tissue damage) to treat the brain tissue damage.

The clostridial neurotoxin may treat the brain disorder by promoting microglial clearance of cellular debris at a site of brain tissue damage (e.g. neural tissue damage). For example, the clostridial neurotoxin may be for use in a method of treating a brain disorder by promoting microglial clearance of cellular debris at a site of brain tissue damage (e.g. neural tissue damage). The clostridial neurotoxin may treat the brain tissue damage (e.g. neural tissue damage) by promoting microglial clearance of cellular debris at a site of brain tissue damage (e.g. neural tissue damage). For example, the clostridial neurotoxin may be for use in a method of treating brain tissue damage (e.g. neural tissue damage) by promoting microglial clearance of cellular debris at a site of brain tissue damage (e.g. neural tissue damage) to treat the brain tissue damage.

The clostridial neurotoxin may treat the brain disorder by promoting a microglia response to an infection. For example, the clostridial neurotoxin may be for use in a method of treating a brain disorder by promoting the microglia response to an infection to treat the brain disorder. The clostridial neurotoxin may treat the brain tissue damage (e.g. neural tissue damage) by promoting a microglia response to an infection. For example, the clostridial neurotoxin may be for use in a method of treating brain tissue damage (e.g. neural tissue damage) by promoting a microglia response to an infection to treat the brain tissue damage (e.g. neural tissue damage).

The clostridial neurotoxin may treat a brain infection by promoting a neuroimmune (e.g. microglial) response. For example, the clostridial neurotoxin may be for use in a method of treating a brain infection by promoting the neuroimmune (e.g. microglial) response to treat the brain infection. The clostridial neurotoxin may treat the brain infection by promoting a neuroimmune (e.g. microglial) response. For example, the clostridial neurotoxin may be for use in a method of treating a brain infection by promoting the neuroimmune (e.g. microglial) response to treat the brain infection.

The clostridial neurotoxin may treat a brain infection by promoting microglia cell activity (e.g. in response to infection). For example, the clostridial neurotoxin may be for use in a method of treating a brain infection by promoting microglia cell activity to treat the brain infection. The clostridial neurotoxin may treat the brain infection by promoting microglia cell activity (e.g. in response to infection). For example, the clostridial neurotoxin may be for use in a method of treating a brain infection by promoting microglia cell activity to treat the brain infection.

The clostridial neurotoxin may treat a brain infection by promoting microglia cell migration to a site of a brain infection. For example, the clostridial neurotoxin may be for use in a method of treating a brain infection by promoting microglia cell migration to a site of a brain infection. The clostridial neurotoxin may treat a brain infection by promoting microglial migration to a site of a brain infection. For example, the clostridial neurotoxin may be for use in a method of treating a brain infection by promoting microglia cell migration to a site of brain infection to treat the brain infection.

The clostridial neurotoxin may treat a brain infection by promoting microglia cell phagocytic activity at a site of brain infection. For example, the clostridial neurotoxin may be for use in a method of treating a brain infection by promoting microglia cell phagocytic activity at a site of brain infection. The clostridial neurotoxin may treat a brain infection by promoting microglia cell phagocytic activity at a site of brain infection. For example, the clostridial neurotoxin may be for use in a method of treating a brain infection by promoting microglia cell phagocytic activity at a site of brain infection to treat the brain infection.

The clostridial neurotoxin may treat a brain infection by promoting microglial clearance of cellular debris at a site of brain infection. For example, the clostridial neurotoxin may be for use in a method of treating a brain infection by promoting microglial clearance of cellular debris at a site of brain infection. The clostridial neurotoxin may treat the brain infection by promoting microglial clearance of cellular debris at a site of brain infection. For example, the clostridial neurotoxin may be for use in a method of treating a brain infection by promoting microglial clearance of cellular debris at a site of brain infection to treat the brain infection.

The clostridial neurotoxin may treat a brain infection by promoting a microglia response to an infection. For example, the clostridial neurotoxin may be for use in a method of treating a brain infection by promoting the microglia response to an infection to treat the brain disorder. The clostridial neurotoxin may treat the brain infection by promoting a microglia response to an infection. For example, the clostridial neurotoxin may be for use in a method of treating a brain infection by promoting a microglia response to an infection to treat the brain infection.

In one aspect there is provided a clostridial neurotoxin for use in a method of treating brain disorder in a patient by promoting a neuroimmune response. In other words, one aspect provides a clostridial neurotoxin for use in promoting a neuroimmune response to treat a brain disorder.

In a related aspect there is provided a method of treating a brain disorder in a patient by promoting a neuroimmune response, the method comprising administering a clostridial neurotoxin to the patient. In other words, one aspect provides a method for promoting a neuroimmune response to treat a brain disorder in a patient, the method comprising administering a clostridial neurotoxin to the patient.

In a further aspect there is provided use of a clostridial neurotoxin in the manufacture of a medicament to treat a brain disorder in a patient, by promoting a neuroimmune response. In other words, one aspect provides use of a clostridial neurotoxin in the manufacture of a medicament for promoting a neuroimmune response to treat a brain disorder in a patient.

Thus, the clostridial neurotoxin described herein promotes a neuroimmune response. As such, clostridial neurotoxin(s) find utility in treating a brain disorder. The term “brain disorder” (which may be used herein synonymously with the term “brain damage”) as used herein is a disorder that can be treated by promoting a neuroimmune response in a patient. The brain disorder may be caused by brain tissue damage (e.g. neural tissue damage). Additionally or alternatively, the brain disorder may cause neural tissue damage. As such, the promotion of neuroimmune response activity may have the effect of promoting repair of neural tissue damage, thus enhancing recovery from the brain disorder.

The term “brain tissue damage” may be used synonymously herein with the term “brain tissue injury”. Similarly, the term “neural tissue damage” may be used synonymously herein with the term “neural tissue injury”.

In one embodiment, the neuroimmune response may be defined as a microglial response. Thus, the clostridial neurotoxin promotes microglial activity in the brain.

Microglia represent a population of macrophage-like cells in the central nervous system (particularly the brain). They are typically considered immune sentinels that are capable of orchestrating a potent inflammatory response. Microglia are also involved in synaptic organization, trophic neuronal support during development, phagocytosis of apoptotic cells in the developing brain, myelin turnover, control of neuronal excitability, phagocytic debris removal as well as brain protection and repair.

In one aspect there is provided a clostridial neurotoxin for use in a method of treating a brain disorder in a patient by promoting microglia activity (e.g. in the brain). In other words, one aspect provides a clostridial neurotoxin for use in promoting microglia activity (e.g. in the brain) to treat a brain disorder. In the context of brain tissue (e.g. neural tissue) damage, microglia activity may be promoted to repair (or clear) damaged tissue. A non-limiting example of microglial cell (e.g. macrophages) activity includes phagocytic activity (e.g. clearance of cellular debris) at a site of the nerve or brain tissue damage.

Thus, without wishing to be bound by theory, one observed effect on which the present invention is predicated is the ability to promote the neuroimmune response's role in responding to neural damage (which may be caused by, or indeed causing, the brain disorder), to clear/repair such damage. Thus, while an individual's endogenous neuroimmune response may already come into action and work to combat neural damage as a matter of course, the present invention provides an advantageous ‘boost’ to such response, which may correlate with faster onset of the neuroimmune response, increased recruitment of neuroimmune cells to a site of neural damage and/or increased activity of neuroimmune cells toward treating the damage. As an outcome, enhanced recovery from injury (e.g. neural tissue injury) may follow.

To demonstrate such technical effects, the inventors coupled “two-photon” imaging and laser technology with a transgenic mouse model allowing for high-resolution tracking of the activity of the animal's own microglia cells, directly within the in vivo brain setting. The animal model (CX3CR1-GFP mice) express GFP within their microglia cells, allowing the cells to imaging and tracked via fluorescent light microscopy.

In order to induce neural damage representative of that which occurs in a ‘brain disorder’ (e.g. in a brain infection) described herein, the inventor employed two-photon microscopy technology to provide a distinct focal point of laser-induced injury in the brain. In more detail, a micrometre-size region of interest (ROI) was selected in the focal plane at the depth of 25-micrometres from the cortical surface and the laser intensity was applied to this focal region to provide a localised, distinct region of damaged brain tissue. Such distinct region of brain tissue damage emulates that which may occur due to brain hypoxia (e.g. following a stroke), due to a growing brain tumor and/or due to swelling (e.g. encephalitis) following infection, while also allowing investigations of microglia cells around a distinct, space-controlled area of induce tissue damage.

As demonstrated in the examples section, the microglia responded to this damage via migration to the particular site of damage, and adopting a phenotype consistent with phagocytic activity. However, surprisingly, such activity was significantly increased in mice to which a clostridial neurotoxin (e.g. BoNT/A) was administered, demonstrating an unexpected technical effect for clostridial neurotoxins in promoting a patient's neuroimmune response to brain damage.

Thus, the clostridial neurotoxins find utility in enhancing recovery from brain (e.g. neural tissue) damage, and thus can be used to treat a distinct clinical subset of patient requiring brain damage repair. Advantageously, such repair is orchestrated by the patient's own neuroimmune response, which is enhanced/promoted following administration of a clostridial neurotoxin. The step(s) toward providing this effect, namely administration of the clostridial neurotoxin through routes such as intracortical injection, are comparatively (vs brain surgery) achievable and non-invasive techniques. Thus, need for complex surgical intervention (brain surgery) can be mitigated.

Following on from the note that patients suffering brain damage are particularly suited to treatment by the present invention, a yet further clinical subset (or sub-subset) of such patients may be represented by those patients in which the ‘endogenous’ level of neuroimmune response activity is insufficient to treat the tissue damage. In other words, the presently demonstrated technical effect(s) provided by administration of a clostridial neurotoxin provide particular clinical utility in patients that would benefit from a ‘boost’ in their neuroimmune response (to injury) in order to improve their prognosis vs that which would follow when relying on the patients' endogenous level of neuroimmune response.

Such utility contrasts with the purported function of clostridial neurotoxins in prior art methods employing such neurotoxins for treating brain disorders. Since clostridial neurotoxins function to cleave SNARE proteins and suppress neurotransmitter secretion (as explained in detail elsewhere herein), prior art methods have sought to suppress neuronal activity in the brain via administration of clostridial neurotoxin, in order to effect neurotoxin-mediated prevention of neurotransmitter release. For example, it has been proposed to target neurotoxins to regions of the brain showing ‘abnormal’ activity in epilepsy, in order to suppress such abnormal activity which may otherwise correlate with seizure. Other prior art has proposed to target (and suppress) otherwise hyperactive inhibitory cholinergic neurons (e.g. present in the striatum of brain) to suppress neural activity correlating with movement disorders.

However, such prior art methods did not involve repair of tissue damage, let alone via clostridial neurotoxin-induced promotion of neuroimmune cell activity. Instead of suppressing neuronal activity, the present invention is directed to removal/repair of damaged neurons. Thus, the invention is directed to treating brain disorders via treating brain tissue damage; more particularly, via promoting neuroimmune cell activity in treating such damage.

Thus, not only has the present inventor identified a new technical field of application (i.e. brain disorders associated with brain tissue damage) for clostridial neurotoxins, but also identified an unexpected technical effect within said field (i.e. promotion of the neuroimmune response). In a related aspect there is provided a method of treating a brain disorder in a patient by promoting microglia activity (e.g. in the brain), the method comprising administering a clostridial neurotoxin to the patient. In other words, one aspect provides a method for promoting microglia activity (e.g. in the brain) to treat a brain disorder in a patient, the method comprising administering a clostridial neurotoxin to the patient.

In a further aspect there is provided use of a clostridial neurotoxin in the manufacture of a medicament to treat a brain disorder in a patient, by promoting microglia activity (e.g. in the brain). In other words, one aspect provides use of a clostridial neurotoxin in the manufacture of a medicament for promoting microglia activity (e.g. in the brain) to treat a brain disorder in a patient.

Thus, the clostridial neurotoxin described herein promotes a neuroimmune response. As such, clostridial neurotoxin(s) find utility in treating a brain disorder. The term “brain disorder” (which may be used herein synonymously with the term “brain damage”) as used herein is a disorder that can be treated by promoting a neuroimmune response in a patient.

The term “neuroimmune response” refers to the response of a patient's neuroimmune system to a brain disorder. For example, the term “neuroimmune response” as used herein may refer to the response of the neuroimmune system to brain tissue damage (e.g. neural tissue damage) in the brain, infection in the brain and/or a brain tumor (said neural damage may be caused by infection in the brain and/or a brain tumor). The neuroimmune response may be to prevent brain tissue damage (e.g. neural tissue damage). Additionally or alternatively, the neuroimmune response may be to repair brain tissue damage (e.g. neural tissue damage). The term “neuroimmune response” is not intended to be limited to a response to infection, and additionally embraces response of the neuroimmune system to brain tissue damage (e.g. neural tissue damage) cause by alternative disorders (e.g. stroke). Indeed, physical injury and necrosis can also cause a neuroimmune response. In embodiments where the brain disorder is (or is caused by) and infectious disease, the neuroimmune response may be a response to infection.

The neuroimmune system is composed primarily of glial cells, including astrocytes, microglia, and oligodendrocytes, with microglia cells (e.g. macrophages) in particular representing key immune cells of this system in the brain. As described above, the neuroimmune response as referred to herein may be defined as a microglial response.

The term “promotes a neuroimmune response” may mean that the clostridial neurotoxin (or fragment thereof) of the invention initiates a neuroimmune response in the patient having a brain disorder, for example where a neuroimmune response was not occurring or where the ‘endogenous’ neuroimmune response was not sufficiently strong to overcome the brain disorder (e.g. neural tissue damage). Thus, while the invention is predicated on administration of a clostridial neurotoxin in the methods described herein, it may be more accurate to say that the “neuroimmune response” (preferably microglial cells) effect the treatment e.g. of brain tissue damage; in this context, the clostridial neurotoxin promotes the role of the neuroimmune response (e.g. microglia cells) in treating brain tissue damage.

The term “promotes a neuroimmune response” may mean that the clostridial neurotoxin of the invention accelerates the onset of a neuroimmune response in the patient having a brain disorder, for example where a neuroimmune response was not occurring. In preferable embodiments, the term “promotes a neuroimmune response” may mean that the clostridial neurotoxin increases the level of the neuroimmune response. Said increase may be an increase when compared to the level of the neuroimmune response in the absence of the clostridial neurotoxin of the invention. In one embodiment, the neuroimmune response may remove damaged brain tissue (e.g. damaged neural tissue or a nerve) allowing for the rebuilding of damaged neuronal circuits, thereby restoring activity and/or neuronal communication in a network or population of neurons.

In an embodiment, the neuroimmune response comprises activation of microglial cell(s) (e.g. macrophages), as measured by 1) changes in the density of microglial cells at a site of brain tissue damage (e.g. neural tissue damage), 2) changes in the level of microglia cell migration (e.g. changes in sphericity of microglia cells) to a site of brain tissue damage (e.g. neural tissue damage), 3) changes in the level of microglia process extension toward a site of brain tissue damage (e.g. neural tissue damage), and/or 4) changes in the level of microglia cell phagocytic activity (e.g. clearance of cellular debris) at a site of brain tissue damage (e.g. neural tissue damage) in the presence of a clostridial neurotoxin of the invention when compared to microglia activity in the absence of the clostridial neurotoxin of the invention.

It should be noted that neuroimmune cell (notably microglia cell) activity is expected to increase also in an ‘untreated’ (e.g. untreated with an agent such as a clostridial neurotoxin) brain in response to neural tissue damage, consistent with such cells' role in central nervous system repair after injury. Notwithstanding this, the ‘control’ experiments provided by the present application (e.g. vehicle-only control) demonstrate that administration of a clostridial neurotoxin provides for levels of injury-induced activation of neuroimmune cell activity that goes above-and-beyond a level otherwise endogenous to the subject (even in the presence of precisely the same type of injury). Thus, in promoting neuroimmune cell activity (responsive to damage), the present invention can be utilised to ‘boost’ such cell's efforts to combat/repair damage caused by a brain disorder, further enhancing patient healing and mitigating further insults caused by the presence of damaged tissue (e.g. necrotic material). For example, methods of the invention may be utilised to treat patients whose ‘normal’ (or ‘endogenous’) level of neuroimmune cell activity (responsive to damage) is insufficient to combat/repair neural tissue injury underlying (or caused by) the brain disorder.

By ‘promoting’ the neuroimmune response to increase its activity by a certain level, there may a concomitant increase in the level of ‘damage repair’. As a positive outcome, a subsequent (e.g. correlating) increase in the patient's prognosis for recovery (or survival) may follow. For example, by ‘promoting’ the neuroimmune response to increase its activity by say 5%, there may a concomitant 5% increase the level of ‘damage repair’; and a subsequent 5% increase in the patient's prognosis for recovery (or survival) may follow.

For instance, the neuroimmune response may be increased by at least 1%, 2%, 3%, 4%, 5%, 6%, 7%, 8%, 9% or 10% (preferably at least 5%) or more in the presence of a clostridial neurotoxin of the invention when compared to the neuroimmune response in the absence of the clostridial neurotoxin of the invention (or in the presence of an alternative polypeptide).

The neuroimmune response may be increased by at least 5%, 10%, 20%, 30%, 40%, 50%, 60% or 70% (preferably at least 30%) in the presence of a clostridial neurotoxin of the invention when compared to the neuroimmune response in the absence of the clostridial neurotoxin of the invention (or in the presence of an alternative polypeptide). In some embodiments the neuroimmune response may be increased by at least 100%, 150% or 200% in the presence of a clostridial neurotoxin of the invention when compared to the neuroimmune response in the absence of the clostridial neurotoxin of the invention (or in the presence of an alternative polypeptide).

The neuroimmune response may refer to microglia activity. For example, the neuroimmune response referred to herein may preferably comprise or consist of microglia activity. In one embodiment, the clostridial neurotoxin promotes microglia activity in the brain. For instance, the microglia activity may be increased by at least 1%, 2%, 3%, 4%, 5%, 6%, 7%, 8%, 9% or 10% (preferably at least 5%) or more in the presence of a clostridial neurotoxin of the invention when compared to microglia activity in the absence of the clostridial neurotoxin of the invention (or in the presence of an alternative polypeptide).

For example, microglia activity may be increased by at least 5%, 10%, 20%, 30%, 40%, 50%, 60% or 70% (preferably at least 30%) in the presence of a clostridial neurotoxin of the invention when compared to microglia activity in the absence of the clostridial neurotoxin of the invention (or in the presence of an alternative polypeptide). In some embodiments, microglial activity may be increased by at least 100%, 150% or 200% in the presence of a clostridial neurotoxin of the invention when compared to microglial activity in the absence of the clostridial neurotoxin of the invention (or in the presence of an alternative polypeptide).

The term “microglia activity” (aka “microglia cell activity” or “microglial activity”) as used refers to functions undertaken by microglia cells against the presence of an insult such as brain tissue damage, infection, and/or a tumour. Microglia are sensitive to neural tissue damage, and such damage by an injury/infection/neurotoxicant etc. stimulates microglia (e.g. to become activated) which migrate to the site of damage. Stronger microglia responses typically correlate with higher numbers of microglial cells migrating to a lesion (site of damage), such that the density of microglial cells at a site of injury may increase in line with the rate/level of the response.

In one embodiment, microglia activity may be measured based on the density of microglial cells at a site of brain tissue damage (e.g. neural tissue damage). The clostridial neurotoxin may promote “microglia cell density level” at a site of brain tissue damage (e.g. neural tissue damage), for example brain tissue damage caused by a brain disorder described herein.

The term “microglia cell density level” refers to the number of microglia cells at a given site/in a given area, with increasing density correlating with increasing numbers of cells.

The clostridial neurotoxin may promote a microglia cell density level at a site of infection (e.g. a site of brain tissue damage caused by infection). The clostridial neurotoxin may promote a microglia cell density level at a site of a brain tumor (or a site of brain tissue damage caused by a brain tumor). The clostridial neurotoxin may promote a microglia cell density level at a site of a stroke (or a site of brain tissue damage caused by a stroke). For instance, the microglia cell density level may be increased by at least 1%, 2%, 3%, 4%, 5%, 6%, 7%, 8%, 9% or 10% (preferably at least 5%) or more in the presence of a clostridial neurotoxin of the invention when compared to the microglia cell density level in the absence of the clostridial neurotoxin of the invention (or in the presence of an alternative polypeptide).

For example, said microglia cell density level (in any embodiment described herein) may be increased by at least 5%, 10%, 20%, 30%, 40%, 50%, 60% or 70% (preferably at least 30%) in the presence of a clostridial neurotoxin of the invention when compared to microglia cell density level in the absence of the clostridial neurotoxin of the invention (or in the presence of an alternative polypeptide). In some embodiments, said microglia cell density level may be increased by at least 100%, 150% or 200% in the presence of a clostridial neurotoxin of the invention when compared to microglia cell density in the absence of the clostridial neurotoxin of the invention (or in the presence of an alternative polypeptide).

By increasing microglia cell density level, as such, at a site of damage (neural injury), the number of microglia cells that are ‘available’ to combat/repair the injury is increased. For example, due to an increased number of microglia being present at the site of damage/injury, the amount of microglial phagocytosis of damaged tissue (and/or pathogen) can follow. A concomitant increase in the level of repair (of the injury) can therefore follow, following which treatment of the brain disorder is provided.

In one embodiment, microglia activity may be measured based on a level of “microglia cell migration” to a site of brain tissue damage (e.g. neural tissue damage). In one embodiment, the clostridial neurotoxin promotes microglia cell migration to a site of brain tissue damage (e.g. neural tissue damage). The clostridial neurotoxin may promote microglia cell migration to a site of infection (or a site of brain tissue damage caused by infection). The clostridial neurotoxin may promote microglia cell migration to a site of a brain tumor (or a site of brain tissue damage caused by a brain tumor). The clostridial neurotoxin may promote a microglia cell density level at a site of a stroke (or a site of brain tissue damage caused by a stroke).

The term “microglia cell migration” refers to the active (directional) movement microglia cells moving toward a site described herein (e.g. site of brain tissue damage, infection, tumour), for example responsive to a stimulant release from dying cells. The term “microglia cell migration” may be used synonymously with the term “microglial migration” herein.

For instance, said microglia cell migration may be increased by at least 1%, 2%, 3%, 4%, 5%, 6%, 7%, 8%, 9% or 10% (preferably at least 5%) in the presence of a clostridial neurotoxin of the invention when compared to the microglia cell density level in the absence of the clostridial neurotoxin of the invention (or in the presence of an alternative polypeptide). For example, said microglia cell migration (in any embodiment described herein) may be increased by at least 5%, 10%, 20%, 30%, 40%, 50%, 60% or 70% (preferably at least 30%) in the presence of a clostridial neurotoxin of the invention when compared to microglia cell migration in the absence of the clostridial neurotoxin of the invention (or in the presence of an alternative polypeptide). In some embodiments, said microglia cell migration may be increased by at least 80%, 90% or 100% in the presence of a clostridial neurotoxin of the invention when compared to microglia cell migration in the absence of the clostridial neurotoxin of the invention (or in the presence of an alternative polypeptide). In some embodiments, said microglia cell migration may be increased by at least 110%, 125% or 150% in the presence of a clostridial neurotoxin of the invention when compared to microglia cell migration in the absence of the clostridial neurotoxin of the invention (or in the presence of an alternative polypeptide).

By increasing microglia cell migration toward a site described herein (e.g. site of brain tissue damage, infection, tumour), the number of microglia cells that are ‘available’ to combat/repair the injury (or infection) is increased. For example, due to an increased number of microglia being present at the site of damage/injury, the amount of microglial phagocytosis of damaged tissue (and/or pathogen) can follow. A concomitant increase in the level of repair (of the injury) can therefore follow, following which treatment of the brain disorder is provided.

A suitable readout of cell migration is the sphericity of microglia cells. Thus, a sphericity index for a microglial cell (more typically a mean sphericity index for microglial cells) may be detected, where a decrease in sphericity index correlates with an increased elongation (elongated shape) of the microglial cell(s), e.g. because they have become motile and are migrating in the direction of the site of brain damage. The sphericity index can be defined as the ratio of the squared volume (V) of a microglial cell over the cube of the surface area (S) covered by the microglial cell:

$$sphericity = 36 π \frac{{V}^{2}}{{S}^{3}}$$

For example, the sphericity of microglial cell(s) may be decreased by at least 1%, 2%, 5%, 10%, 15%, 20%, 25% or 30% (preferably at least 10%) in the presence of a clostridial neurotoxin of the invention when compared to sphericity of microglial cell(s) in the absence of the clostridial neurotoxin of the invention (or in the presence of an alternative polypeptide). In some embodiments, sphericity of microglial cell(s) may be decreased by at least 35%, 40% or 50% in the presence of a clostridial neurotoxin of the invention when compared to sphericity of microglial cell(s) in the absence of the clostridial neurotoxin of the invention (or in the presence of an alternative polypeptide).

Microglia are highly dynamic cells that interact with neurons and nonneuronal cells. Microglia patrol the brain parenchyma via continuous process extension and retraction. For instance, microglia can extend their processes toward damaged areas, for example in response to stimulation of the metabotropic ATP receptor P2Y(12) by ATP released from damaged tissue.

In one embodiment, microglia activity may be measured based on a level of microglia process extension toward a site of brain tissue damage (e.g. neural tissue damage). In one embodiment, the clostridial neurotoxin promotes microglia process extension toward a site of brain tissue damage (e.g. neural tissue damage). The clostridial neurotoxin may promote microglia process extension toward a site of infection (or a site of brain tissue damage cause by infection). The clostridial neurotoxin may promote microglia process extension toward a site of a brain tumor (or a site of brain tissue damage caused by a brain tumor). The clostridial neurotoxin may promote microglia process extension toward a site of a stroke (or a site of brain tissue damage caused by a stroke).

The term “microglia process extension” refers to an extension in length of the microglia cell's elongated processes, said processes enabling, amongst other functions, the cells to migrate such as via chemotaxis. For example, the number and/or length of microglial processes may be increased in methods of the invention.

For instance, said microglia process extension may be increased by at least 1%, 2%, 3%, 4%, 5%, 6%, 7%, 8%, 9% or 10% (preferably at least 5%) in the presence of a clostridial neurotoxin of the invention when compared to the microglia cell density level in the absence of the clostridial neurotoxin of the invention (or in the presence of an alternative polypeptide). For example, said microglia process extension (in any embodiment described herein) may be increased by at least 5%, 10%, 20%, 30%, 40%, 50%, 60% or 70% (preferably at least 60%) in the presence of a clostridial neurotoxin of the invention when compared to microglia process extension in the absence of the clostridial neurotoxin of the invention (or in the presence of an alternative polypeptide). In some embodiments, said microglia process extension may be increased by at least 80%, 90% or 100% in the presence of a clostridial neurotoxin of the invention when compared to microglia process extension in the absence of the clostridial neurotoxin of the invention (or in the presence of an alternative polypeptide). In some embodiments, said microglia process extension may be increased by at least 125%, 150% or 200% in the presence of a clostridial neurotoxin of the invention when compared to microglia process extension in the absence of the clostridial neurotoxin of the invention (or in the presence of an alternative polypeptide).

In one embodiment, microglia activity may be measured based on a level of microglia cell phagocytic activity at a site of brain tissue damage (e.g. neural tissue damage). The clostridial neurotoxin may promote microglia cell phagocytic activity at a site of brain tissue damage (e.g. neural tissue damage). The clostridial neurotoxin may promote microglia cell phagocytic activity at a site of infection (or. a site of brain tissue damage caused by infection). The clostridial neurotoxin may promote microglia cell phagocytic activity at a site of a brain tumor (or a site of brain tissue damage caused by a brain tumor). The clostridial neurotoxin may promote microglia cell phagocytic activity a site of a stroke (or a site of brain tissue damage caused by a stroke).

The term “microglia cell phagocytic activity” (aka “microglial phagocytic activity”) refers to engulfment of cellular material by microglia (embracing engulfment of cell debris following brain tissue damage, as well as foreign bodies such as microorganism). When engulfing cell debris, the term “microglia cell phagocytic activity” may be used synonymously with the term “microglial clearance of cellular debris” herein. Phagocytic activity may be measured by detecting clearance of cell debris at a site of brain tissue damage.

The term “cell debris” preferably refers to cellular components released from a dead neuron.

For example, said microglia cell phagocytic activity (in any embodiment described herein) may be increased by at least 10%, 20%, 30%, 40%, 50%, 60% or 70% (preferably at least 30%) in the presence of a clostridial neurotoxin of the invention when compared to microglia cell phagocytic activity in the absence of the clostridial neurotoxin of the invention (or in the presence of an alternative polypeptide). In some embodiments, said microglia cell phagocytic activity may be increased by at least 80%, 90% or 100% in the presence of a clostridial neurotoxin of the invention when compared to microglia cell phagocytic activity in the absence of the clostridial neurotoxin of the invention (or in the presence of an alternative polypeptide). In some embodiments, said microglia cell phagocytic activity may be increased by at least 125%, 150% or 200% in the presence of a clostridial neurotoxin of the invention when compared to microglia cell phagocytic activity in the absence of the clostridial neurotoxin of the invention (or in the presence of an alternative polypeptide).

The inventors have surprisingly demonstrated that the clostridial neurotoxin-mediated promotion of a neuroimmune response can occur rapidly i.e. can promote early onset of the response to damage. Furthermore, the promoted response may be acute e.g. abating once the tissue damage/invading insult has been cleared, mitigating any negative effect of a long-term ‘hyperactive’ response.

In one embodiment, the neuroimmune response is promoted ≤2 minutes, ≤4 minutes, ≤6 minutes, ≤8 minutes, ≤10 minutes, ≤12 minutes, ≤14 minutes, ≤16 minutes, ≤18 minutes or ≤20 minutes following the occurrence of brain tissue damage (e.g. neuron tissue damage). In a preferable embodiment, the neuroimmune response is promoted ≤10 minutes following the occurrence of brain tissue damage.

The term “brain tissue damage” (e.g. neuron tissue damage) may be damage that is caused by a brain disorder described herein. For example, where the brain disorder is an infection, the brain tissue damage may be caused by a microbial infection.

In one embodiment, the neuroimmune response is promoted ≤2 minutes, ≤4 minutes, ≤6 minutes, ≤8 minutes, ≤10 minutes, ≤12 minutes, ≤14 minutes, ≤16 minutes, ≤18 minutes or ≤20 minutes following administration of the clostridial neurotoxin. In a preferable embodiment, the neuroimmune response is promoted ≤10 minutes following administration of the clostridial neurotoxin.

The neuroimmune response may be promoted for ≤48 hours, ≤36 hours, ≤24 hours, or ≤12 hours (preferably ≤24 hours) following the occurrence of brain tissue damage. For example, there may be no clostridial neurotoxin-mediated promotion of the neuroimmune after said time period; in other words, there may be no clostridial neurotoxin-mediated promotion of the neuroimmune response after 48 hours, ≤36 hours, ≤24 hours, or ≤48 hours (preferably ≤24 hours) following the occurrence of brain tissue damage. In such embodiments, the clostridial neurotoxin may be said to promote an acute neuroimmune response. The term “no clostridial neurotoxin-mediated promotion” means that there is substantially no increase in the neuroimmune response, for example “substantially no increase” suitably meaning the increase in the neuroimmune response may be less than 5%, 3%, or 1% (preferably ≤0%, where there is no increase in the neuroimmune response at all).

As described above, a brain disorder/brain damage herein is a disorder that can be treated by promoting a neuroimmune response in a patient.

Such disorders may originate from within or outside the brain, and include disorders associated with bodily insults that cause brain tissue damage. By way of example, in stroke, a blockage of the blood vessels may interrupt or reduce the supply of blood to the brain, leading to oxygen depletion in the brain and thus nerve cell death. The nerve damage thus caused may underly symptoms suffered by a patient that has said brain disorder.

Thus, a clostridial neurotoxin of the invention may be for treating a brain disorder that causes brain tissue damage (e.g. neural tissue damage), e.g. by promoting a neuroimmune response.

Examples of brain disorders embraced by the invention include any one (or more) of traumatic brain injury, cancer (e.g. a brain tumour), infectious disease, stroke, a neurodegenerative disorder, brain aneurysm, multiple sclerosis, anoxic injury, toxic injury and metabolic injury.

The “brain disorder” may refer more specifically to brain tissue damage (e.g. neural tissue damage). In such cases, it may be said that the brain disorder is caused by any one (or more) of traumatic brain injury, cancer (e.g. a brain tumour), infectious disease, stroke, a neurodegenerative disorder, brain aneurysm, multiple sclerosis, anoxic injury, toxic injury and metabolic injury.

In a preferable embodiment, the brain disorder is caused by a non-traumatic brain injury. A non-traumatic brain injury can be the result of an illness, oxygen deprivation, metabolic disorders, aneurysms, and/or cardiac arrest. A non-traumatic brain injury may be distinguished from a “traumatic brain injury” (TBI), which is typically caused by as physical blow or jolt to the head or a penetrating head injury that disrupts the function of the brain.

In one embodiment, the brain disorder is not caused by a “traumatic brain injury” (TBI).

A non-traumatic brain injury may be defined by one or more selected from: cancer (e.g. a brain tumour), infectious disease, stroke, a neurodegenerative disorder, brain aneurysm, multiple sclerosis, anoxic injury, toxic injury and metabolic injury (preferably cancer, infectious disease, and stroke; more preferably infectious disease).

Preferable examples of a brain disorder of the invention may be selected from brain disorder by cancer, infectious disease, and stroke. In other words, the brain disorder may be caused by cancer, infectious disease, and/or stroke.

A particularly suitable brain disorder is infectious disease. In other words, the brain disorder may be caused by infectious disease.

Infections can cause inflammation of the brain (encephalitis). Viruses are the most common causes of encephalitis. Infections can also cause inflammation of the layers of tissue (meninges) that cover the brain, the resulting condition being called meningitis. Often, bacterial meningitis spreads to the brain itself, causing encephalitis. Similarly, viral infections that cause encephalitis often also cause meningitis. When both the brain and the meninges are infected, the disorder is called meningoencephalitis. However, infection that affects mainly the meninges is usually called meningitis, and infection that affects mainly the brain is usually called encephalitis.

The infectious disease may refer to an infection with a microorganism, for example a microorganism selected from a bacterium, virus, fungus, a parasite and a protist (more preferably selected from a bacterium and a fungus). Thus, the infectious disease may be caused by an infectious agent which contains nucleic acids (DNA, RNA or both), which may contrast an agent of a prion protein infection. The infectious disease may thus be distinguished from a prion disease.

In one embodiment, the infectious disease is selected from encephalitis, meningitis, and a brain abscess (preferably encephalitis, and meningitis). Thus, in one embodiment, the brain disorder may be encephalitis. In one embodiment, the brain disorder may be meningitis.

In one embodiment, the brain disorder is caused by a neurodegenerative disease. Said neurodegenerative disease may be selected from: Alzheimer's disease, Parkinson's disease, Parkinson's disease related disorders, motor neuron disease (e.g. amyotrophic lateral sclerosis), prion disease, Huntington's disease, spinocerebellar ataxia, ataxia, Hallervorden-Spatz disease, and frontotemporal lobar degeneration.

In one embodiment, the brain disorder is caused by stroke. Said stroke may be ischemic stroke (e.g. lack of blood flow to brain) or hemorrhagic stroke (e.g. bleeding within the brain).

A “patient” (which may be used synonymously with the term “subject”) as used herein may be a mammal, such as a human or other mammal. Preferably “patient” means a human subject.

The patient may be a patient having a lower than average (basal) neuroimmune response, e.g. a patient receiving an alternative therapy, such as chemotherapy, which may suppress the immune system. For example, the patient may be immunocompromised. The patient may be immunocompromised due to any underlying condition, or due to an elderly state (e.g. patients >60 or 70 years old). Additionally or alternatively, the patient may be immunocompromised due to administration of an alternatives therapy (for example, as mentioned above, chemotherapy). The latter represents a distinct subset of patients in which the state of being immunocompromised is ‘induced’, and such patients may benefit in particular from the treatment described herein.

The term “disorder” as used herein also encompasses a “disease”. In one embodiment the disorder is a disease.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment.

The term “treat” or “treating” as used herein refers to the disorder and/or a symptom thereof.

Therefore, a clostridial neurotoxin of the invention may be administered to a patient in a therapeutically effective amount or a prophylactically effective amount. Preferably a clostridial neurotoxin of the invention is administered to a patient in a therapeutically effective amount.

A “therapeutically effective amount” is any amount of the clostridial neurotoxin, which when administered alone or in combination, with another agent, to a patient for treating said disorder (or a symptom thereof) is sufficient to effect such treatment of the disorder, or symptom thereof.

A “prophylactically effective amount” is any amount of the clostridial neurotoxin that, when administered alone or in combination to a patient inhibits or delays the onset or reoccurrence of a disorder (or a symptom thereof). In some embodiments, the prophylactically effective amount prevents the onset or reoccurrence of a disorder entirely. “Inhibiting” the onset means either lessening the likelihood of a disorder's onset (or symptom thereof), or preventing the onset entirely.

The clostridial neurotoxins of the invention may be formulated in any suitable manner for administration to a subject, for example as part of a pharmaceutical composition. Thus, in one aspect, the invention provides a pharmaceutical composition for any method of treatment described herein, the pharmaceutical composition comprising a clostridial neurotoxin of the invention and a pharmaceutically acceptable carrier, excipient, adjuvant, propellant and/or salt.

In some embodiments, the clostridial neurotoxin of the invention may be in a single-chain form, while in other embodiments the clostridial neurotoxin may be in a di-chain form, e.g. where the two chains are linked by a di-sulphide bridge. Preferably the clostridial neurotoxin is in a di-chain form.

The clostridial neurotoxins of the present invention may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In the case of a clostridial neurotoxin that is to be delivered locally, the clostridial neurotoxin may be formulated as a cream (e.g. for topical application), or for sub-dermal injection.

Local delivery means may include an aerosol, or other spray (e.g. a nebuliser). In this regard, an aerosol formulation of a clostridial neurotoxin enables delivery to the lungs and/or other nasal and/or bronchial or airway passages.

Clostridial neurotoxins of the invention may be administered to a subject by intrathecal or epidural injection in the spinal column at the level of the spinal segment involved in the innervation of an affected organ.

A route of administration may be via or localised injection. In one embodiment a clostridial neurotoxin of the invention is administered at or near to a site of injury, preferably at a site of injury. In one embodiment the route of administration of a clostridial neurotoxin of the invention may be perineural, intraneural, intraspinal, and/or intrathecal.

The clostridial neurotoxin may be administered subcutaneously and/or intracranially. For example, the clostridial neurotoxin may be administered intracranially (e.g. to any appropriate region of the brain in which neural damage is present). For instance, the clostridial neurotoxin may be administered by intracortical administration (e.g. intracortical injection). The clostridial neurotoxin may be administered into the somatosensory cortex. The clostridial neurotoxin may be administered into the frontal cortex.

Intracranial administration (such as intracranial injection) may be used to allow for direct administration to the brain.

The dosage ranges for administration of the clostridial neurotoxins of the present invention are those to produce the desired therapeutic and/or prophylactic effect. It will be appreciated that the dosage range required depends on the precise nature of the clostridial neurotoxin or composition, the route of administration, the nature of the formulation, the age of the subject, the nature, extent or severity of the subject's condition, contraindications, if any, and the judgement of the attending physician. Variations in these dosage levels can be adjusted using standard empirical routines for optimisation.

In one embodiment a dosage of the clostridial neurotoxin is a flat dose. A flat dose may be in the range of 50 μg to 250 μg, preferably ≤100 μg to 100 μg. In one embodiment a flat dose may be at least 50 μg, 100 μg, 500 μg, 1 ng, 50 ng, 100 ng, 500 ng, 1 μg or 50 μg. Said dose may be a single flat dose.

Fluid dosage forms are typically prepared utilising the clostridial neurotoxin and a pyrogen-free sterile vehicle. The clostridial neurotoxin, depending on the vehicle and concentration used, can be either dissolved or suspended in the vehicle. In preparing solutions the clostridial neurotoxin can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, or high-pressure aerosol impingement.

The clostridial neurotoxin may be administered prior to, simultaneous or subsequent to the onset of brain tissue damage (e.g. neural tissue damage) caused by a brain disorder described herein. For example, where the brain disorder is caused by an infection, the clostridial neurotoxin may be administered subsequent to detection of the infection to promote neuroimmune response-mediated clearance of the infection.

In one embodiment, the clostridial neurotoxin may be administered prior to brain tissue damage (e.g. neural tissue damage). In other words, the clostridial neurotoxin may be administered before brain tissue damage (e.g. neural tissue damage) occurs.

Reference to “administering the clostridial neurotoxin before brain tissue damage (e.g. neural tissue damage) occurs” embraces the situation where brain tissue damage has previously occurred. For example, the clostridial neurotoxin may be administered to promote a neuroimmune response to treat or prevent further brain tissue damage. This is advantageous in the context of a brain disorder described herein, where (further) tissue damage can be anticipated.

In one embodiment, the clostridial neurotoxin is administered up to 4 days before brain tissue damage (e.g. neural tissue damage). The clostridial neurotoxin may be administered between 1-3 days before brain tissue damage (e.g. neural tissue damage) occurs. The clostridial neurotoxin may be administered 3 days before brain tissue damage (e.g. neural tissue damage). In a preferable embodiment, the clostridial neurotoxin may be administered ≤24 hours before brain tissue damage (e.g. neural tissue damage).

The inventors have demonstrated that administration of a clostridial neurotoxin may lead to a rapid promotion of the neuroimmune response. Thus, the clostridial neurotoxin may be administered at a time that brain tissue damage has occurred and/or is occurring (or is suspected to have occurred and/or be occurring) to promote the neuroimmune response to respond to the damage.

In one embodiment, the clostridial neurotoxin is administered within 48 hours (e.g. in less than or equal to 4 h hours) following the occurrence of brain tissue (e.g. neural tissue) damage. For example, the clostridial neurotoxin may be administered ≤10 minutes, ≤30 minutes, ≤1 hour, ≤2 hours, ≤5 hours, ≤12 hours, ≤34 hours, ≤3 h hours or ≤48 hours following the occurrence of brain tissue damage (e.g. neural tissue damage). Preferably, the clostridial neurotoxin may be administered ≤2 hours following the occurrence of brain tissue damage (e.g. neural tissue damage). More preferably, the clostridial neurotoxin may be administered within 1-10 minutes following the occurrence of brain tissue damage (e.g. neural tissue damage).

As explained in more detail elsewhere in this disclosure, the term “clostridial neurotoxin” embraces a clostridial neurotoxin fragment thereof.

Further information on clostridial neurotoxins, and suitable neurotoxins for use in the invention, is now provided below.

In one embodiment the present invention encompasses the use of full-length clostridial neurotoxins comprising a clostridial neurotoxin L-chain and a clostridial neurotoxin H-chain, optionally with the proviso that said clostridial neurotoxin L-chain is catalytically inactive.

The term “clostridial neurotoxin” embraces toxins produced by C. botulinum (botulinum neurotoxin serotypes A, B, C1, D, E, F, G, and X), C. tetani (tetanus neurotoxin), C. butyricum (botulinum neurotoxin serotype E), and C. baratii (botulinum neurotoxin serotype F), as well as modified clostridial neurotoxins or derivatives derived from any of the foregoing.

Botulinum neurotoxin (BoNT) is produced by C. botulinum in the form of a large protein complex, consisting of BoNT itself complexed to a number of accessory proteins. There are at present eight different classes of botulinum neurotoxin, namely: botulinum neurotoxin serotypes A, B, C1, D, E, F, G, and X all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1.

Tetanus toxin is produced in a single serotype by C. tetani. C. butyricum produces BoNT/E, while C. baratii produces BoNT/F.

The term “clostridial neurotoxin” is intended to encompass both full length neurotoxins (comprising a clostridial neurotoxin L-chain and a clostridial neurotoxin H-chain), as well as fragments thereof. For example, the “clostridial neurotoxin” may refer to a polypeptide that comprises or consists of a clostridial neurotoxin L-chain, a clostridial neurotoxin translocation domain (H_{N}) and/or a clostridial neurotoxin receptor binding domain (H_{C}) domain. The “clostridial neurotoxin” may refer to a polypeptide that comprises or consists of a clostridial neurotoxin L-chain, a clostridial neurotoxin translocation domain (H_{N}) and/or a clostridial neurotoxin receptor binding domain (H_{C}) domain, wherein when the polypeptide comprises a clostridial neurotoxin L-chain, the L-chain is a catalytically inactive.

Advantageously, the use of a clostridial neurotoxin fragment allows for the use of non-toxic (or substantially non-toxic) fragments of clostridial neurotoxins, which given the smaller size (compared to the full-length H-chain or full-length clostridial neurotoxin), are less likely to provoke an adverse immune response (against the fragment) in a subject administered said fragments. Moreover, the non-toxic (or substantially non-toxic) fragments are less expensive and/or less complex to manufacture than full-length clostridial neurotoxins. Additionally, the non-toxic (or substantially non-toxic) fragments constitute a more well-defined therapeutic than the full-length clostridial toxins, and given the shorter length of the polypeptides there is a reduced probability of, for example, cysteine shuffling between domains.

In embodiments which refer to a particular domain of a clostridial neurotoxin, the molecule may conveniently be referred to as a “polypeptide”. That is, the term “clostridial neurotoxin” may be substituted for the term “polypeptide”.

Thus, the clostridial neurotoxin referred to herein may be a polypeptide that comprises (or consists of) a clostridial neurotoxin light-chain (L-chain), a clostridial neurotoxin translocation domain (H_{N} domain) and/or a clostridial neurotoxin receptor binding domain (H_{C} domain). The clostridial neurotoxin referred to herein may be a polypeptide that comprises (or consists of) a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

It follows, therefore, that aspects of the invention provide any of the following:
a polypeptide for use in a method of treating a brain disorder (e.g. by promoting a neuroimmune response);
a method of treating a brain disorder in a patient (e.g. by promoting a neuroimmune response), the method comprising administering a polypeptide to the patient;
use of a polypeptide in the manufacture of a medicament to treat a brain disorder in a patient (e.g. by promoting a neuroimmune response);
wherein said peptide comprises (or consists of) a clostridial neurotoxin light-chain (L-chain), a clostridial neurotoxin translocation domain (H_{N} domain) and/or a clostridial neurotoxin receptor binding domain (H_{C} domain); optionally wherein when the polypeptide comprises a clostridial neurotoxin L-chain, the L-chain is catalytically inactive.

In one embodiment, a clostridial neurotoxin of the invention may be encoded by a nucleotide sequence having at least 70% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60. In one embodiment, a clostridial neurotoxin of the invention may be encoded by a nucleotide sequence having at least 80%, 90%, 95% or 98% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60. Preferably, a clostridial neurotoxin of the invention may be encoded by a nucleotide sequence comprising any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60.

In one embodiment a clostridial neurotoxin of the invention may comprise a clostridial neurotoxin sequence having at least 70% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65. In one embodiment a clostridial neurotoxin of the invention may comprise a clostridial neurotoxin sequence having at least 80%, 90%, 95% or 98% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65. Preferably, a clostridial neurotoxin of the invention may comprise a clostridial neurotoxin sequence of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65.

In one embodiment a clostridial neurotoxin of the invention may comprise a clostridial neurotoxin sequence having at least 70% sequence identity to any one of SEQ ID NOs: 51, 52, 53, 54, 55, 56, 57, 58, or 59 (preferably SEQ ID NOs: 51, 52, 53, 54, 55, 56, 57, or 59). In one embodiment a clostridial neurotoxin of the invention may comprise a clostridial neurotoxin sequence having at least 80%, 90%, 95% or 98% sequence identity to any one of SEQ ID NOs: 51, 52, 53, 54, 55, 56, 57, 58, or 59 (preferably SEQ ID NOs: 51, 52, 53, 54, 55, 56, 57, or 59). Preferably, a clostridial neurotoxin of the invention may comprise a clostridial neurotoxin sequence of any one of SEQ ID NOs: 51, 52, 53, 54, 55, 56, 57, 58, or 59 (preferably SEQ ID NOs: 51, 52, 53, 54, 55, 56, 57, or 59). In one embodiment a clostridial neurotoxin of the invention may comprise a clostridial neurotoxin sequence having at least 70% sequence identity to SEQ ID NO: 51. In one embodiment a clostridial neurotoxin of the invention may comprise a clostridial neurotoxin sequence having at least 80%, 90%, 95% or 98% sequence identity to SEQ ID NO: 51. Preferably, a clostridial neurotoxin of the invention may comprise a clostridial neurotoxin sequence of SEQ ID NO: 51.

The term “clostridial neurotoxin” is also intended to embrace modified clostridial neurotoxins and derivatives thereof, including but not limited to those described below. A modified clostridial neurotoxin or derivative may contain one or more amino acids that has been modified as compared to the native (unmodified) form of the clostridial neurotoxin, or may contain one or more inserted amino acids that are not present in the native (unmodified) form of the clostridial neurotoxin. By way of example, a modified clostridial neurotoxin may have modified amino acid sequences in one or more domains relative to the native (unmodified) clostridial neurotoxin sequence. Such modifications may modify functional aspects of the toxin, for example biological activity or persistence. Thus, in one embodiment, the clostridial neurotoxin of the invention is a modified clostridial neurotoxin, or a modified clostridial neurotoxin derivative, or a clostridial neurotoxin derivative.

A modified clostridial neurotoxin may have one or more modifications in the amino acid sequence of the heavy chain (such as a modified H_{C} domain), wherein said modified heavy chain binds to target nerve cells with a higher or lower affinity than the native (unmodified) clostridial neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

A modified clostridial neurotoxin may have one or more modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more modifications that increases or decreases the biological activity and/or the biological persistence of the modified clostridial neurotoxin. For example, a modified clostridial neurotoxin may comprise a leucine- or tyrosine-based motif, wherein said motif increases or decreases the biological activity and/or the biological persistence of the modified clostridial neurotoxin. Suitable leucine-based motifs include xDxxxLL, xExxxLL, xExxxIL, and xExxxLM (wherein x is any amino acid). Suitable tyrosine-based motifs include Y-x-x-Hy (wherein Hy is a hydrophobic amino acid). Examples of modified clostridial neurotoxins comprising leucine- and tyrosine-based motifs are described in WO 2002/08268, which is hereby incorporated by reference in its entirety.

As described above, a modified clostridial neurotoxin (or clostridial neurotoxin fragment) may be one that comprises one or more modifications that increases the isoelectric point of the clostridial neurotoxin when compared to an equivalent unmodified clostridial neurotoxin lacking said one or more modifications. Suitable modified clostridial neurotoxins are described above and in WO 2015/004461 A1 and WO 2016/110662 A1, which are incorporated herein by reference. Exemplary sequences include SEQ ID NOs: 61 and 42 described herein.

A modified clostridial neurotoxin may preferably comprise or consist of a sequence of SEQ ID No: 61.

The term “clostridial neurotoxin” is intended to embrace hybrid and chimeric clostridial neurotoxins. A hybrid clostridial neurotoxin comprises at least a portion of a light chain from one clostridial neurotoxin or subtype thereof, and at least a portion of a heavy chain from another clostridial neurotoxin or clostridial neurotoxin subtype. In one embodiment the hybrid clostridial neurotoxin may contain the entire light chain of a light chain from one clostridial neurotoxin subtype and the heavy chain from another clostridial neurotoxin subtype. In another embodiment, a chimeric clostridial neurotoxin may contain a portion (e.g. the binding domain) of the heavy chain of one clostridial neurotoxin subtype, with another portion of the heavy chain being from another clostridial neurotoxin subtype. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to subjects who are immunologically resistant to a given clostridial neurotoxin subtype, to subjects who may have a lower than average concentration of receptors to a given clostridial neurotoxin heavy chain binding domain, or to subjects who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety. Thus, in one embodiment, the clostridial neurotoxin (or fragment thereof) of the invention is a hybrid clostridial neurotoxin, or a chimeric clostridial neurotoxin.

In a particularly preferred embodiment, a polypeptide of the invention may be a chimeric clostridial neurotoxin comprising (preferably consisting of) a BoNT/A light-chain and translocation domain, and a BoNT/B receptor binding domain (H_{C} domain) or a portion thereof (e.g. BoNT/A LH_{N}-BoNT/B H_{C}). A suitable chimeric and/or hybrid clostridial neurotoxin may be one taught in WO 2017/191315 A1, which is incorporated herein by reference. Such preferred sequences include SEQ ID NOs: 44, 63, and 64.

For example, a chimeric clostridial neurotoxin may comprise (preferably consist of) a sequence of SEQ ID NO: 64 (which may be said to comprise a catalytically inactive L-chain).

In a preferable embodiment, a chimeric clostridial neurotoxin may comprise (preferably consist of) a sequence of SEQ ID NO: 63.

The BoNT/A LH_{N} domain may be covalently linked to the BoNT/B H_{C} domain. Said chimeric BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

Reference herein to the “first amino acid residue of the 3₁₀ helix separating the LH_{N} and H_{C} domains of BoNT/A” means the N-terminal residue of the 3₁₀ helix separating the LH_{N} and H_{C} domains.

Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureId=3BTA) and 1EPW (http://www.rcsb.org/pdb/explore/explore.do?structureId=1EPVV) for botulinum neurotoxins A1 and B1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular that the region around the “H_{N}/H_{CN}” junction is structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) was used to obtain a predicted structure of other BoNT serotypes based on the BoNT/A1 crystal structure (3BTA.pdb);
2. The structural (pdb) files thus obtained were edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) was used to superpose each serotype onto the 3BTA.pdb structure;
4. The superposed pdb files were inspected to locate the 3₁₀ helix at the start of the H_{C} domain of BoNT/A1, and corresponding residues in the other serotype were then identified;
5. The other BoNT serotype sequences were aligned with Clustal Omega in order to check that corresponding residues were correct.

Examples of LH_{N}, H_{C} and 3₁₀ helix domains determined by this method are presented below:

| | Accession Number | | | |
|---|---|---|---|---|
| | (Plus Sequence | | | |
| | Version after | | | |
| Neurotoxin | Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID | | | | |
| NO: 62) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (a.k.a. SEQ | | | | |
| ID NO: 52) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A2I2S2.1) | | | |
| BoNT/B4 | EF051570.1 (aka | 1-859 | 860-1291 | ⁸⁵⁹EILNNI⁸⁶⁴ |
| | A2I2W0.1) | | | |
| BoNT/B5 | EF033130.1 (aka | 1-859 | 860-1291 | ⁸⁵⁹DILNNI⁸⁶⁴ |
| | A2I2U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8^{th} residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a H_{C} domain from BoNT/B,
wherein the C-terminal amino acid residue of the LH_{N} domain corresponds to the eighth amino acid residue N-terminally to the β-strand located at the beginning (N-term) of the H_{C} domain of BoNT/A, and
wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a H_{C} domain from BoNT/B,
wherein the C-terminal amino acid residue of the LH_{N} domain corresponds to the C-terminal amino acid residue of the α-helix located at the end (C-term) of LH_{N} domain of BoNT/A, and
wherein the N-terminal amino acid residue of the H_{C} domain corresponds to the amino acid residue immediately C-terminal to the C-terminal amino acid residue of the α-helix located at the end (C-term) of LH_{N} domain of BoNT/B.

The rationale of the design process of the BoNT/AB chimera was to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the chimeric neurotoxin, thereby allowing for the chimeric neurotoxin to exert its functions to their full capacity.

The LH_{N} domain from BoNT/A may correspond to amino acid residues 1 to 872 of SEQ ID NO: 62, or a polypeptide sequence having at least 70% sequence identity thereto. The LH_{N} domain from BoNT/A may correspond to amino acid residues 1 to 872 of SEQ ID NO: 62, or a polypeptide sequence having at least 80%, 90% or 95% sequence identity thereto. Preferably, the LH_{N} domain from BoNT/A corresponds to amino acid residues 1 to 872 of SEQ ID NO: 62.

The H_{C} domain from BoNT/B may correspond to amino acid residues 860 to 1291 of SEQ ID NO: 52, or a polypeptide sequence having at least 70% sequence identity thereto. The H_{C} domain from BoNT/B may correspond to amino acid residues 860 to 1291 of SEQ ID NO: 52, or a polypeptide sequence having at least 80%, 90% or 95% sequence identity thereto. Preferably, the H_{C} domain from BoNT/B corresponds to amino acid residues 860 to 1291 of SEQ ID NO: 52.

Preferably, the BoNT/AB chimera comprises a BoNT/A LH_{N} domain and a BoNT/B H_{C} domain. More preferably, the LH_{N} domain corresponds to amino acid residues 1 to 872 of BoNT/A (SEQ ID NO: 62) and the H_{C} domain corresponds to amino acid residues 860 to 1291 of BoNT/B (SEQ ID NO: 52).

Preferably, a BoNT/B H_{C} domain further comprises at least one amino acid residue substitution, addition or deletion in the H_{CC} subdomain which has the effect of increasing the binding affinity of BoNT/B neurotoxin for human Syt II as compared to the natural BoNT/B sequence. Suitable amino acid residue substitution, addition or deletion in the BoNT/B H_{CC} subdomain have been disclosed in WO 2013/180799 and in WO 2016/154534 (both herein incorporated by reference).

Suitable amino acid residue substitution, addition or deletion in the BoNT/B H_{CC} subdomain include substitution mutations selected from the group consisting of: V1118M; Y1183M; E1191M; E1191I; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

Suitable amino acid residue substitution, addition or deletion in the BoNT/B H_{CC} subdomain further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

Suitable amino acid residue substitution, addition or deletion in the BoNT/B H_{CC} subdomain also include a combination of three substitution mutations which are E1191M, S1199W and W1178Q.

Preferably, the suitable amino acid residue substitution, addition or deletion in the BoNT/B H_{CC} subdomain includes a combination of two substitution mutations which are E1191M and S1199Y.

The modification may be a modification when compared to unmodified BoNT/B shown as SEQ ID NO: 52, wherein the amino acid residue numbering is determined by alignment with SEQ ID NO: 52. As the presence of a methionine residue at position 1 of SEQ ID NO: 52 is optional, the skilled person will take the presence/absence of the methionine residue into account when determining amino acid residue numbering. For example, where SEQ ID NO: 52 includes a methionine, the position numbering will be as defined above (e.g. E1191 will be E1191 of SEQ ID NO: 52). Alternatively, where the methionine is absent from SEQ ID NO: 52 the amino acid residue numbering should be modified by −1 (e.g. E1191 will be E1190 of SEQ ID NO: 52). Similar considerations apply when the methionine at position 1 of the other polypeptide sequences described herein is present/absent, and the skilled person will readily determine the correct amino acid residue numbering using techniques routine in the art.

Thus, in one aspect, the invention provides a clostridial neurotoxin for use in treating a brain disorder by promoting a neuroimmune response in a subject, wherein the clostridial neurotoxin comprises a polypeptide sequence having at least 70% sequence identity to SEQ ID NO: 63 or 64 (preferably wherein the clostridial neurotoxin comprises a polypeptide sequence having at least 70% sequence identity to SEQ ID NO: 63).

In a related aspect, there is provided a method for treating a brain disorder in a subject by promoting neuroimmune activity, the method comprising administering a clostridial neurotoxin to the subject, wherein the clostridial neurotoxin comprises a polypeptide sequence having at least 70% sequence identity to SEQ ID NO: 63 or 64 (preferably wherein the clostridial neurotoxin comprises a polypeptide sequence having at least 70% sequence identity to SEQ ID NO: 63).

In a further related aspect, there is provided use of a clostridial neurotoxin in the manufacture of a medicament for to treating a brain disorder in a subject by promoting a neuroimmune response, wherein the clostridial neurotoxin comprises a clostridial neurotoxin sequence having at least 70% sequence identity to SEQ ID NO: 63 or 64 (preferably wherein the clostridial neurotoxin comprises a polypeptide sequence having at least 70% sequence identity to SEQ ID NO: 63).

In one aspect the invention provides a clostridial neurotoxin for use in treating a brain disorder in a subject, wherein the clostridial neurotoxin comprises a polypeptide sequence having at least 70% sequence identity to SEQ ID NO: 63 or 64 (preferably wherein the clostridial neurotoxin comprises a polypeptide sequence having at least 70% sequence identity to SEQ ID NO: 63).

In a related aspect, there is provided a method for treating a brain disorder in a subject, the method comprising administering a clostridial neurotoxin to the subject, wherein the clostridial neurotoxin comprises a polypeptide sequence having at least 70% sequence identity to SEQ ID NO: 63 or 64 (preferably wherein the clostridial neurotoxin comprises a polypeptide sequence having at least 70% sequence identity to SEQ ID NO: 63).

In a further related aspect, there is provided use of a clostridial neurotoxin in the manufacture of a medicament for treating a brain disorder in a subject, wherein the clostridial neurotoxin comprises a polypeptide sequence having at least 70% sequence identity to SEQ ID NO: 63 or 64 (preferably wherein the clostridial neurotoxin comprises a polypeptide sequence having at least 70% sequence identity to SEQ ID NO: 63).

In one embodiment a clostridial neurotoxin for use according to the invention comprises a polypeptide sequence having at least 80%, 90%, 95% or 98% sequence identity to SEQ ID NO: 63 or 64. Preferably, a clostridial neurotoxin for use according to the invention comprises (more preferably consists of) a polypeptide sequence shown as SEQ ID NO: 63 or 64.

For example, a clostridial neurotoxin for use according to the invention may comprise a polypeptide sequence having at least 80%, 90%, 95% or 98% sequence identity to SEQ ID NO: 64. A clostridial neurotoxin may comprises (more preferably consist of) a polypeptide sequence shown as SEQ ID NO: 64.

In a preferred embodiment, a clostridial neurotoxin for use according to the invention may comprise a polypeptide sequence having at least 80%, 90%, 95% or 98% sequence identity to SEQ ID NO: 64. For example, a particularly suitable clostridial neurotoxin may comprises (more preferably consist of) a polypeptide sequence shown as SEQ ID NO: 63.

Preferably, the clostridial neurotoxin comprising a polypeptide sequence having at least 70% sequence identity to SEQ ID NO: 63 comprises a catalytically-inactive L-chain, such as SEQ ID NO: 64.

A chimeric and/or hybrid clostridial neurotoxin for use in the present invention may comprise a portion of a BoNT/A polypeptide and a portion of a BoNT/B polypeptide, an example of which includes the polypeptide described herein as SEQ ID NO: 44.

Suitable chimeric clostridial neurotoxins may include BoNT/FA. Indeed, in a particularly preferred embodiment, a clostridial neurotoxin (e.g. chimeric clostridial neurotoxin) of the invention may comprise BoNT/FA or a fragment thereof. Catalytically inactive forms of BoNT/FA are described herein as SEQ ID NO: 26 and 34. Suitable fragments of BoNT/FA are also described herein as SEQ ID NOs: 28, 30, and 32.

The term “clostridial neurotoxin” may also embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OTO22244.1), which cleaves VAMP2 and SNAP25, and the Chryseobacterium pipero encoded toxin (NCBI Ref.Seq: WP_034687872.1).

The clostridial neurotoxin of the present invention may lack a functional H_{C} domain of a clostridial neurotoxin and also lack any functionally equivalent exogenous ligand Targeting Moiety (TM).

Thus, in a particularly preferred embodiment, a clostridial neurotoxin of the invention is not a re-targeted clostridial neurotoxin. In a re-targeted clostridial neurotoxin, the clostridial neurotoxin is modified to include an exogenous ligand known as a Targeting Moiety (TM). The TM is selected to provide binding specificity for a desired target cell, and as part of the re-targeting process the native binding portion of the clostridial neurotoxin (e.g. the H_{C} domain, or the H_{CC} domain) may be removed. Re-targeting technology is described, for example, in: EP-B-0689459; WO 1994/021300; EP-B-0939818; U.S. Pat. Nos. 6,461,617; 7,192,596; WO 1998/007864; EP-B-0826051; U.S. Pat. Nos. 5,989,545; 6,395,513; 6,962,703; WO 1996/033273; EP-B-0996468; U.S. Pat. No. 7,052,702; WO 1999/017806; EP-B-1107794; U.S. Pat. No. 6,632,440; WO 2000/010598; WO 2001/21213; WO 2006/059093; WO 2000/62814; WO 2000/04926; WO 1993/15766; WO 2000/61192; and WO 1999/58571; all of which are hereby incorporated by reference in their entirety.

As discussed above, (full-length) clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

A clostridial neurotoxin may be selected from BoNT/A, BoNT/B, BoNT/C, BoNT/D, BoNT/E, BoNT/F, BoNT/G, BoNT/X, and TeNT (tetanus neurotoxin). Preferably, a clostridial neurotoxin is a botulinum neurotoxin, such as a botulinum neurotoxin selected from BoNT/A, BoNT/B, BoNT/C, BoNT/D, BoNT/E, BoNT/F, BoNT/G, and BoNT/X.

In one embodiment the clostridial neurotoxin may be BoNT/A. A reference BoNT/A sequence is shown as SEQ ID NO: 51. In another embodiment the clostridial neurotoxin may be BoNT/B. A reference BoNT/B sequence is shown as SEQ ID NO: 52. In another embodiment the clostridial neurotoxin may be BoNT/C. A reference BoNT/C sequence is shown as SEQ ID NO: 53. In another embodiment the clostridial neurotoxin may be BoNT/D. A reference BoNT/D sequence is shown as SEQ ID NO: 54. In another embodiment the clostridial neurotoxin may be BoNT/E. A reference BoNT/E sequence is shown as SEQ ID NO: 55. In another embodiment the clostridial neurotoxin may be BoNT/F. A reference BoNT/F sequence is shown as SEQ ID NO: 56. In another embodiment the clostridial neurotoxin may be BoNT/G. A reference BoNT/G sequence is shown as SEQ ID NO: 57. In another embodiment the clostridial neurotoxin may be TeNT. A reference TeNT sequence is shown as SEQ ID NO: 58. In another embodiment the clostridial neurotoxin may be BoNT/X. A reference BoNT/X sequence is shown as SEQ ID NO: 59.

In one embodiment a clostridial neurotoxin of the invention comprises a fragment of a BoNT/A or a fragment of a BoNT/F. In another embodiment, the clostridial neurotoxin of the invention comprises a catalytically inactive L-chain of BoNT/A or BoNT/F. For example, the clostridial neurotoxin of the invention may comprise a catalytically inactive L-chain of BoNT/A.

In embodiments where a clostridial neurotoxin described herein has a tag for purification (e.g. a His-tag) and/or a linker, said tag and/or linker are optional.

Suitable full-length clostridial neurotoxins are described herein.

In one embodiment a clostridial neurotoxin of the invention may comprise a polypeptide sequence having at least 70% sequence identity to any one of SEQ ID NOs: 2, 10, 12, 14, 16, 18, 26, 34, 51, 52, 53, 54, 55, 56, 57, 58, 59, 61, 62, 63, 64 or 65, preferably with the proviso that a clostridial neurotoxin L-chain of said clostridial neurotoxin is catalytically inactive. In one embodiment a clostridial neurotoxin of the invention may comprise a polypeptide sequence having at least 80%, 90%, 95% or 98% sequence identity to any one of SEQ ID NOs: 2, 10, 12, 14, 16, 18, 26, 34, 51, 52, 53, 54, 55, 56, 57, 58, 59, 61, 62, 63, 64 or 65, preferably with the proviso that a clostridial neurotoxin L-chain of said clostridial neurotoxin is catalytically inactive. Preferably, a clostridial neurotoxin of the invention may comprise a polypeptide sequence comprising any one of SEQ ID NOs: 2, 10, 12, 14, 16, 18, 26, 34, 51, 52, 53, 54, 55, 56, 57, 58, 59, 61, 62, 63, 64 or 65, preferably with the proviso that a clostridial neurotoxin L-chain of said clostridial neurotoxin is catalytically inactive.

In one embodiment a clostridial neurotoxin of the invention may be one encoded by a nucleotide sequence having at least 70% sequence identity to any one of SEQ ID NOs: 1, 9, 11, 13, 15, 17, 25, 33, or 60, preferably with the proviso that the clostridial neurotoxin L-chain of said clostridial neurotoxin is catalytically inactive. In one embodiment a clostridial neurotoxin of the invention is one encoded by a nucleotide sequence having at least 80%, 90%, 95% or 98% sequence identity to any one of SEQ ID NOs: 1, 9, 11, 13, 15, 17, 25, 33, or 60, preferably with the proviso that the clostridial neurotoxin L-chain of said clostridial neurotoxin is catalytically inactive. Preferably, a clostridial neurotoxin of the invention is one encoded by a nucleotide sequence comprising any one of SEQ ID NOs: 1, 9, 11, 13, 15, 17, 25, 33, or 60, preferably with the proviso that the clostridial neurotoxin L-chain of said clostridial neurotoxin is catalytically inactive.

In one embodiment a clostridial neurotoxin of the invention may comprise a polypeptide sequence having at least 70% sequence identity to any one of SEQ ID NOs: 2, 10, 12, 14, 16, 18, 26, 34, 64 or 65, preferably with the proviso that the clostridial neurotoxin L-chain of said clostridial neurotoxin is catalytically inactive. In one embodiment a clostridial neurotoxin of the invention comprises a polypeptide sequence having at least 80%, 90%, 95% or 98% sequence identity to any one of SEQ ID NOs: 2, 10, 12, 14, 16, 18, 26, 34, 64 or 65, preferably with the proviso that the clostridial neurotoxin L-chain of said clostridial neurotoxin is catalytically inactive. Preferably, a clostridial neurotoxin of the invention comprises any one of SEQ ID NOs: 2, 10, 12, 14, 16, 18, 26, 34, 64 or 65, preferably with the proviso that the clostridial neurotoxin L-chain of said clostridial neurotoxin is catalytically inactive.

In one embodiment a clostridial neurotoxin of the invention is a full-length clostridial neurotoxin selected from BoNT/A, BoNT/B, BoNT/C, BoNT/D, BoNT/E, BoNT/F, BoNT/G, BoNT/X, and TeNT.

In one embodiment a clostridial neurotoxin of the invention is a full-length clostridial neurotoxin selected from BoNT/B, BoNT/C, BoNT/D, BoNT/E, BoNT/F, BoNT/G, BoNT/X, and TeNT.

In one embodiment a clostridial neurotoxin of the invention may comprise a polypeptide sequence having at least 70% sequence identity to any one of SEQ ID NOs: 51-59, 61 or 63. In one embodiment a clostridial neurotoxin of the invention may comprise a polypeptide sequence having at least 80%, 90%, 95% or 98% sequence identity to any one of SEQ ID NOs: 51-59, 61 or 63. In one embodiment a clostridial neurotoxin of the invention may comprise a polypeptide sequence having at least 99% or 99.9% sequence identity to any one of SEQ ID NOs: 51-59, 61 or 63. Preferably, a clostridial neurotoxin of the invention may comprise (more preferably consist of) a polypeptide sequence comprising any one of SEQ ID NOs: 51-59, 61 or 63.

In one embodiment a clostridial neurotoxin of the invention may comprise a polypeptide sequence having at least 70% sequence identity to any one of SEQ ID NOs: 52-59, 61 or 63. In one embodiment a clostridial neurotoxin of the invention may comprise a polypeptide sequence having at least 80%, 90%, 95% or 98% sequence identity to any one of SEQ ID NOs: 52-59, 61 or 63. In one embodiment a clostridial neurotoxin of the invention may comprise a polypeptide sequence having at least 99% or 99.9% sequence identity to any one of SEQ ID NOs: 52-59, 61 or 63. Preferably, a clostridial neurotoxin of the invention may comprise (more preferably consist of) a polypeptide sequence comprising any one of SEQ ID NOs: 52-59, 61 or 63.

In one embodiment a clostridial neurotoxin of the invention is not a full-length catalytically active clostridial neurotoxin, e.g. is not full-length catalytically active BoNT/A.

The clostridial neurotoxin of the present invention may comprise (or consist of) a fragment of a clostridial neurotoxin, e.g. a fragment of any full-length clostridial neurotoxin described herein.

In one embodiment a clostridial neurotoxin of the invention may comprise a fragment of a polypeptide sequence having at least 70% sequence identity to any one of SEQ ID NOs: 2, 10, 12, 14, 16, 18, 26, 34, 51, 52, 53, 54, 55, 56, 57, 58, 59, 61, 62, 63, 64 or 65. In one embodiment a clostridial neurotoxin of the invention may comprise a fragment of a polypeptide sequence having at least 80%, 90%, 95% or 98% sequence identity to any one of SEQ ID NOs: 2, 10, 12, 14, 16, 18, 26, 34, 51, 52, 53, 54, 55, 56, 57, 58, 59, 61, 62, 63, 64 or 65. Preferably, a clostridial neurotoxin of the invention may comprise a fragment of a polypeptide sequence comprising any one of SEQ ID NOs: 2, 10, 12, 14, 16, 18, 26, 34, 51, 52, 53, 54, 55, 56, 57, 58, 59, 61, 62, 63, 64 or 65.

In one embodiment a clostridial neurotoxin of the invention comprises (or consists of) a clostridial neurotoxin L-chain or fragment thereof. A fragment of a clostridial neurotoxin L-chain may have 400, 350, 300, 250, 200, 100 or 50 amino acid residues of a clostridial neurotoxin L-chain. In one embodiment, a fragment of a clostridial neurotoxin L-chain has at least 20, 30, 40, 50, 60, 70, 80, 90, 100, 120, 150 or 200 amino acid residues of a clostridial neurotoxin L-chain. For example, a fragment of a clostridial neurotoxin L-chain may have 20-400, 50-300 or 100-200 amino acid residues of a clostridial neurotoxin L-chain.

Examples of L-chain reference sequences include:
Botulinum type A neurotoxin: amino acid residues 1-448
Botulinum type B neurotoxin: amino acid residues 1-440
Botulinum type C1 neurotoxin: amino acid residues 1-441
Botulinum type D neurotoxin: amino acid residues 1-445
Botulinum type E neurotoxin: amino acid residues 1-422
Botulinum type F neurotoxin: amino acid residues 1-439
Botulinum type G neurotoxin: amino acid residues 1-441
Tetanus neurotoxin: amino acid residues 1-457

For recently-identified BoNT/X, the L-chain has been reported as corresponding to amino acids 1-439 thereof, with the L-chain boundary potentially varying by approximately 25 amino acids (e.g. 1-414 or 1-464).

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441
Botulinum type C1 neurotoxin: amino acid residues M1-K449
Botulinum type D neurotoxin: amino acid residues M1-R445
Botulinum type E neurotoxin: amino acid residues M1-R422
Botulinum type F neurotoxin: amino acid residues M1-K439
Botulinum type G neurotoxin: amino acid residues M1-K446
Tetanus neurotoxin: amino acid residues M1-A457

Suitable clostridial neurotoxin L-chains are described herein.

A clostridial neurotoxin L-chain may comprise a polypeptide sequence having at least 70% sequence identity to any one of SEQ ID NOs: 6, 24, 32 or 40 or a fragment thereof. In one embodiment a clostridial neurotoxin L-chain comprises a polypeptide sequence having at least 80%, 90%, 95% or 98% sequence identity to any one of SEQ ID NOs: 6, 24, 32 or 40 or a fragment thereof. Preferably, a clostridial neurotoxin L-chain comprises (more preferably consists of) a polypeptide sequence comprising any one of SEQ ID NOs: 6, 24, 32 or 40 or a fragment thereof.

A clostridial neurotoxin L-chain may be one encoded by a nucleotide sequence having at least 70% sequence identity to any one of SEQ ID NOs: 5, 23, 31 or 39 or a fragment thereof. In one embodiment a clostridial neurotoxin L-chain is one encoded by a nucleotide sequence having at least 80%, 90%, 95% or 98% sequence identity to any one of SEQ ID NOs: 5, 23, 31 or 39 or a fragment thereof. Preferably, a clostridial neurotoxin L-chain is one encoded by a nucleotide sequence comprising any one of SEQ ID NOs: 5, 23, 31 or 39 or a fragment thereof.

In one embodiment a clostridial neurotoxin of the invention comprises (or consists of) a fragment of a clostridial neurotoxin H-chain. A fragment of a clostridial neurotoxin H-chain may have ≤300, ≤700, ≤600, ≤500, ≤350, ≤300, ≤250, ≤200, ≤100 or ≤50 amino acid residues of a clostridial neurotoxin H-chain. In one embodiment, a fragment of a clostridial neurotoxin H-chain has at least 20, 30, 40, 50, 60, 70, 80, 90, 100, 120, 150 or 200 amino acid residues of a clostridial neurotoxin H-chain. For example, a fragment of a clostridial neurotoxin H-chain may have 20-800, 30-600, 40-400, 50-300 or 100-200 amino acid residues of a clostridial neurotoxin H-chain.

A clostridial neurotoxin H-chain comprises two structural/functional domains: the translocation domain (H_{N}) and receptor binding domain (H_{C}).

In one embodiment a clostridial neurotoxin of the invention comprises (or consists of) a clostridial neurotoxin translocation domain or a fragment thereof. A fragment of a clostridial neurotoxin translocation domain may have ≤400, ≤350, ≤300, ≤250, ≤200, ≤100 or ≤50 amino acid residues of a clostridial neurotoxin translocation domain. In one embodiment, a fragment of a clostridial neurotoxin translocation domain has at least 20, 30, 40, 50, 60, 70, 80, 90, 100, 120, 150 or 200 amino acid residues of a clostridial neurotoxin translocation domain. For example, a fragment of a clostridial neurotoxin translocation domain may have 20-400, 50-300 or 100-200 amino acid residues of a clostridial neurotoxin translocation domain.

The translocation domain is a fragment of the H-chain of a clostridial neurotoxin approximately equivalent to the amino-terminal half of the H-chain, or the domain corresponding to that fragment in the intact H-chain. In one embodiment the H_{C} function of the H-chain may be removed by deletion of the H_{C} amino acid sequence (either at the DNA synthesis level, or at the post-synthesis level by nuclease or protease treatment). Alternatively, the H_{C} function may be inactivated by chemical or biological treatment. Thus, in some embodiments the H-chain may be incapable of binding to the Binding Site on a target cell to which native clostridial neurotoxin (i.e. holotoxin) binds.

Examples of suitable (reference) Translocation Domains include:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)
Botulinum type C neurotoxin—amino acid residues (T450-N866)
Botulinum type D neurotoxin—amino acid residues (D446-N862)
Botulinum type E neurotoxin—amino acid residues (K423-K845)
Botulinum type F neurotoxin—amino acid residues (A440-K864)
Botulinum type G neurotoxin—amino acid residues (S447-S863)
Tetanus neurotoxin—amino acid residues (5458-V879)

In the context of the present invention, a variety of clostridial neurotoxin H_{N} regions comprising a translocation domain can be useful in aspects of the present invention. In one embodiment these active fragments can facilitate the release of a non-cytotoxic protease (e.g. a clostridial L-chain) from intracellular vesicles into the cytoplasm of the target cell and thus participate in executing the overall cellular mechanism whereby a clostridial neurotoxin proteolytically cleaves a substrate. The H_{N} regions from the heavy chains of clostridial neurotoxins are approximately 410-430 amino acids in length and comprise a translocation domain. Research has shown that the entire length of a H_{N} region from a clostridial neurotoxin heavy chain is not necessary for the translocating activity of the translocation domain. Thus, aspects of this embodiment can include clostridial neurotoxin H_{N} regions comprising a translocation domain having a length of, for example, at least 350 amino acids, at least 375 amino acids, at least 400 amino acids and at least 425 amino acids. Other aspects of this embodiment can include clostridial neurotoxin H_{N} regions comprising a translocation domain having a length of, for example, at most 350 amino acids, at most 375 amino acids, at most 400 amino acids and at most 425 amino acids.

For further details on the genetic basis of toxin production in Clostridium botulinum and C. tetani, see Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues. In one embodiment said modified H_{N} portions still demonstrate the above-mentioned translocation function.

In a preferred embodiment a clostridial neurotoxin of the invention comprises (or consists of) a clostridial neurotoxin receptor binding domain (H_{C}) or a fragment thereof. A fragment of a clostridial neurotoxin receptor binding domain (H_{C}) may have ≤350, ≤300, ≤250, ≤200, ≤100 or ≤50 amino acid residues of a clostridial neurotoxin receptor binding domain (H_{C}). In one embodiment, a fragment of a clostridial neurotoxin receptor binding domain (H_{C}) has at least 20, 30, 40, 50, 60, 70, 80, 90, 100, 120, 150 or 200 amino acid residues of a clostridial neurotoxin receptor binding domain (H_{C}). For example, a fragment of a clostridial neurotoxin receptor binding domain (H_{C}) may have 20-350, 50-300 or 100-200 amino acid residues of a clostridial neurotoxin receptor binding domain (H_{C}).

Examples of clostridial neurotoxin receptor binding domain (H_{C}) reference sequences include:
BoNT/A—N872-L1296
BoNT/B—E859-E1291
BoNT/C1—N867-E1291
BoNT/D—S863-E1276
BoNT/E—R846-K1252
BoNT/F—K865-E1274
BoNT/G—N864-E1297
TeNT—1880-D1315

For recently-identified BoNT/X, the H_{C} domain has been reported as corresponding to amino acids 893-1306 thereof, with the domain boundary potentially varying by approximately 25 amino acids (e.g. 868-1306 or 918-1306).

A clostridial neurotoxin H-chain may further comprise a translocation facilitating domain. Said domain facilitates delivery of the L-chain into the cytosol of the target cell and are described, for example, in WO 08/008803 and WO 08/008805, each of which is herein incorporated by reference thereto.

By way of example, a translocation facilitating domain may comprise a clostridial neurotoxin H_{CN} domain or a fragment or variant thereof. In more detail, a clostridial neurotoxin H_{CN} translocation facilitating domain may have a length of at least 200 amino acids, at least 225 amino acids, at least 250 amino acids, at least 275 amino acids. In this regard, a clostridial neurotoxin H_{CN} translocation facilitating domain preferably has a length of at most 200 amino acids, at most 225 amino acids, at most 250 amino acids, or at most 275 amino acids. Specific (reference) examples include:
Botulinum type A neurotoxin—amino acid residues (872-1110)
Botulinum type B neurotoxin—amino acid residues (859-1097)
Botulinum type C neurotoxin—amino acid residues (867-1111)
Botulinum type D neurotoxin—amino acid residues (863-1098)
Botulinum type E neurotoxin—amino acid residues (846-1085)
Botulinum type F neurotoxin—amino acid residues (865-1105)
Botulinum type G neurotoxin—amino acid residues (864-1105)
Tetanus neurotoxin—amino acid residues (880-1127)

The above sequence positions may vary a little according to serotype/sub-type, and further examples of suitable (reference) clostridial neurotoxin H_{CN} domains include:
Botulinum type A neurotoxin—amino acid residues (874-1110)
Botulinum type B neurotoxin—amino acid residues (861-1097)
Botulinum type C neurotoxin—amino acid residues (869-1111)
Botulinum type D neurotoxin—amino acid residues (865-1098)
Botulinum type E neurotoxin—amino acid residues (848-1085)
Botulinum type F neurotoxin—amino acid residues (867-1105)
Botulinum type G neurotoxin—amino acid residues (866-1105)
Tetanus neurotoxin—amino acid residues (882-1127)

Suitable clostridial neurotoxin H_{C} domains are described herein.

A clostridial neurotoxin H_{C} domain may comprise a polypeptide sequence having at least 70% sequence identity to any one of SEQ ID NOs: 8, 22, 30, 38, 42, 44, 46, 48 or 50 or a fragment thereof. In one embodiment a clostridial neurotoxin H_{C} domain comprises a polypeptide sequence having at least 80%, 90%, 95% or 98% sequence identity to any one of SEQ ID NOs: 8, 22, 30, 38, 42, 44, 46, 48 or 50 or a fragment thereof. Preferably, a clostridial neurotoxin H_{C} domain comprises (more preferably consists of) a polypeptide sequence comprising any one of SEQ ID NOs: 8, 22, 30, 38, 42, 44, 46, 48 or 50 or a fragment thereof.

A clostridial neurotoxin H_{C} domain may be one encoded by a nucleotide sequence having at least 70% sequence identity to any one of SEQ ID NOs: 7, 21, 29, 37, 41, 43, 45, 47 or 49 or a fragment thereof. In one embodiment a clostridial neurotoxin H_{C} domain is one encoded by a nucleotide sequence having at least 80%, 90%, 95% or 98% sequence identity to any one of SEQ ID NOs: 7, 21, 29, 37, 41, 43, 45, 47 or 49 or a fragment thereof. Preferably, a clostridial neurotoxin H_{C} domain is one encoded by a nucleotide sequence comprising any one of SEQ ID NOs: 7, 21, 29, 37, 41, 43, 45, 47 or 49 or a fragment thereof.

In one embodiment a clostridial neurotoxin H_{C} domain for use in the invention is a variant BoNT/A H_{C} domain. Said variant BoNT/A H_{C} domain may comprise a modification of one or more amino acids residues selected from Y1117, F1252, H1253, and L1278. For example, a variant BoNT/A H_{C} domain may comprise one or more (preferably two or more) of the following modifications Y1117V, F1252Y, H1253K, and L1278F or L1278H.

In one embodiment a variant BoNT/A H_{C} domain comprises the following modifications: Y1117V and H1253K; or Y1117V, F1252Y, H1253K, and L1278F; or Y1117V, F1252Y, H1253K, and L1278H.

Preferably, a variant BoNT/A H_{C} domain comprises the following modifications: Y1117V and H1253K; or Y1117V, F1252Y, H1253K, and L1278H.

The modification may be a modification when compared to unmodified BoNT/A shown as SEQ ID NO: 62, wherein the amino acid residue numbering is determined by alignment with SEQ ID NO: 62. As the presence of a methionine residue at position 1 of SEQ ID NO: 62 is optional, the skilled person will take the presence/absence of the methionine residue into account when determining amino acid residue numbering. For example, where SEQ ID NO: 62 includes a methionine, the position numbering will be as defined above (e.g. Y1117 will align against Y1117 of SEQ ID NO: 62). Alternatively, where the methionine is absent from SEQ ID NO: 62 the amino acid residue numbering should be modified by −1 (e.g. Y1117 will align against Y1116 of SEQ ID NO: 52). Similar considerations apply when the methionine at position 1 of the other polypeptide sequences described herein is present/absent, and the skilled person will readily determine the correct amino acid residue numbering using techniques routine in the art.

A variant BoNT/A H_{C} domain may comprise a polypeptide sequence having at least 70% sequence identity to any one of SEQ ID NOs: 46, 48 or 50 or a fragment thereof with the proviso that the variant BoNT/A H_{C} domain comprises a modification as described above. In one embodiment a variant BoNT/A H_{C} domain comprises a polypeptide sequence having at least 80%, 90%, 95% or 98% sequence identity to any one of SEQ ID NOs: 46, 48 or 50 or a fragment thereof with the proviso that the variant BoNT/A H_{C} domain comprises a modification as described above. In one embodiment a variant BoNT/A H_{C} domain comprises a polypeptide sequence having at least 99% or 99.9% sequence identity to any one of SEQ ID NOs: 46, 48 or 50 or a fragment thereof with the proviso that the variant BoNT/A H_{C} domain comprises a modification as described above. Preferably, a variant BoNT/A H_{C} domain comprises (more preferably consists of) a polypeptide sequence comprising any one of SEQ ID NOs: 46, 48 or 50 or a fragment thereof.

A variant BoNT/A H_{C} domain may comprise a polypeptide sequence having at least 70% sequence identity to any one of SEQ ID NOs: 46 or 50 or a fragment thereof with the proviso that the variant BoNT/A H_{C} domain comprises a modification as described above. In one embodiment a variant BoNT/A H_{C} domain comprises a polypeptide sequence having at least 80%, 90%, 95% or 98% sequence identity to any one of SEQ ID NOs: 46 or 50 or a fragment thereof with the proviso that the variant BoNT/A H_{C} domain comprises a modification as described above. In one embodiment a variant BoNT/A H_{C} domain comprises a polypeptide sequence having at least 99% or 99.9% sequence identity to any one of SEQ ID NOs: 46 or 50 or a fragment thereof with the proviso that the variant BoNT/A H_{C} domain comprises a modification as described above. Preferably, a variant BoNT/A H_{C} domain comprises (more preferably consists of) a polypeptide sequence comprising any one of SEQ ID NOs: 46 or 50 or a fragment thereof.

A variant BoNT/A H_{C} domain may be one encoded by a nucleotide sequence having at least 70% sequence identity to any one of SEQ ID NOs: 45, 47 or 49 or a fragment thereof with the proviso that the variant BoNT/A H_{C} domain comprises a modification as described above. In one embodiment a variant BoNT/A H_{C} domain be one encoded by a nucleotide sequence having at least 80%, 90%, 95% or 98% sequence identity to any one of SEQ ID NOs: 45, 47 or 49 or a fragment thereof with the proviso that the variant BoNT/A H_{C} domain comprises a modification as described above. In one embodiment a variant BoNT/A H_{C} domain be one encoded by a nucleotide sequence having at least 99% or 99.9% sequence identity to any one of SEQ ID NOs: 45, 47 or 49 or a fragment thereof with the proviso that the variant BoNT/A H_{C} domain comprises a modification as described above. Preferably, a variant BoNT/A H_{C} domain be one encoded by any one of SEQ ID NOs: 45, 47 or 49 or a fragment thereof.

A variant BoNT/A H_{C} domain may be one encoded by a nucleotide sequence having at least 70% sequence identity to any one of SEQ ID NOs: 45 or 49 or a fragment thereof with the proviso that the variant BoNT/A H_{C} domain comprises a modification as described above. In one embodiment a variant BoNT/A H_{C} domain be one encoded by a nucleotide sequence having at least 80%, 90%, 95% or 98% sequence identity to any one of SEQ ID NOs: 45 or 49 or a fragment thereof with the proviso that the variant BoNT/A H_{C} domain comprises a modification as described above. In one embodiment a variant BoNT/A H_{C} domain be one encoded by a nucleotide sequence having at least 99% or 99.9% sequence identity to any one of SEQ ID NOs: 45 or 49 or a fragment thereof with the proviso that the variant BoNT/A H_{C} domain comprises a modification as described above. Preferably, a variant BoNT/A H_{C} domain be one encoded by any one of SEQ ID NOs: 45 or 49 or a fragment thereof.

Any of the above-described facilitating domains may be combined with any of the previously described translocation domain peptides that are suitable for use in the present invention. Thus, by way of example, a non-clostridial facilitating domain may be combined with non-clostridial translocation domain peptide or with clostridial translocation domain peptide. Alternatively, a clostridial neurotoxin H_{CN} translocation facilitating domain may be combined with a non-clostridial translocation domain peptide. Alternatively, a clostridial neurotoxin H_{CN} facilitating domain may be combined with a clostridial translocation domain peptide, examples of which include:
Botulinum type A neurotoxin—amino acid residues (449-1110)
Botulinum type B neurotoxin—amino acid residues (442-1097)
Botulinum type C neurotoxin—amino acid residues (450-1111)
Botulinum type D neurotoxin—amino acid residues (446-1098)
Botulinum type E neurotoxin—amino acid residues (423-1085)
Botulinum type F neurotoxin—amino acid residues (440-1105)
Botulinum type G neurotoxin—amino acid residues (447-1105)
Tetanus neurotoxin—amino acid residues (458-1127)

In some embodiments the clostridial neurotoxins of the present invention may lack a functional H_{C} domain of a clostridial neurotoxin. In one embodiment, the clostridial neurotoxins preferably lack the last 50 C-terminal amino acids of a clostridial neurotoxin holotoxin. In another embodiment, the clostridial neurotoxins preferably lack the last 100, preferably the last 150, more preferably the last 200, particularly preferably the last 250, and most preferably the last 300 C-terminal amino acid residues of a clostridial neurotoxin holotoxin. Alternatively, the H_{C} binding activity may be negated/reduced by mutagenesis—by way of example, referring to BoNT/A for convenience, modification of one or two amino acid residue mutations (W1266 to L and Y1267 to F) in the ganglioside binding pocket causes the H_{C} region to lose its receptor binding function. Analogous mutations may be made to non-serotype A clostridial peptide components, e.g. a construct based on botulinum B with mutations (W1262 to L and Y1263 to F) or botulinum E (W1224 to L and Y1225 to F). Other mutations to the active site achieve the same ablation of H_{C} receptor binding activity, e.g. Y1267S in botulinum type A toxin and the corresponding highly conserved residue in the other clostridial neurotoxins. Details of this and other mutations are described in Rummel et al (2004) (Molecular Microbiol. 51:631-634), which is hereby incorporated by reference thereto.

The H_{C} peptide of a native clostridial neurotoxin comprises approximately 400-440 amino acid residues, and consists of two functionally distinct domains of approximately 25 kDa each, namely the N-terminal region (commonly referred to as the H_{N} peptide or domain) and the C-terminal region (commonly referred to as the H_{CC} peptide or domain). This fact is confirmed by the following publications, each of which is herein incorporated in its entirety by reference thereto: Umland T C (1997) Nat. Struct. Biol. 4: 788-792; Herreros J (2000) Biochem. J. 347: 199-204; Halpern J (1993) J. Biol. Chem. 268: 15, pp. 11188-11192; Rummel A (2007) PNAS 104: 359-364; Lacey D B (1998) Nat. Struct. Biol. 5: 898-902; Knapp (1998) Am. Cryst. Assoc. Abstract Papers 25: 90; Swaminathan and Eswaramoorthy (2000) Nat. Struct. Biol. 7: 1751-1759; and Rummel A (2004) Mol. Microbiol. 51(3), 631-643. Moreover, it has been well documented that the C-terminal region (H_{CC}), which constitutes the C-terminal 160-200 amino acid residues, is responsible for binding of a clostridial neurotoxin to its natural cell receptors, namely to nerve terminals at the neuromuscular junction—this fact is also confirmed by the above publications. Thus, reference throughout this specification to a clostridial heavy-chain lacking a functional heavy chain H_{C} peptide (or domain) such that the heavy-chain is incapable of binding to cell surface receptors to which a native clostridial neurotoxin binds means that the clostridial heavy-chain simply lacks a functional H_{CC} peptide. In other words, the H_{CC} peptide region may be either partially or wholly deleted, or otherwise modified (e.g. through conventional chemical or proteolytic treatment) to reduce its native binding ability for nerve terminals at the neuromuscular junction.

Thus, in one embodiment, a clostridial neurotoxin H_{N} peptide of the present invention lacks part of a C-terminal peptide portion (H_{CC}) of a clostridial neurotoxin and thus lacks the H_{C} binding function of native clostridial neurotoxin. By way of example, in one embodiment, the C-terminally extended clostridial H_{N} peptide lacks the C-terminal 40 amino acid residues, or the C-terminal 60 amino acid residues, or the C-terminal 80 amino acid residues, or the C-terminal 100 amino acid residues, or the C-terminal 120 amino acid residues, or the C-terminal 140 amino acid residues, or the C-terminal 150 amino acid residues, or the C-terminal 160 amino acid residues of a clostridial neurotoxin heavy-chain. In another embodiment, the clostridial H_{N} peptide of the present invention lacks the entire C-terminal peptide portion (H_{CC}) of a clostridial neurotoxin and thus lacks the H_{C} binding function of native clostridial neurotoxin. By way of example, in one embodiment, the clostridial H_{N} peptide lacks the C-terminal 165 amino acid residues, or the C-terminal 170 amino acid residues, or the C-terminal 175 amino acid residues, or the C-terminal 180 amino acid residues, or the C-terminal 185 amino acid residues, or the C-terminal 190 amino acid residues, or the C-terminal 195 amino acid residues of a clostridial neurotoxin heavy-chain. By way of further example, the clostridial H_{N} peptide of the present invention lacks a clostridial H_{CC} reference sequence selected from the group consisting of:
Botulinum type A neurotoxin—amino acid residues (Y1111-L1296)
Botulinum type B neurotoxin—amino acid residues (Y1098-E1291)
Botulinum type C neurotoxin—amino acid residues (Y1112-E1291)
Botulinum type D neurotoxin—amino acid residues (Y1099-E1276)
Botulinum type E neurotoxin—amino acid residues (Y1086-K1252)
Botulinum type F neurotoxin—amino acid residues (Y1106-E1274)
Botulinum type G neurotoxin—amino acid residues (Y1106-E1297)
Tetanus neurotoxin—amino acid residues (Y1128-D1315).

The above-identified reference sequences should be considered a guide as slight variations may occur according to sub-serotypes.

In a preferred embodiment a clostridial neurotoxin of the invention comprises (or consists of) a clostridial neurotoxin L-chain or fragment thereof and a fragment of a clostridial neurotoxin H-chain. For example, a clostridial neurotoxin may comprise (or consist of) a clostridial neurotoxin L-chain or fragment thereof and a clostridial neurotoxin translocation domain (H_{N}). Preferably, the clostridial neurotoxin does not further comprise a clostridial neurotoxin receptor binding domain (H_{C}) or at least the C-terminal portion of a clostridial neurotoxin receptor binding domain (H_{CC}). Thus, in one embodiment a clostridial neurotoxin of the present invention lacks a C-terminal portion of a clostridial neurotoxin receptor binding domain (H_{CC}). Advantageously, such clostridial neurotoxins lack the endogenous clostridial neurotoxin receptor binding capabilities and thus exhibit fewer off-target effects in a subject administered said clostridial neurotoxin.

In one embodiment a clostridial neurotoxin of the invention consists essentially of a clostridial neurotoxin L-chain or fragment thereof and/or a fragment of a clostridial neurotoxin H-chain. The term “consists essentially of” as used in this context means that the clostridial neurotoxin does not further comprise one or more amino acid residues that confer additional functionality to the clostridial neurotoxin, e.g. when administered to a subject. In other words, a clostridial neurotoxin that “consists essentially of” a clostridial neurotoxin L-chain or fragment thereof and/or a fragment of a clostridial neurotoxin H-chain may further comprise one or more amino acid residues (to those of the clostridial neurotoxin L-chain or fragment thereof and/or fragment of a clostridial neurotoxin H-chain) but said one or more further amino acid residues do not confer additional functionality to the clostridial neurotoxin, e.g. when administered to a subject. Additional functionality may include enzymatic activity, binding activity and/or any physiological activity whatsoever.

In one embodiment a clostridial neurotoxin may comprise non-clostridial neurotoxin sequences in addition to any clostridial neurotoxin sequences. The non-clostridial neurotoxin sequences preferably do not disrupt the ability of a clostridial neurotoxin of the invention to promote a neuroimmune response. Preferably, the non-clostridial neurotoxin sequence is not one having catalytic activity, e.g. enzymatic activity. Preferably, the non-clostridial sequence is not one that binds to a cellular receptor. In other words, it is most preferred that the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp#db_reports. Non-clostridial neurotoxin sequences may include tags to aid in purification, such as His-tags. It is preferred that any clostridial neurotoxin sequences comprised in said clostridial neurotoxin consist of a clostridial neurotoxin L-chain or fragment thereof and/or a fragment of a clostridial neurotoxin H-chain. In one embodiment, the clostridial neurotoxin sequence comprised in said clostridial neurotoxin may consist of a clostridial neurotoxin L-chain. In one embodiment, the clostridial neurotoxin sequence comprised in said clostridial neurotoxin may consist of a clostridial neurotoxin translocation domain. In one embodiment, the clostridial neurotoxin sequence comprised in said clostridial neurotoxin may consist of a clostridial neurotoxin receptor binding domain. In one embodiment, the clostridial neurotoxin sequence comprised in said clostridial neurotoxin may consist of a clostridial neurotoxin L-chain and a clostridial neurotoxin translocation domain.

Suitable clostridial neurotoxins comprising (or consisting of) a clostridial neurotoxin L-chain and translocation domain are described herein.

A clostridial neurotoxin comprising (or consisting of) a clostridial neurotoxin L-chain and translocation domain may comprise a polypeptide sequence having at least 70% sequence identity to any one of SEQ ID NOs: 4, 20, 28 or 36 or a fragment thereof. In one a clostridial neurotoxin comprising (or consisting of) a clostridial neurotoxin L-chain and translocation domain comprises a polypeptide sequence having at least 80%, 90%, 95% or 98% sequence identity to any one of SEQ ID NOs: 4, 20, 28 or 36 or a fragment thereof. Preferably, a clostridial neurotoxin comprising (or consisting of) a clostridial neurotoxin L-chain and translocation domain comprises (more preferably consists of) a polypeptide sequence comprising any one of SEQ ID NOs: 4, 20, 28 or 36 or a fragment thereof.

A clostridial neurotoxin comprising (or consisting of) a clostridial neurotoxin L-chain and translocation domain may be one encoded by a nucleotide sequence having at least 70% sequence identity to any one of SEQ ID NOs: 3, 19, 27 or 35 or a fragment thereof. In one embodiment a clostridial neurotoxin comprising (or consisting of) a clostridial neurotoxin L-chain and translocation domain is one encoded by a nucleotide sequence having at least 80%, 90%, 95% or 98% sequence identity to any one of SEQ ID NOs: 3, 19, 27 or 35 or a fragment thereof. Preferably, a clostridial neurotoxin comprising (or consisting of) a clostridial neurotoxin L-chain and translocation domain is one encoded by a nucleotide sequence comprising any one of SEQ ID NOs: 3, 19, 27 or 35 or a fragment thereof.

The clostridial neurotoxin of the present invention may be free from the complexing proteins that are present in a naturally occurring clostridial neurotoxin complex.

The clostridial neurotoxins of the present invention can be produced using recombinant nucleic acid technologies. Thus, in one embodiment, a clostridial neurotoxin (as described above) is a recombinant clostridial neurotoxin.

In one embodiment a clostridial neurotoxin of the invention comprises a clostridial neurotoxin L-chain. It is preferred that the L-chain is catalytically inactive.

Active clostridial neurotoxin L-chain has non-cytotoxic protease activity. Specifically, active clostridial neurotoxin L-chain has endopeptidase activity and is capable of cleaving a protein of the exocytic fusion apparatus in a target cell. A protein of the exocytic fusion apparatus is preferably a SNARE protein, such as SNAP-25, synaptobrevin/VAMP, or syntaxin.

The term “catalytically inactive” as used herein in respect of a clostridial neurotoxin L-chain means that said L-chain exhibits substantially no non-cytotoxic protease activity, preferably the term “catalytically inactive” as used herein in respect of a clostridial neurotoxin L-chain means that said L-chain exhibits no non-cytotoxic protease activity. In one embodiment, a catalytically inactive clostridial neurotoxin L-chain is one that does not cleave a protein of the exocytic fusion apparatus in a target cell. The term “substantially no non-cytotoxic protease activity” means that the clostridial neurotoxin L-chain has less than 5% of the non-cytotoxic protease activity of a catalytically active clostridial neurotoxin L-chain, for example less than 2%, 1% or preferably less than 0.1% of the non-cytotoxic protease activity of a catalytically active clostridial neurotoxin L-chain. Non-cytotoxic protease activity can be determined in vitro by incubating a test clostridial neurotoxin L-chain with a SNARE protein and comparing the amount of SNARE protein cleaved by the test clostridial neurotoxin L-chain when compared to the amount of SNARE protein cleaved by a catalytically active clostridial neurotoxin L-chain under the same conditions. Routine techniques, such as SDS-PAGE and Western blotting can be used to quantify the amount of SNARE protein cleaved. Suitable in vitro assays are described in WO 2019/145577 A1, which is incorporated herein by reference.

Cell-based and in vivo assays may also be used to determine if a clostridial neurotoxin comprising an L-chain and a functional cell binding and translocation domain has non-cytotoxic protease activity. Assays such as the Digit Abduction Score (DAS), the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Donald et al (2018), Pharmacol Res Perspect, e00446, 1-14, which is incorporated herein by reference.

A catalytically inactive L-chain may have one or more mutations that inactivate said catalytic activity. For example, a catalytically inactive BoNT/A L-chain may comprise a mutation of an active site residue, such as His223, Glu224, His227, Glu262, and/or Tyr366. The position numbering corresponds to the amino acid positions of SEQ ID NO: 62 and can be determined by aligning a polypeptide with SEQ ID NO: 62. As the presence of a methionine residue at position 1 of SEQ ID NO: 62 is optional, the skilled person will take the presence/absence of the methionine residue into account when determining amino acid residue numbering. For example, where SEQ ID NO: 62 includes a methionine, the position numbering will be as defined above (e.g. His223 will be His223 of SEQ ID NO: 62). Alternatively, where the methionine is absent from SEQ ID NO: 62 the amino acid residue numbering should be modified by −1 (e.g. His223 will be His222 of SEQ ID NO: 62). Similar considerations apply when the methionine at position 1 of the other polypeptide sequences described herein is present/absent, and the skilled person will readily determine the correct amino acid residue numbering using techniques routine in the art.

In a particularly preferred embodiment, a clostridial neurotoxin of the invention may comprise a modified BoNT/A or fragment thereof (preferably a BoNT/A H_{C} domain or fragment thereof). The modified BoNT/A or fragment thereof may be one that comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277. Such a modified BoNT/A or fragment thereof may demonstrate a reduction in, or absence of, side effects compared to the use of known BoNT/A. The increased tissue retention properties of the modified BoNT/A of the invention may also provide increased potency and/or duration of action and can allow for reduced dosages to be used compared to known clostridial toxin therapeutics (or increased dosages without any additional adverse effects), thus providing further advantages.

The modification may be a modification when compared to unmodified BoNT/A shown as SEQ ID NO: 62, wherein the amino acid residue numbering is determined by alignment with SEQ ID NO: 62. As the presence of a methionine residue at position 1 of SEQ ID NO: 62 (as well as the SEQ ID NOs corresponding to modified BoNT/A polypeptides or fragments thereof described herein) is optional, the skilled person will take the presence/absence of the methionine residue into account when determining amino acid residue numbering. For example, where SEQ ID NO: 62 includes a methionine, the position numbering will be as defined above (e.g. ASN 886 will be ASN 886 of SEQ ID NO: 62). Alternatively, where the methionine is absent from SEQ ID NO: 62 the amino acid residue numbering should be modified by −1 (e.g. ASN 886 will be ASN 885 of SEQ ID NO: 62). Similar considerations apply when the methionine at position 1 of the other polypeptide sequences described herein is present/absent, and the skilled person will readily determine the correct amino acid residue numbering using techniques routine in the art.

The amino acid residue(s) indicated for modification above are surface exposed amino acid residue(s).

A modified BoNT/A or fragment thereof may comprise a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277.

The term “one or more amino acid residue(s)” when used in the context of modified BoNT/A or fragment thereof preferably means at least 2, 3, 4, 5, 6 or 7 of the indicated amino acid residue(s). Thus, a modified BoNT/A may comprise at least 2, 3, 4, 5, 6 or 7 (preferably ≤7) modifications at the indicated amino acid residue(s). A modified BoNT/A or fragment thereof may comprise 1-30, 3-20, or 5-10 amino acid modifications. More preferably, the term “one or more amino acid residue(s)” when used in the context of modified BoNT/A or fragment thereof means all of the indicated amino acid residue(s).

Preferably, beyond the one or more amino acid modification(s) at the indicated amino acid residue(s), the modified BoNT/A or fragment thereof does not contain any further amino acid modifications when compared to SEQ ID NO: 62.

The modification may be selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

A modification as indicated above results in a modified BoNT/A or fragment thereof that has an increased positive surface charge and increased isoelectric point when compared to the corresponding unmodified BoNT/A or fragment thereof.

The isoelectric point (pI) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group). Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein. Accordingly, for every protein there is a given pH at which the number of positive and negative charges is equal and the protein displays no overall net charge. This point is known as the isoelectric point (pI). The isoelectric point is a standard concept in protein biochemistry with which the skilled person would be familiar.

The isoelectric point (pI) is therefore defined as the pH value at which a protein displays a net charge of zero. An increase in pI means that a higher pH value is required for the protein to display a net charge of zero. Thus, an increase in pI represents an increase in the net positive charge of a protein at a given pH. Conversely, a decrease in pI means that a lower pH value is required for the protein to display a net charge of zero. Thus, a decrease in pI represents a decrease in the net positive charge of a protein at a given pH.

Methods of determining the pI of a protein are known in the art and would be familiar to a skilled person. By way of example, the pI of a protein can be calculated from the average pKa values of each amino acid present in the protein (“calculated pI”). Such calculations can be performed using computer programs known in the art, such as the Compute pI/MW Tool from ExPASy (https://web.expasy.org/compute_pi/), which is the preferred method for calculating pI in accordance with the present invention. Comparisons of pI values between different molecules should be made using the same calculation technique/program.

Where appropriate, the calculated pI of a protein can be confirmed experimentally using the technique of isoelectric focusing (“observed pI”). This technique uses electrophoresis to separate proteins according to their pI. Isoelectric focusing is typically performed using a gel that has an immobilised pH gradient. When an electric field is applied, the protein migrates through the pH gradient until it reaches the pH at which it has zero net charge, this point being the pI of the protein. Results provided by isoelectric focusing are typically relatively low-resolution in nature, and thus the present inventors believe that results provided by calculated pI (as described above) are more appropriate to use.

Throughout the present specification, “pI” means “calculated pI” unless otherwise stated.

The pI of a protein may be increased or decreased by altering the number of basic and/or acidic groups displayed on its surface. This can be achieved by modifying one or more amino acids of the protein. For example, an increase in pI may be provided by reducing the number of acidic residues, or by increasing the number of basic residues.

A modified BoNT/A or fragment thereof of the invention may have a pI value that is at least 0.2, 0.4, 0.5 or 1 pI units higher than that of an unmodified BoNT/A (e.g. SEQ ID NO: 62) or fragment thereof. Preferably, a modified BoNT/A or fragment thereof may have a pI of at least 6.6, e.g. at least 6.8.

The properties of the 20 standard amino acids are indicated in the table below:

| Amino Acid | | | Side Chain |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence or fragment thereof, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A or fragment thereof. In an amino acid substitution, an amino acid residue that forms part of the BoNT/A polypeptide sequence or fragment thereof is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Preferably, a modified BoNT/A or fragment thereof for use in the invention comprises between 4 and 40 amino acid modifications located in the clostridial toxin H_{CN} domain. Said modified BoNT/A or fragment thereof preferably also has pI of at least 6.6. Said modified BoNT/A preferably comprises modifications of at least 4 amino acids selected from: ASN 886, ASN 930, ASN 954, SER 955, GLN 991, ASN 1025, ASN 1026, and ASN 1052, wherein said modification comprises substitution of the amino acids with a lysine residue or an arginine residue. For example, said modified BoNT/A or fragment thereof may comprise modifications of at least 5 amino acids selected from: ASN 886, ASN 930, ASN 954, SER 955, GLN 991, ASN 1025, ASN 1026, ASN 1052, and GLN 1229, wherein said modification comprises substitution of the amino acids with a lysine residue or an arginine residue.

Methods for modifying proteins by substitution, insertion or deletion of amino acid residues are known in the art. By way of example, amino acid modifications may be introduced by modification of a DNA sequence encoding a polypeptide (e.g. encoding unmodified BoNT/A or a fragment thereof). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised.

In one embodiment a clostridial neurotoxin for use according to the invention comprises a polypeptide sequence having at least 80%, 90%, 95% or 98% sequence identity to SEQ ID NO: 42. Preferably, a clostridial neurotoxin for use according to the invention comprises a polypeptide sequence shown as SEQ ID NO: 42.

In one embodiment a clostridial neurotoxin for use according to the invention comprises a polypeptide sequence that is encoded by a nucleotide sequence having at least 80%, 90%, 95% or 98% sequence identity to SEQ ID NO: 41. Preferably, a clostridial neurotoxin for use according to the invention comprises a polypeptide sequence that is encoded by a nucleotide sequence shown as SEQ ID NO: 41.

In one embodiment a clostridial neurotoxin for use according to the invention (e.g. comprising SEQ ID NO: 42 or encoded by SEQ ID NO: 41) may be a portion of a polypeptide having at least 70% sequence identity to SEQ ID NO: 61 or 65. Thus, in one embodiment a clostridial neurotoxin for use according to the invention may comprise a polypeptide sequence having at least 80%, 90%, 95% or 98% sequence identity to SEQ ID NO: 61 or 65. Preferably, a clostridial neurotoxin for use according to the invention may comprise (more preferably consist of) SEQ ID NO: 61 or 65. In one embodiment the clostridial neurotoxin comprises a catalytically-inactive L-chain (e.g. as per SEQ ID NO: 65).

In one embodiment a clostridial neurotoxin for use according to the invention (e.g. comprising SEQ ID NO: 42 or encoded by SEQ ID NO: 41) may be encoded by a nucleotide sequence having at least 70% sequence identity to SEQ ID NO: 60. Thus, in one embodiment a clostridial neurotoxin for use according to the invention may be encoded by a nucleotide sequence having at least 80%, 90%, 95% or 98% sequence identity to SEQ ID NO: 60. Preferably, a clostridial neurotoxin for use according to the invention may be encoded by a nucleotide sequence comprising (more preferably consisting of) SEQ ID NO: 60. In one embodiment the clostridial neurotoxin comprises a catalytically-inactive L-chain.

SEQ ID NO: 42 is an example of a modified BoNT/A fragment and SEQ ID NOs: 61 and 65 are examples of modified BoNT/A polypeptides that are catalytically active and inactive, respectively. Such modified BoNT/A polypeptides and fragments are particularly preferred for use in the present invention. The polypeptides shown as SEQ ID NO: 42, 61 and 62 have a number of amino acid modifications (e.g. substitutions) when compared to wild-type BoNT/A, which increase the isoelectric point of the polypeptide. Without wishing to be bound by theory, it is believed that the increased net positive charge promotes electrostatic interactions between the polypeptide and anionic extracellular components, thereby promoting binding between the polypeptide and cell surface thus increasing retention at a site of administration and/or duration of action. Thus, it is envisaged that neuroimmune response-promotion properties of SEQ ID NO: 42, 61 and 65 will be improved compared to equivalent polypeptides lacking said modifications.

For the catalytically active modified BoNT/A polypeptides described above (e.g. SEQ ID NO: 61), one way in which these advantageous properties (which represent an increase in the therapeutic index) may be defined is in terms of the Safety Ratio of the modified BoNT/A. In this regard, undesired effects of a clostridial neurotoxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial neurotoxin can be assessed experimentally by Digital Abduction Score (DAS) assay, a measurement of muscle paralysis. The DAS assay may be performed by injection of 20 μl of clostridial neurotoxin, formulated in Gelatin Phosphate Buffer, into the mouse gastrocnemius/soleus complex, followed by assessment of Digital Abduction Score using the method of Aoki (Aoki KR, Toxicon 39: 1815-1820; 2001). In the DAS assay, mice are suspended briefly by the tail in order to elicit a characteristic startle response in which the mouse extends its hind limbs and abducts its hind digits. Following clostridial neurotoxin injection, the varying degrees of digit abduction are scored on a five-point scale (0=normal to 4=maximal reduction in digit abduction and leg extension).

The Safety Ratio of a clostridial neurotoxin may then be expressed as the ratio between the amount of toxin required for a 10% drop in a bodyweight (measured at peak effect within the first seven days after dosing in a mouse) and the amount of toxin required for a DAS score of 2. High Safety Ratio scores are therefore desired and indicate a toxin that is able to effectively paralyse a target muscle with little undesired off-target effects. A catalytically active modified BoNT/A of the present invention may have a Safety Ratio that is higher than the Safety Ratio of an equivalent unmodified (native) botulinum toxin (e.g. SEQ ID NO: 62).

Thus, in one embodiment, a catalytically active modified BoNT/A of the present invention has a Safety Ratio of at least 8 (for example, at least 8, 9, 10, 15, 20, 25, 30, 35, 40, 45 or 50), wherein Safety Ratio is calculated as: dose of toxin required for −10% bodyweight change (pg/mouse) divided by DAS ED₅₀ (pg/mouse) [ED₅₀=dose required to produce a DAS score of 2].

In one embodiment, a catalytically active modified BoNT/A of the present invention has a Safety Ratio of at least 10. In one embodiment, a modified BoNT/A or fragment thereof of the present invention has a Safety Ratio of at least 15.

Clostridial neurotoxins comprising at least 70% sequence identity to SEQ ID NO: 61 are described in WO 2015/004461 A1, which is incorporated herein by reference in its entirety.

In one embodiment a clostridial neurotoxin comprising a polypeptide sequence having at least 70% sequence identity to SEQ ID NO: 42, 61 or 65 and/or comprising a polypeptide sequence that is encoded by a nucleotide sequence having at least 70% sequence identity to SEQ ID NO: 41 or 60 comprises a substitution at one or more (preferably two or more, three or more, four or more, five or more or six or more, more preferably at all) of positions 930, 955, 991, 1026, 1052, 1229, and 886. The position numbering corresponds to the positions of SEQ ID NO: 62 and can be determined by aligning the polypeptide sequence with SEQ ID NO: 62 (unmodified/wild-type BoNT/A). As the presence of a methionine residue at position 1 of SEQ ID NO: 62 is optional, the skilled person will take the presence/absence of the methionine residue into account when determining amino acid residue numbering. For example, where SEQ ID NO: 62 includes a methionine, the position numbering will be as defined above (e.g. position 886 will be ASN 886 of SEQ ID NO: 62). Alternatively, where the methionine is absent from SEQ ID NO: 62 the amino acid residue numbering should be modified by −1 (e.g. position 886 will be ASN 885 of SEQ ID NO: 62). Similar considerations apply when the methionine at position 1 of the other polypeptide sequences described herein is present/absent, and the skilled person will readily determine the correct amino acid residue numbering using techniques routine in the art.

Preferably, the clostridial neurotoxin comprising a polypeptide sequence having at least 70% sequence identity to SEQ ID NO: 42, 61 or 65 and/or comprising a polypeptide sequence that is encoded by a nucleotide sequence having at least 70% sequence identity to SEQ ID NO: 41 or 60 comprises lysine or arginine (more preferably lysine) at one or more of positions 930, 955, 991, 1026, 1052, 1229, and 886. In one embodiment, the clostridial neurotoxin comprises lysine or arginine (more preferably lysine) at least two, three, four, five, six or all of positions 930, 955, 991, 1026, 1052, 1229, and 886. Most preferably, the clostridial neurotoxin comprises lysine or arginine (more preferably lysine) at all of positions 930, 955, 991, 1026, 1052, 1229, and 886.

Embodiments related to the various therapeutic uses of the invention are intended to be applied equally to methods of treatment, polypeptides of the invention, and vice versa.

Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

Alignment Scores for Determining Sequence Identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

Conservative Amino Acid Substitutions
Basic: arginine, lysine, histidine
Acidic: glutamic acid, aspartic acid
Polar: glutamine, asparagine
Hydrophobic: leucine, isoleucine, valine
Aromatic: phenylalanine, tryptophan, tyrosine
Small: glycine, alanine, serine, threonine, methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethyl homo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WI PO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WI PO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such agents and reference to “the clostridial neurotoxin” includes reference to one or more clostridial neurotoxin and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1. Schematic diagram of imaging areas' location in the study.

FIG. 2. Graph showing laser injury-induced changes in microglia density in Area 1. In Dysport-treated mice, more microglia migrated towards the lesion site (increasing microglia cell density).

FIG. 3. Graph showing laser-induced activation changes in microglia mean sphericity index in Area 1. Activated microglia have a lower sphericity index (cell soma shape is more elongated).

FIG. 4. Graph showing fluorescence change after laser-induced microglia process extension (outliers removed; scar formation in Area 1): microglia response was significantly stronger at 42 minutes in Dysport-treated mice.

### SEQUENCE LISTING

Where an initial Met amino acid residue or a corresponding initial codon is indicated in any of the following SEQ ID NOs, said residue/codon is optional.
SEQ ID NO: 1—Nucleotide Sequence of Recombinant Catalytically Inactive BoNT/A (rBoNT/A(0))
SEQ ID NO: 2—Polypeptide Sequence of rBoNT/A(0)
SEQ ID NO: 3—Nucleotide Sequence of rLH_{N}/A (light-chain plus translocation domain only).
SEQ ID NO: 4—Polypeptide Sequence of rLH_{N}/A
SEQ ID NO: 5—Nucleotide Sequence of rL/A (light-chain only)
SEQ ID NO: 6—Polypeptide Sequence of rL/A
SEQ ID NO: 7—Nucleotide Sequence of rH_{C}/A
SEQ ID NO: 8—Polypeptide Sequence of rH_{C}/A
SEQ ID NO: 9—Nucleotide Sequence of rBoNT/B(0)
SEQ ID NO: 10—Polypeptide Sequence of rBoNT/B(0)
SEQ ID NO: 11—Nucleotide Sequence of rBoNT/C(0)
SEQ ID NO: 12—Polypeptide Sequence of rBoNT/C(0)
SEQ ID NO: 13—Nucleotide Sequence of rBoNT/E(0)
SEQ ID NO: 14—Polypeptide Sequence of rBoNT/E(0)
SEQ ID NO: 15—Nucleotide Sequence of rBoNT/F(0)
SEQ ID NO: 16—Polypeptide Sequence of rBoNT/F(0)
SEQ ID NO: 17—Nucleotide Sequence of rBoNT/A(0) (His-tagged)
SEQ ID NO: 18—Polypeptide Sequence of rBoNT/A(0) (His-tagged)
SEQ ID NO: 19—Nucleotide Sequence of rLH_{N}/A (His-tagged)
SEQ ID NO: 20—Polypeptide Sequence of rLH_{N}/A (His-tagged)
SEQ ID NO: 21—Nucleotide Sequence of rH_{C}/A (His-tagged)
SEQ ID NO: 22—Polypeptide Sequence of rH_{C}/A (His-tagged)
SEQ ID NO: 23—Nucleotide Sequence of rLC/A (His-tagged)
SEQ ID NO: 24—Polypeptide Sequence of rLC/A (His-tagged)
SEQ ID NO: 25—Nucleotide Sequence of rBoNT/FA(0) (His-tagged)
SEQ ID NO: 26—Polypeptide Sequence of rBoNT/FA(0) (His-tagged)
SEQ ID NO: 27—Nucleotide Sequence of rLH_{N}/FA (His-tagged)
SEQ ID NO: 28—Polypeptide Sequence of rLH_{N}/FA (His-tagged)
SEQ ID NO: 29—Nucleotide Sequence of rH_{C}/FA (His-tagged)
SEQ ID NO: 30—Polypeptide Sequence of rH_{C}/FA (His-tagged)
SEQ ID NO: 31—Nucleotide Sequence of rLC/FA (His-tagged)
SEQ ID NO: 32—Polypeptide Sequence of rLC/FA (His-tagged)
SEQ ID NO: 33—Nucleotide Sequence of rBoNT/F(0) (His-tagged)
SEQ ID NO: 34—Polypeptide Sequence of rBoNT/F(0) (His-tagged)
SEQ ID NO: 35—Nucleotide Sequence of rL H_{N}/F (His-tagged)
SEQ ID NO: 36—Polypeptide Sequence of rL H_{N}/F (His-tagged)
SEQ ID NO: 37—Nucleotide Sequence of rH_{C}/F (His-tagged)
SEQ ID NO: 38—Polypeptide Sequence of rH_{C}/F (His-tagged)
SEQ ID NO: 39—Nucleotide Sequence of rLC/F (His-tagged)
SEQ ID NO: 40—Polypeptide Sequence of rLC/F (His-tagged)
SEQ ID NO: 41—Nucleotide Sequence of Cationic rH_{C}/A (His-tagged)
SEQ ID NO: 42—Polypeptide Sequence of Cationic rH_{C}/A (His-tagged)
SEQ ID NO: 43—Nucleotide Sequence of rH_{C}/AB (His-tagged)
SEQ ID NO: 44—Polypeptide Sequence of rH_{C}/AB (His-tagged)
SEQ ID NO: 45—Nucleotide Sequence of rH_{C}/A Variant Y1117V H1253K (His-tagged)
SEQ ID NO: 46—Polypeptide Sequence of rH_{C}/A Variant Y1117V H1253K (His-tagged)
SEQ ID NO: 47—Nucleotide Sequence of rH_{C}/A Variant Y1117V F1252Y H1253K L1278F (His-tagged)
SEQ ID NO: 48—Polypeptide Sequence of rH_{C}/A Variant Y1117V F1252Y H1253K L1278F (His-tagged)
SEQ ID NO: 49—Nucleotide Sequence of rH_{C}/A Variant Y1117V F1252Y H1253K L1278H (His-tagged)
SEQ ID NO: 50—Polypeptide Sequence of rH_{C}/A Variant Y1117V F1252Y H1253K L1278H (His-tagged)
SEQ ID NO: 51—Polypeptide Sequence of BoNT/A—UniProt P10845
SEQ ID NO: 52—Polypeptide Sequence of BoNT/B—UniProt P10844
SEQ ID NO: 53—Polypeptide Sequence of BoNT/C—UniProt P18640
SEQ ID NO: 54—Polypeptide Sequence of BoNT/D—UniProt P19321
SEQ ID NO: 55—Polypeptide Sequence of BoNT/E—UniProt Q00496
SEQ ID NO: 56—Polypeptide Sequence of BoNT/F—UniProt A7GBG3
SEQ ID NO: 57—Polypeptide Sequence of BoNT/G—UniProt Q60393
SEQ ID NO: 58—Polypeptide Sequence of TeNT—UniProt P04958
SEQ ID NO: 59—Polypeptide Sequence of BoNT/X
SEQ ID NO: 60—Nucleotide Sequence of mrBoNT/A
SEQ ID NO: 61—Polypeptide Sequence of mrBoNT/A
SEQ ID NO: 62—Polypeptide Sequence of Unmodified BoNT/A1
SEQ ID NO: 63—Polypeptide Sequence of mrBoNT/AB
SEQ ID NO: 64—Polypeptide Sequence of mrBoNT/AB(0)
SEQ ID NO: 65—Polypeptide Sequence of mrBoNT/A(0)

Nucleotide Sequence of rBoNT/A(0)
SEQ ID NO: 1
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATCAACTG
ATCtACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATTGATCTGAGCCGTTATGCAAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAACGCCATTGTCTACAACTCCATGTACGAGAATTTCTCCACCAGCTTCTGGATTCGCATCCCG
AAATACTTCAACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AACAGCAAGATTTACATCAATGGTCGCTTGATCGATCAGAAACCGATTAGCAACCTGGGTAATATCCACGCAAGC
AACAACATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACCAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
Polypeptide Sequence of rBoNT/A(0)
SEQ ID NO: 2
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYENSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
Nucleotide Sequence of rLH_{N}/A
SEQ ID NO: 3
atggagttcgttaacaaacagttcaactataaagacccagttaacggtgttgacattgcttacatcaaaatcccg
aacgctggccagatgcagccggtaaaggcattcaaaatccacaacaaaatctgggttatcccggaacgtgatacc
tttactaacccggaagaaggtgacctgaacccgccaccggaagcgaaacaggtgccggtatcttactatgactcc
acctacctgtctaccgataacgaaaaggacaactacctgaaaggtgttactaaactgttcgagcgtatttactcc
accgacctgggccgtatgctgctgactagcatcgttcgcggtatcccgttctggggcggttctaccatcgatacc
gaactgaaagtaatcgacactaactgcatcaacgttattcagccggacggttcctatcgttccgaagaactgaac
ctggtgatcatcggcccgtctgctgatatcatccagttcgagtgtaagagctttggtcacgaagttctgaacctc
acccgtaacggctacggttccactcagtacatccgtttctctccggacttcaccttcggttttgaagaatccctg
gaagtagacacgaacccactgctgggcgctggtaaattcgcaactgatcctgcggttaccctggctcacgaactg
attcatgcaggccaccgcctgtacggtatcgccatcaatccgaaccgtgtcttcaaagttaacaccaacgcgtat
tacgagatgtccggtctggaagttagcttcgaagaactgcgtacttttggcggtcacgacgctaaattcatcgac
tctctgcaagaaaacgagttccgtctgtactactataacaagttcaaagatatcgcatccaccctgaacaaagcg
aaatccatcgtgggtaccactgcttctctccagtacatgaagaacgtttttaaagaaaaatacctgctcagcgaa
gacacctccggcaaattctctgtagacaagttgaaattcgataaactttacaaaatgctgactgaaatttacacc
gaagacaacttcgttaagttctttaaagttctgaaccgcaaaacctatctgaacttcgacaaggcagtattcaaa
atcaacatcgtgccgaaagttaactacactatctacgatggtttcaacctgcgtaacaccaacctggctgctaat
tttaacggccagaacacggaaatcaacaacatgaacttcacaaaactgaaaaacttcactggtctgttcgagttt
tacaagctgctgtgcGTCGACGGCATCATTACCTCCAAAACTAAATCTGACGATGACGATAAAAACAAAGCGCTG
AACCTGCAGtgtatcaaggttaacaactgggatttattcttcagcccgagtgaagacaacttcaccaacgacctg
aacaaaggtgaagaaatcacctcagatactaacatcgaagcagccgaagaaaacatctcgctggacctgatccag
cagtactacctgacctttaatttcgacaacgagccggaaaacatttctatcgaaaacctgagctctgatatcatc
ggccagctggaactgatgccgaacatcgaacgtttcccaaacggtaaaaagtacgagctggacaaatataccatg
ttccactacctgcgcgcgcaggaatttgaacacggcaaatcccgtatcgcactgactaactccgttaacgaagct
ctgctcaacccgtcccgtgtatacaccttcttctctagcgactacgtgaaaaaggtcaacaaagcgactgaagct
gcaatgttcttgggttgggttgaacagcttgtttatgattttaccgacgagacgtccgaagtatctactaccgac
aaaattgcggatatcactatcatcatcccgtacatcggtccggctctgaacattggcaacatgctgtacaaagac
gacttcgttggcgcactgatcttctccggtgcggtgatcctgctggagttcatcccggaaatcgccatcccggta
ctgggcacctttgctctggtttcttacattgcaaacaaggttctgactgtacaaaccatcgacaacgcgctgagc
aaacgtaacgaaaaatgggatgaagtttacaaatatatcgtgaccaactggctggctaaggttaatactcagatc
gacctcatccgcaaaaaaatgaaagaagcactggaaaaccaggcggaagctaccaaggcaatcattaactaccag
tacaaccagtacaccgaggaagaaaaaaacaacatcaacttcaacatcgacgatctgtcctctaaactgaacgaa
tccatcaacaaagctatgatcaacatcaacaagttcctgaaccagtgctctgtaagctatctgatgaactccatg
atcccgtacggtgttaaacgtctggaggacttcgatgcgtctctgaaagacgccctgctgaaatacatttacgac
aaccgtggcactctgatcggtcaggttgatcgtctgaaggacaaagtgaacaataccttatcgaccgacatccct
tttcagctcagtaaatatgtcgataaccaacgccttttgtccactctagaagcaCACCATCATCACcaccatcac
catcaccat
Polypeptide Sequence of rLH_{N}/A
SEQ ID NO: 4
MEFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVDGIITSKTKSDDDDKNKAL
NLQCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTLEAHHHHHHHHHH
Nucleotide Sequence of rL/A
SEQ ID NO: 5
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGggtctagaagcaCACCATCATCACcaccatcaccatcaccat
Polypeptide Sequence of rL/A
SEQ ID NO: 6
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLGLEAHHHHHHHHHH
Nucleotide Sequence of rH_{C}/A
SEQ ID NO: 7
ATGCATCATCACCATCACCACAAAAACATCATCAATACTAGCATTCTGAACCTGCGTTACGAGAGCAATCATCTG
ATTGATCTGAGCCGTTATGCAAGCAAGATCAACATCGGTAGCAAGGTCAATTTTGACCCGATCGATAAGAACCAG
ATCCAGCTGTTTAATCTGGAATCGAGCAAAATTGAGGTTATCCTGAAAAACGCCATTGTCTACAACTCCATGTAC
GAGAATTTCTCCACCAGCTTCTGGATTCGCATCCCGAAATACTTCAACAGCATTAGCCTGAACAACGAGTATACT
ATCATCAACTGTATGGAGAACAACAGCGGTTGGAAGGTGTCTCTGAACTATGGTGAGATCATTTGGACCTTGCAG
GACACCCAAGAGATCAAGCAGCGCGTCGTGTTCAAGTACTCTCAAATGATCAACATTTCCGATTACATTAATCGT
TGGATCTTCGTGACCATTACGAATAACCGTCTGAATAACAGCAAGATTTACATCAATGGTCGCTTGATCGATCAG
AAACCGATTAGCAACCTGGGTAATATCCACGCAAGCAACAACATTATGTTCAAATTGGACGGTTGCCGCGATACC
CATCGTTATATCTGGATCAAGTATTTCAACCTGTTTGATAAAGAACTGAATGAGAAGGAGATCAAAGATTTGTAT
GACAACCAATCTAACAGCGGCATTTTGAAGGACTTCTGGGGCGATTATCTGCAATACGATAAGCCGTACTATATG
CTGAACCTGTATGATCCGAACAAATATGTGGATGTCAATAATGTGGGTATTCGTGGTTACATGTATTTGAAGGGT
CCGCGTGGCAGCGTTATGACGACCAACATTTACCTGAACTCTAGCCTGTACCGTGGTACGAAATTCATCATTAAG
AAATATGCCAGCGGCAACAAAGATAACATTGTGCGTAATAACGATCGTGTCTACATCAACGTGGTCGTGAAGAAT
AAAGAGTACCGTCTGGCGACCAACGCTTCGCAGGCGGGTGTTGAGAAAATTCTGAGCGCGTTGGAGATCCCTGAT
GTCGGTAATCTGAGCCAAGTCGTGGTTATGAAGAGCAAGAACGACCAGGGTATCACTAACAAGTGCAAGATGAAC
CTGCAAGACAACAATGGTAACGACATCGGCTTTATTGGTTTCCACCAGTTCAACAATATTGCTAAACTGGTAGCG
AGCAATTGGTACAATCGTCAGATTGAGCGCAGCAGCCGTACTTTGGGCTGTAGCTGGGAGTTTATCCCGGTCGAT
GATGGTTGGGGCGAACGTCCGCTG
Polypeptide Sequence of rH_{C}/A
SEQ ID NO: 8
MHHHHHHKNIINTSILNLRYESNHLIDLSRYASKINIGSKVNFDPIDKNQIQLENLESSKIEVILKNAIVYNSMY
ENFSTSFWIRIPKYENSISLNNEYTIINCMENNSGWKVSLNYGEIIWTLQDTQEIKQRVVFKYSQMINISDYINR
WIFVTITNNRLNNSKIYINGRLIDQKPISNLGNIHASNNIMFKLDGCRDTHRYIWIKYFNLEDKELNEKEIKDLY
DNQSNSGILKDFWGDYLQYDKPYYMLNLYDPNKYVDVNNVGIRGYMYLKGPRGSVMTTNIYLNSSLYRGTKFIIK
KYASGNKDNIVRNNDRVYINVVVKNKEYRLATNASQAGVEKILSALEIPDVGNLSQVVVMKSKNDQGITNKCKMN
LQDNNGNDIGFIGFHQFNNIAKLVASNWYNRQIERSSRTLGCSWEFIPVDDGWGERPL
Nucleotide Sequence of rBoNT/B(0)
SEQ ID NO: 9
ATGCCGGTGACGATTAACAACTTCAACTACAACGACCCGATTGACAACAACAACATTATCATGATGGAACCGCCG
TTTGCACGCGGCACGGGCCGTTATTACAAAGCGTTTAAAATCACCGATCGTATTTGGATTATCCCGGAACGCTAC
ACGTTTGGTTATAAACCGGAAGACTTCAACAAAAGCTCTGGCATCTTCAACCGTGATGTTTGCGAATACTACGAT
CCGGACTACCTGAACACCAACGATAAGAAAAACATTTTTCTGCAAACGATGATCAAACTGTTCAATCGCATTAAA
AGCAAACCGCTGGGTGAAAAACTGCTGGAAATGATTATCAATGGCATTCCGTATCTGGGTGATCGTCGCGTGCCG
CTGGAAGAATTTAACACCAATATCGCGAGTGTTACGGTCAACAAACTGATTTCCAATCCGGGTGAAGTCGAACGT
AAAAAAGGCATCTTCGCCAACCTGATCATCTTCGGCCCGGGTCCGGTGCTGAACGAAAATGAAACCATTGATATC
GGTATTCAGAACCATTTTGCCTCACGCGAAGGCTTCGGCGGTATTATGCAAATGAAATTTTGCCCGGAATATGTG
TCGGTTTTCAACAATGTTCAGGAAAACAAAGGTGCAAGCATCTTTAATCGTCGCGGCTATTTCTCTGATCCGGCT
CTGATCCTGATGCACCAACTGATTtATGTGCTGCACGGCCTGTATGGTATCAAAGTGGATGACCTGCCGATCGTT
CCGAACGAGAAAAAATTTTTCATGCAGAGCACCGACGCAATTCAAGCTGAAGAACTGTATACGTTTGGCGGTCAG
GACCCGTCTATTATCACCCCGAGCACCGACAAAAGCATCTACGATAAAGTGCTGCAAAACTTTCGTGGCATTGTT
GACCGCCTGAATAAAGTCCTGGTGTGTATCTCTGATCCGAACATCAACATCAACATCTACAAAAACAAATTCAAA
GACAAATACAAATTCGTTGAAGATTCTGAAGGCAAATATAGTATTGACGTCGAATCCTTTGATAAACTGTACAAA
AGTCTGATGTTCGGTTTCACCGAAACGAACATCGCGGAAAACTACAAAATCAAAACCCGCGCCTCCTATTTCAGC
GACTCTCTGCCGCCGGTTAAAATCAAAAATCTGCTGGATAACGAAATTTATACGATCGAAGAAGGTTTCAACATC
AGCGATAAAGACATGGAAAAAGAATACCGTGGCCAGAATAAAGCAATCAACAAACAGGCGTATGAAGAAATTAGT
AAAGAACATCTGGCGGTCTACAAAATTCAGATGTGCAAATCCGTGAAAGCCCCGGGTATTTGTATCGATGTTGAC
AATGAAGACCTGTTTTTCATCGCCGATAAAAACAGTTTTTCCGATGACCTGTCAAAAAATGAACGCATCGAATAC
AACACCCAATCGAACTACATCGAAAACGATTTCCCGATCAACGAACTGATTCTGGATACGGACCTGATTAGTAAA
ATCGAACTGCCGTCAGAAAACACCGAATCGCTGACGGACTTTAATGTTGATGTCCCGGTGTATGAAAAACAGCCG
GCAATTAAGAAAATTTTTACCGATGAAAACACGATCTTCCAGTACCTGTACAGCCAAACCTTTCCGCTGGACATT
CGCGATATCTCTCTGACGAGTTCCTTTGATGACGCACTGCTGTTCAGCAACAAAGTGTACTCCTTTTTCTCAATG
GATTACATCAAAACCGCTAACAAAGTGGTTGAAGCGGGCCTGTTTGCCGGTTGGGTGAAACAGATCGTTAACGAT
TTCGTCATCGAAGCCAACAAAAGTAACACGATGGATAAAATTGCTGATATCTCCCTGATTGTCCCGTATATTGGC
CTGGCACTGAATGTGGGTAACGAAACGGCGAAAGGCAATTTTGAAAACGCCTTCGAAATTGCAGGCGCTTCAATC
CTGCTGGAATTTATTCCGGAACTGCTGATCCCGGTCGTGGGTGCGTTCCTGCTGGAATCTTACATCGACAACAAA
AACAAAATCATCAAAACCATTGATAACGCGCTGACGAAACGTAACGAAAAATGGTCAGATATGTACGGCCTGATT
GTTGCCCAGTGGCTGAGCACCGTCAACACGCAATTTTACACCATCAAAGAAGGTATGTACAAAGCGCTGAATTAT
CAGGCGCAAGCCCTGGAAGAAATCATCAAATACCGCTACAACATCTACAGCGAAAAAGAAAAATCTAACATCAAC
ATCGACTTTAATGATATCAACAGCAAACTGAACGAAGGTATCAACCAGGCAATCGATAACATCAACAACTTCATC
AACGGCTGCTCAGTGTCGTATCTGATGAAGAAAATGATCCCGCTGGCTGTTGAAAAACTGCTGGATTTTGACAAC
ACCCTGAAGAAAAACCTGCTGAACTACATCGATGAAAACAAACTGTACCTGATCGGCTCAGCCGAATACGAAAAA
TCGAAAGTGAACAAATACCTGAAAACCATCATGCCGTTTGACCTGAGTATTTACACCAACGATACGATCCTGATC
GAAATGTTCAACAAATACAACTCCGAAATTCTGAACAATATTATCCTGAACCTGCGTTACAAAGACAACAATCTG
ATCGATCTGAGCGGCTATGGTGCAAAAGTTGAAGTCTACGACGGTGTCGAACTGAACGATAAAAACCAGTTCAAA
CTGACCTCATCGGCTAACTCAAAAATTCGTGTGACGCAGAACCAAAACATCATCTTCAACTCGGTCTTTCTGGAC
TTCAGCGTGTCTTTCTGGATTCGCATCCCGAAATATAAAAATGATGGCATCCAGAACTACATCCATAACGAATAC
ACCATCATCAACTGTATGAAAAACAACAGTGGTTGGAAAATTTCCATCCGTGGCAACCGCATTATCTGGACCCTG
ATTGATATCAATGGTAAAACGAAAAGCGTGTTTTTCGAATACAACATCCGTGAAGATATCTCTGAATACATCAAT
CGCTGGTTTTTCGTGACCATTACGAACAATCTGAACAATGCGAAAATCTATATCAACGGCAAACTGGAAAGTAAT
ACCGACATCAAAGATATTCGTGAAGTTATCGCCAACGGTGAAATCATCTTCAAACTGGATGGCGACATCGATCGC
ACCCAGTTCATTTGGATGAAATACTTCTCCATCTTCAACACGGAACTGAGTCAGTCCAATATCGAAGAACGCTAC
AAAATCCAATCATACTCGGAATACCTGAAAGATTTCTGGGGTAACCCGCTGATGTACAACAAAGAATACTACATG
TTCAACGCGGGCAACAAAAACTCATACATCAAACTGAAAAAAGATTCGCCGGTGGGTGAAATCCTGACCCGTAGC
AAATACAACCAGAACTCTAAATACATCAACTATCGCGATCTGTACATTGGCGAAAAATTTATTATCCGTCGCAAA
AGCAACTCTCAGAGTATTAATGATGACATCGTGCGTAAAGAAGACTACATCTATCTGGATTTCTTTAATCTGAAC
CAAGAATGGCGCGTTTATACCTACAAATACTTCAAAAAAGAAGAAGAGAAACTGTTCCTGGCCCCGATTAGCGAC
AGCGATGAATTTTACAACACCATCCAGATCAAAGAATACGATGAACAGCCGACGTATAGTTGCCAACTGCTGTTC
AAAAAAGACGAAGAATCCACCGATGAAATTGGCCTGATTGGTATCCACCGTTTCTATGAAAGCGGTATCGTTTTC
GAAGAATACAAAGATTACTTCTGTATCTCTAAATGGTATCTGAAAGAAGTCAAACGCAAACCGTACAACCTGAAA
CTGGGCTGCAACTGGCAATTTATCCCGAAAGACGAAGGCTGGACCGAA
Polypeptide Sequence of rBoNT/B(0)
SEQ ID NO: 10
MPVTINNENYNDPIDNNNIIMMEPPFARGTGRYYKAFKITDRIWIIPERYTFGYKPEDENKSSGIFNRDVCEYYD
PDYLNTNDKKNIFLQTMIKLFNRIKSKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYVSVENNVQENKGASIFNRRGYFSDPA
LILMHQLIYVLHGLYGIKVDDLPIVPNEKKFFMQSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGFTETNIAENYKIKTRASYFS
DSLPPVKIKNLLDNEIYTIEEGFNISDKDMEKEYRGQNKAINKQAYEEISKEHLAVYKIQMCKSVKAPGICIDVD
NEDLFFIADKNSESDDLSKNERIEYNTQSNYIENDFPINELILDTDLISKIELPSENTESLTDFNVDVPVYEKQP
AIKKIFTDENTIFQYLYSQTFPLDIRDISLTSSFDDALLESNKVYSFFSMDYIKTANKVVEAGLFAGWVKQIVND
FVIEANKSNTMDKIADISLIVPYIGLALNVGNETAKGNFENAFEIAGASILLEFIPELLIPVVGAFLLESYIDNK
NKIIKTIDNALTKRNEKWSDMYGLIVAQWLSTVNTQFYTIKEGMYKALNYQAQALEEIIKYRYNIYSEKEKSNIN
IDENDINSKLNEGINQAIDNINNFINGCSVSYLMKKMIPLAVEKLLDFDNTLKKNLLNYIDENKLYLIGSAEYEK
SKVNKYLKTIMPFDLSIYTNDTILIEMFNKYNSEILNNIILNLRYKDNNLIDLSGYGAKVEVYDGVELNDKNQFK
LTSSANSKIRVTQNQNIIFNSVFLDESVSFWIRIPKYKNDGIQNYIHNEYTIINCMKNNSGWKISIRGNRIIWTL
IDINGKTKSVFFEYNIREDISEYINRWFFVTITNNLNNAKIYINGKLESNTDIKDIREVIANGEIIFKLDGDIDR
TQFIWMKYFSIFNTELSQSNIEERYKIQSYSEYLKDFWGNPLMYNKEYYMFNAGNKNSYIKLKKDSPVGEILTRS
KYNQNSKYINYRDLYIGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCISKWYLKEVKRKPYNLK
LGCNWQFIPKDEGWTE
Nucleotide Sequence of rBoNT/C(0)
SEQ ID NO: 11
ATGCCGATCACGATTAATAATTTCAACTATAGCGATCCGGTGGACAATAAGAATATTCTGTATCTGGATACTCAT
CTGAATACGCTGGCTAACGAACCGGAGAAAGCGTTCCGCATCACAGGCAACATCTGGGTTATTCCCGATCGCTTT
TCACGCAACAGCAACCCTAATCTGAACAAACCTCCTCGTGTCACCAGTCCTAAATCCGGTTATTACGACCCAAAC
TATCTGAGTACGGATAGCGATAAAGATCCCTTTCTGAAAGAGATCATTAAGCTGTTCAAACGCATTAACTCTCGC
GAAATTGGGGAAGAGCTGATCTATCGGCTTTCGACAGATATCCCGTTCCCAGGTAACAATAATACCCCGATTAAT
ACTTTCGACTTTGATGTTGATTTCAATTCTGTGGATGTGAAAACGCGTCAAGGCAATAATTGGGTGAAAACTGGT
AGCATTAACCCGAGTGTAATTATCACAGGTCCCCGTGAGAACATCATCGACCCGGAAACCTCTACCTTCAAGCTG
ACGAACAACACGTTTGCTGCACAGGAAGGGTTTGGTGCCCTGTCAATCATTTCCATCTCACCGCGTTTCATGTTA
ACCTACTCCAATGCCACAAATGATGTTGGCGAAGGACGTTTTAGCAAATCAGAATTTTGCATGGACCCAATTCTC
ATTCTGATGggCacGCTGAACaATGCGATGCACAACTTGTATGGCATTGCTATTCCAAACGATCAAACCATTAGC
TCCGTTACCAGTAATATCTTCTATAGCCAGTATAATGTCAAATTGGAGTATGCCGAAATTTACGCCTTTGGAGGC
CCGACCATTGACCTGATTCCGAAATCTGCACGCAAATACTTCGAAGAAAAGGCGTTAGATTACTATCGCAGCATC
GCGAAACGCCTGAACTCGATTACCACGGCCAATCCGTCGTCGTTCAACAAATACATTGGTGAATATAAACAGAAA
CTGATTCGCAAATATCGGTTTGTCGTAGAAAGCTCTGGTGAAGTGACTGTAAACCGCAACAAATTTGTCGAACTC
TACAACGAGTTGACCCAAATCTTTACCGAGTTTAACTACGCAAAGATCTATAACGTACAGAACCGCAAGATTTAT
CTTAGCAATGTATACACACCGGTTACTGCGAACATCTTAGACGACAATGTGTATGATATTCAGAATGGCTTTAAC
ATCCCGAAATCAAATCTGAACGTTCTGTTTATGGGCCAGAACCTGAGTCGTAATCCAGCACTGCGTAAAGTGAAC
CCGGAAAATATGCTCTACTTGTTTACCAAATTTTGCCACAAAGCGATTGATGGCCGCTCTCTCTATAACAAAACG
CTGGATTGTCGTGAGTTACTTGTGAAGAACACTGATTTACCGTTCATTGGGGATATCTCCGACGTGAAAACCGAT
ATCTTCCTGCGCAAAGACATTAATGAAGAAACGGAAGTCATCTATTACCCCGACAATGTGAGCGTTGATCAGGTC
ATTTTATCGAAGAACACCTCCGAACATGGTCAGTTGGATTTGCTGTACCCTAGCATTGACTCGGAGAGTGAAATC
CTTCCGGGCGAAAATCAAGTGTTTTACGACAACCGTACCCAAAATGTTGATTATTTGAATTCTTATTACTACCTG
GAATCTCAGAAATTGAGCGACAATGTGGAAGATTTCACGTTCACACGCTCCATTGAGGAAGCGCTGGATAATAGC
GCGAAAGTGTATACGTATTTCCCTACCTTGGCGAATAAAGTAAATGCTGGTGTCCAGGGAGGCTTATTTCTGATG
TGGGCGAATGATGTGGTAGAAGATTTTACGACCAATATTTTGCGTAAGGACACCTTAGATAAAATTAGCGATGTT
AGCGCCATCATCCCCTATATTGGCCCAGCACTGAATATCTCGAACTCTGTGCGTCGCGGAAACTTCACCGAAGCA
TTTGCGGTGACCGGGGTTACTATTCTGTTGGAAGCCTTTCCGGAGTTTACTATTCCGGCGCTGGGTGCGTTTGTG
ATTTATTCGAAAGTACAAGAACGCAATGAAATTATCAAAACCATCGATAATTGCCTGGAACAACGCATTAAACGC
TGGAAGGATTCTTATGAATGGATGATGGGCACCTGGTTATCCCGTATTATCACACAGTTTAACAACATCTCGTAT
CAGATGTACGATTCACTGAACTACCAAGCAGGGGCGATCAAAGCCAAGATCGACTTAGAATACAAGAAATATTCA
GGTAGCGATAAAGAGAATATTAAAAGCCAGGTTGAAAACCTGAAGAACTCTCTGGATGTCAAAATTTCAGAGGCT
ATGAACAACATTAACAAATTTATCCGCGAATGTAGCGTCACGTATCTGTTTAAAAACATGCTCCCGAAAGTGATT
GATGAGCTCAACGAGTTTGATCGCAACACAAAGGCCAAACTGATTAACCTGATTGATAGTCACAATATTATTTTA
GTCGGTGAAGTTGACAAGCTGAAGGCTAAGGTCAATAACAGCTTTCAGAACACTATTCCGTTTAATATTTTCTCC
TATACGAACAATAGTCTGCTGAAAGACATTATCAACGAATACTTCAACAATATTAATGACAGCAAAATTCTGAGC
CTGCAGAATCGTAAGAATACGCTGGTAGATACCAGTGGATATAATGCGGAAGTCTCAGAAGAGGGTGATGTACAG
CTGAACCCGATCTTTCCGTTCGACTTTAAACTGGGGTCTAGTGGTGAAGATCGCGGTAAAGTGATCGTTACCCAA
AACGAGAACATTGTGTATAACAGCATGTACGAGAGTTTCTCAATTTCTTTCTGGATTCGCATCAATAAATGGGTT
TCTAATTTGCCTGGCTATACCATCATTGATAGCGTCAAAAACAACTCGGGCTGGTCGATTGGCATTATTAGCAAC
TTTCTGGTGTTTACCCTGAAACAGAATGAGGATTCGGAACAGAGCATTAACTTCTCCTACGACATCAGCAACAAT
GCACCAGGGTATAACAAATGGTTCTTCGTAACGGTGACGAACAATATGATGGGCAATATGAAAATCTACATTAAC
GGGAAACTTATCGACACCATTAAAGTGAAAGAGCTTACTGGGATCAATTTTAGTAAAACCATTACCTTTGAGATC
AACAAAATTCCGGACACGGGTCTGATTACCTCCGATTCGGATAATATCAATATGTGGATTCGCGACTTTTATATC
TTCGCCAAAGAACTTGATGGCAAAGATATCAACATTTTGTTTAATTCCCTGCAGTATACCAATGTCGTTAAGGAC
TATTGGGGCAATGATCTCCGCTACAATAAAGAATACTACATGGTTAACATCGACTATCTCAATCGCTACATGTAT
GCTAACTCGCGTCAAATTGTGTTTAACACACGTCGTAACAACAACGATTTTAACGAAGGTTATAAAATCATTATC
AAACGGATCCGCGGCAATACGAACGATACTCGTGTTCGTGGCGGTGACATTCTGTATTTCGACATGACGATTAAT
AATAAAGCGTACAATCTGTTCATGAAGAACGAAACCATGTACGCCGATAACCATTCCACTGAAGATATCTACGCA
ATCGGACTTCGCGAACAGACCAAAGACATTAACGACAACATCATCTTTCAGATTCAACCGATGAATAATACCTAC
TACTATGCCTCCCAGATCTTCAAAAGTAATTTCAACGGCGAAAACATTTCAGGCATTTGCTCAATCGGCACTTAT
CGGTTCCGGTTAGGTGGTGATTGGTATCGTCACAACTACCTTGTTCCCACAGTGAAACAAGGCAACTATGCATCG
CTCTTAGAAAGCACATCTACGCATTGGGGTTTTGTGCCAGTCAGTGAA
Polypeptide Sequence of rBoNT/C(0)
SEQ ID NO: 12
MPITINNFNYSDPVDNKNILYLDTHLNTLANEPEKAFRITGNIWVIPDRFSRNSNPNLNKPPRVTSPKSGYYDPN
YLSTDSDKDPFLKEIIKLFKRINSREIGEELIYRLSTDIPFPGNNNTPINTFDFDVDFNSVDVKTRQGNNWVKTG
SINPSVIITGPRENIIDPETSTFKLTNNTFAAQEGFGALSIISISPRFMLTYSNATNDVGEGRFSKSEFCMDPIL
ILMGTLNNAMHNLYGIAIPNDQTISSVTSNIFYSQYNVKLEYAEIYAFGGPTIDLIPKSARKYFEEKALDYYRSI
AKRLNSITTANPSSENKYIGEYKQKLIRKYRFVVESSGEVTVNRNKEVELYNELTQIFTEFNYAKIYNVQNRKIY
LSNVYTPVTANILDDNVYDIQNGFNIPKSNLNVLFMGQNLSRNPALRKVNPENMLYLFTKFCHKAIDGRSLYNKT
LDCRELLVKNTDLPFIGDISDVKTDIFLRKDINEETEVIYYPDNVSVDQVILSKNTSEHGQLDLLYPSIDSESEI
LPGENQVFYDNRTQNVDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILLEAFPEFTIPALGAFV
IYSKVQERNEIIKTIDNCLEQRIKRWKDSYEWMMGTWLSRIITQFNNISYQMYDSLNYQAGAIKAKIDLEYKKYS
GSDKENIKSQVENLKNSLDVKISEAMNNINKFIRECSVTYLFKNMLPKVIDELNEFDRNTKAKLINLIDSHNIIL
VGEVDKLKAKVNNSFQNTIPFNIFSYTNNSLLKDIINEYFNNINDSKILSLQNRKNTLVDTSGYNAEVSEEGDVQ
LNPIFPFDFKLGSSGEDRGKVIVTQNENIVYNSMYESFSISFWIRINKWVSNLPGYTIIDSVKNNSGWSIGIISN
FLVFTLKQNEDSEQSINFSYDISNNAPGYNKWFFVTVTNNMMGNMKIYINGKLIDTIKVKELTGINFSKTITFEI
NKIPDTGLITSDSDNINMWIRDFYIFAKELDGKDINILFNSLQYTNVVKDYWGNDLRYNKEYYMVNIDYLNRYMY
ANSRQIVENTRRNNNDFNEGYKIIIKRIRGNTNDTRVRGGDILYFDMTINNKAYNLFMKNETMYADNHSTEDIYA
IGLREQTKDINDNIIFQIQPMNNTYYYASQIFKSNENGENISGICSIGTYRFRLGGDWYRHNYLVPTVKQGNYAS
LLESTSTHWGFVPVSE
Nucleotide Sequence of rBoNT/E(0)
SEQ ID NO: 13
atgccgaaaatcaactctttcaactacaacgacccggttaacgaccgtaccatcctgtatatcaaaccgggtggt
tgccaggagttctacaaatctttcaacatcatgaaaaacatctggatcatcccggaacgtaacgttatcggtacc
accccgcaggacttccacccgccgacctctctgaaaaacggtgactcttcttactacgacccgaactacctccag
tctgacgaagaaaaagaccgtttcctgaaaatcgttaccaaaatcttcaaccgtatcaacaacaacctgtctggt
ggtatcctgctggaagaactgtctaaagctaacccgtacctgggtaacgacaacaccccggacaaccagttccac
atcggtgacgcttctgctgttgaaatcaaattctctaacggttctcaggacatcctgctgccgaacgttatcatc
atgggtgctgaaccggacctgttcgaaaccaactcttctaacatctctctgcgtaacaactacatgccgtctaac
cacggtttcggttctatcgctatcgttaccttctctccggaatactctttccgtttcaacgacaacagcatgaac
gagttcatccaggacccggctctgaccctgatgcaccaactgatctactctctgcacggtctgtacggtgctaaa
ggtatcaccaccaaatacaccatcacccagaaacagaacccgctgatcaccaacatccgtggtaccaacatcgaa
gagttcctgaccttcggtggtaccgacctgaacatcatcacctctgctcagtctaacgacatctacaccaacctg
ctggctgactacaaaaaaatcgcttctaaactgtctaaagttcaggtttctaacccgctgctgaacccgtacaaa
gacgttttcgaagctaaatacggtctggacaaagacgcttctggtatctactctgttaacatcaacaaattcaac
gacatcttcaaaaaactgtactctttcaccgagttcgacctggcgaccaaattccaggttaaatgccgtcagacc
tacatcggtcagtacaaatacttcaaactgtctaacctgctgaacgactctatctacaacatctctgaaggttac
aacatcaacaacctgaaagttaacttccgtggtcagaacgctaacctgaacccgcgtatcatcaccccgatcacc
ggtcgtggtctggttaaaaaaatcatccgtttctgcAAGAATATTGTAAGCGTTAAAGGAATAAGAAAAAGTATC
tgcatcgaaatcaacaacggtgaactgttcttcgttgcttctgaaaactcttacaacgacgacaacatcaacacc
ccgaaagaaatcgacgacaccgttacctctaacaacaactacgaaaacgacctggaccaggttatcctgaacttc
aactctgaatctgctccgggtctgtctgacgaaaaactgaacctgaccatccagaacgacgcttacatcccgaaa
tacgactctaacggtacctctgacatcgaacagcacgacgttaacgaactgaacgttttcttctacctggacgct
cagaaagttccggaaggtgaaaacaacgttaacctgacctcttctatcgacaccgctctgctggaacagccgaaa
atctacaccttcttctcttctgagttcatcaacaacgttaacaaaccggttcaggctgctctgttcgtttcttgg
attcagcaggttctggttgacttcaccaccgaagctaaccagaaatctaccgttgacaaaatcgctgacatctct
atcgttgttccgtacatcggtctggctctgaacatcggtaacgaagctcagaaaggtaacttcaaagacgctctg
gaactgctgggtgctggtatcctgctggagttcgaaccggaactgctgatcccgaccatcctggttttcaccatc
aaatctttcctgggttcttctgacaacaaaaacaaagttatcaaagctatcaacaacgctctgaaagaacgtgac
gaaaaatggaaagaagtttactctttcatcgtttctaactggatgaccaaaatcaacacccagttcaacaaacgt
aaagaacagatgtaccaggctctccagaaccaggttaacgctatcaaaaccatcatcgaatctaaatacaactct
tacaccctggaagaaaaaaacgaactgaccaacaaatacgacatcaaacagatcgaaaacgaactgaaccagaaa
gtttctatcgctatgaacaacatcgaccgtttcctgaccgaatcttctatctcttacctgatgaaactcatcaac
gaagttaaaatcaacaaactgcgtgaatacgacgaaaacgttaaaacctacctgctgaactacatcatccagcac
ggttctatcctgggtgaatctcagcaggaactgaactctatggttaccgacaccctgaacaactctatcccgttc
aaactgtcttcttacaccgacgacaaaatcctGATCTCTTACTTCAACAAATTCTTTAAAcgcATTAAGAGTTCA
TCGGTTctgaatATGCGGTACAAAAATGATAAAtatGTCGATACTTCTGGATATgatAGCAATATCAACATTAAC
GGCGACGTGTATAAATATccgACAAATAAAAACCAGTTTGGGATATATAACGACAAGctgTCGGAGGTCAATatt
TCTCAAAACGACtatATCattTACGATAATaaaTATAAAAACTTTAGCATTAGTtttTGGGTTcgtATACCTAAT
tatGACAATaaaattGTAAATGTGAATAACGAGTATACCATTATAAACTGTATGcgcGACAATAACAGTGGTTGG
AAGGTATCGctgAACCATAATGAGATTATCTGGACCctgcagGATAATgcaGGTATAAACCAGAAACTGGCTTTT
AACTATGGAAACGCAAATGGGATCTCAGATTACATTaataaaTGGatttttGTTaccATTACGAACGATcgcTTA
GGCGACTCAAAACTTTATATTAATggcAATctgATAGATCAGAAATCAATCTTAAATTTGGGCAATATTCATGTC
TCTgatAACATCTTGTTCAAGATCGTTAATTGCAGTTACACTcgtTATATTGGCATTCGTTACTTTAATATCTTC
gataaaGAActgGACGAGACGGAAATCcagACTCTGTATTCAAACGAGCCCAATACTAATATATTGAAAGATTTT
TGGGGTAACTATCTTTTATATGATAAAGAATACTATCTCCTGaatGTATTGAAGCCAAACAATTTCATAGATAGA
CGCAAGGATAGCACATTAAGTATCAACAATATCAGATCTACTATActgttaGCAAATCGCCTcTACTCCggtATT
AAAGTGAAGATTcagCGGGTTAATAACTCCAGTACCAATGATAATCTGGTCCGTAAGAACGATCAGGTATACATC
aatTTCGTCGCGAGCAAAACTcatCTCTTCCCGCTTTACGCCgatACAGCTACGACAAACAAGGAAAAAACCATA
AAAATTTCCAGCTCCGGAAACAGATTCAATCAAGTAGTTGTAATGAACTCTGTGGGTaatAATTGTACGATGAAC
TTTaagAATAACAATGGGAACAATattGGACTTTTGGGCTTcAAAGCCGACACAGTGGTGGCGTCCACCTGGTAT
TACACGcacATGcggGACCATACGAATTCGAACGGTTGCTTCTGGAACTTTATCTCGGAAgaaCACGGGTGGCAA
GAAAAA
Polypeptide Sequence of rBoNT/E(0)
SEQ ID NO: 14
MPKINSFNYNDPVNDRTILYIKPGGCQEFYKSFNIMKNIWIIPERNVIGTTPQDFHPPTSLKNGDSSYYDPNYLQ
SDEEKDRFLKIVTKIFNRINNNLSGGILLEELSKANPYLGNDNTPDNQFHIGDASAVEIKFSNGSQDILLPNVII
MGAEPDLFETNSSNISLRNNYMPSNHGFGSIAIVTFSPEYSFRENDNSMNEFIQDPALTLMHQLIYSLHGLYGAK
GITTKYTITQKQNPLITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLATKFQVKCRQTYIGQYKYFKLSNLLNDSIYNISEGY
NINNLKVNFRGQNANLNPRIITPITGRGLVKKIIRFCKNIVSVKGIRKSICIEINNGELFFVASENSYNDDNINT
PKEIDDTVTSNNNYENDLDQVILNENSESAPGLSDEKLNLTIQNDAYIPKYDSNGTSDIEQHDVNELNVFFYLDA
QKVPEGENNVNLTSSIDTALLEQPKIYTFFSSEFINNVNKPVQAALFVSWIQQVLVDETTEANQKSTVDKIADIS
IVVPYIGLALNIGNEAQKGNEKDALELLGAGILLEFEPELLIPTILVFTIKSFLGSSDNKNKVIKAINNALKERD
EKWKEVYSFIVSNWMTKINTQFNKRKEQMYQALQNQVNAIKTIIESKYNSYTLEEKNELTNKYDIKQIENELNQK
VSIAMNNIDRELTESSISYLMKLINEVKINKLREYDENVKTYLLNYIIQHGSILGESQQELNSMVTDTLNNSIPF
KLSSYTDDKILISYFNKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDQKSILNLGNIHVSDNILFKIVNCSYTRYIGIRYFNIF
DKELDETEIQTLYSNEPNTNILKDFWGNYLLYDKEYYLLNVLKPNNFIDRRKDSTLSINNIRSTILLANRLYSGI
KVKIQRVNNSSTNDNLVRKNDQVYINFVASKTHLFPLYADTATTNKEKTIKISSSGNRFNQVVVMNSVGNNCTMN
FKNNNGNNIGLLGFKADTVVASTWYYTHMRDHTNSNGCFWNFISEEHGWQEK
Nucleotide Sequence of rBoNT/F(0)
SEQ ID NO: 15
ATGCCGGTGGTCATCAACAGCTTCAACTACAACGACCCAGTAAACGACGACACGATCCTGTATATGCAAATCCCG
TATGAAGAGAAGAGCAAGAAGTACTATAAGGCCTTTGAAATCATGCGCAATGTGTGGATTATTCCGGAGCGTAAT
ACGATTGGTACTGACCCAAGCGACTTCGATCCACCTGCGTCTTTGGAAAACGGCTCGTCCGCATATTACGACCCG
AATTACCTGACCACCGATGCGGAGAAAGATCGTTATTTGAAAACCACCATCAAGCTGTTCAAACGCATTAACAGC
AATCCGGCAGGTGAGGTCCTGCTGCAAGAGATTAGCTACGCAAAGCCTTATCTGGGTAATGAGCATACGCCTATT
AACGAGTTTCACCCGGTTACCCGCACTACCAGCGTTAACATCAAGTCCTCGACCAACGTGAAGTCTAGCATTATC
CTGAACCTGCTGGTTCTGGGTGCCGGTCCGGACATCTTCGAAAACTCTAGCTACCCGGTGCGTAAACTGATGGAT
AGCGGCGGTGTTTATGACCCGAGCAATGACGGTTTTGGCAGCATCAATATCGTGACGTTTAGCCCGGAGTACGAG
TACACCTTCAATGATATCAGCGGTGGTTACAATTCTTCTACCGAGAGCTTCATCGCCGACCCGGCGATCAGCCTG
GCACACCAACTGATCTATGCATTGCATGGCTTGTACGGTGCCCGTGGTGTGACGTATAAAGAGACTATCAAGGTT
AAGCAGGCACCTCTGATGATTGCGGAAAAGCCGATTCGCCTGGAAGAGTTCCTGACCTTCGGCGGTCAAGATTTG
AACATCATTACCTCGGCCATGAAAGAGAAAATCTATAACAATTTGCTGGCCAACTATGAAAAGATTGCAACGCGC
TTGTCTCGTGTTAACTCCGCTCCGCCGGAATACGACATTAATGAGTACAAAGACTACTTTCAATGGAAATATGGC
CTGGACAAAAATGCGGATGGTTCTTATACCGTGAATGAAAACAAATTCAATGAAATCTACAAGAAACTGTACAGC
TTCACCGAAATCGATCTGGCGAACAAGTTCAAAGTCAAATGTCGTAATACCTACTTCATCAAATATGGCTTCCTG
AAAGTCCCGAACCTGCTGGACGATGACATCTATACCGTCAGCGAAGGCTTCAACATCGGCAATCTGGCCGTGAAT
AATCGTGGTCAGAACATCAAACTGAATCCGAAAATCATTGACTCCATCCCAGACAAGGGCCTGGTTGAGAAAATC
GTGAAGTTCTGCAAAAGCGTTATTCCGCGTAAAGGTACGAAAGCACCGCCTCGCCTGTGCATTCGCGTTAACAAC
CGTGAGTTGTTCTTTGTGGCATCTGAAAGCAGCTACAACGAGAACGACATCAACACCCCTAAAGAAATTGATGAT
ACCACGAACCTGAATAACAATTATCGCAACAATCTGGACGAGGTGATCCTGGATTACAATTCGGAAACCATTCCG
CAAATTAGCAATCAGACGCTGAACACCCTGGTTCAGGACGATAGCTACGTTCCGCGTTACGACTCCAATGGTACT
AGCGAGATTGAAGAACACAACGTAGTGGACTTGAACGTTTTCTTTTATCTGCACGCCCAGAAGGTTCCGGAGGGC
GAAACCAATATTAGCCTGACCAGCTCGATCGACACCGCGCTGTCTGAGGAGAGCCAAGTCTACACCTTTTTCAGC
AGCGAGTTTATCAACACTATTAACAAGCCAGTTCATGCTGCATTGTTTATCTCTTGGATTAACCAGGTGATTCGC
GACTTTACGACGGAGGCGACCCAGAAGTCTACCTTCGACAAAATTGCAGACATCTCCCTGGTCGTCCCATACGTC
GGCCTGGCGTTGAATATTGGCAATGAAGTTCAAAAAGAGAACTTCAAAGAAGCGTTCGAGCTGCTGGGTGCAGGC
ATCCTGCTGGAGTTCGTGCCGGAACTGTTGATCCCGACCATCCTGGTGTTCACCATTAAGAGCTTCATTGGATCC
TCCGAGAATAAGAACAAGATCATCAAGGCGATCAATAACAGCCTGATGGAGCGTGAAACGAAGTGGAAAGAAATC
TATAGCTGGATTGTTAGCAATTGGCTGACTCGTATTAACACGCAATTCAACAAGCGTAAAGAGCAAATGTACCAA
GCCCTGCAAAACCAAGTTGACGCCATCAAAACGGTAATTGAATACAAGTACAACAATTACACGAGCGATGAGCGC
AACCGCCTGGAAAGCGAATACAACATCAACAACATTCGCGAAGAATTGAACAAGAAAGTGAGCCTGGCGATGGAG
AACATTGAGCGTTTTATCACCGAAAGCAGCATCTTTTACCTGATGAAATTGATTAATGAGGCGAAAGTCTCGAAA
CTGCGTGAGTACGACGAAGGTGTGAAAGAGTATCTGCTGGATTACATTAGCGAGCACCGTAGCATCTTGGGTAAC
TCGGTTCAGGAGCTGAACGATCTGGTGACCTCTACCCTGAACAATAGCATCCCGTTCGAACTGAGCAGCTATACC
AATGACAAGATTCTGATTCTGTATTTCAATAAACTGTATAAGAAGATCAAGGATAACAGCATTCTGGATATGCGT
TACGAAAACAATAAGTTTATCGACATTTCTGGTTACGGCAGCAACATTTCCATCAATGGCGATGTCTACATCTAC
AGCACCAATCGCAACCAGTTCGGCATCTACTCTAGCAAACCGAGCGAAGTTAACATCGCACAGAACAATGATATT
ATTTATAACGGTCGTTATCAAAACTTCTCTATCAGCTTTTGGGTCCGTATCCCGAAGTACTTCAATAAAGTCAAT
CTGAATAATGAATACACGATCATCGACTGCATTCGCAATAACAACAGCGGTTGGAAAATCAGCCTGAATTACAAC
AAAATTATTTGGACCCTGCAAGATACGGCGGGTAACAATCAGAAACTGGTGTTTAACTACACGCAAATGATCAGC
ATTTCTGACTATATCAACAAGTGGATCTTTGTTACCATCACCAATAATCGTCTGGGCAATAGCCGTATTTACATC
AACGGTAACCTGATTGATGAGAAAAGCATCAGCAACCTGGGCGATATTCACGTCAGCGACAACATTCTGTTCAAA
ATTGTTGGTTGTAACGATACCCGTTACGTCGGCATCCGTTATTTCAAGGTTTTCGATACGGAGCTGGGTAAAACG
GAAATCGAAACGTTGTACTCCGATGAACCAGATCCGAGCATTCTGAAGGACTTTTGGGGTAACTACTTGCTGTAC
AATAAACGTTACTATCTGCTGAATCTGTTGCGCACCGACAAGAGCATTACCCAAAACAGCAATTTCCTGAACATT
AATCAGCAACGCGGCGTATACCAAAAACCGAACATCTTCAGCAATACGCGCCTGTATACTGGTGTTGAAGTGATC
ATTCGTAAGAACGGTAGCACCGACATTAGCAACACGGACAATTTCGTCCGTAAGAATGACCTGGCGTACATTAAC
GTCGTGGACCGTGATGTCGAGTATCGTCTGTACGCAGACATCAGCATTGCGAAACCGGAAAAGATTATCAAGCTG
ATCCGTACCAGCAACAGCAACAACAGCCTGGGTCAGATCATTGTGATGGACAGCATTGGTAATAACTGCACGATG
AACTTCCAGAACAACAATGGTGGTAATATCGGTCTGCTGGGTTTTCACAGCAATAATCTGGTTGCTTCCAGCTGG
TACTACAATAACATTCGTAAAAACACGTCTAGCAATGGTTGTTTTTGGAGCTTTATCAGCAAAGAGCACGGCTGG
CAAGAAAAT
Polypeptide Sequence of rBoNT/F(0)
SEQ ID NO: 16
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHQLIYALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTEDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYENKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDCIRNNNSGWKISLNYN
KIIWTLQDTAGNNQKLVFNYTQMISISDYINKWIFVTITNNRLGNSRIYINGNLIDEKSISNLGDIHVSDNILFK
IVGCNDTRYVGIRYFKVFDTELGKTEIETLYSDEPDPSILKDFWGNYLLYNKRYYLLNLLRTDKSITQNSNFLNI
NQQRGVYQKPNIFSNTRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTSSNGCFWSFISKEHGW
QEN
Nucleotide Sequence of rBoNT/A(0) (His-tagged)
SEQ ID NO: 17
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAAAGCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACACCAGCTG
ATTTATGCCGGTCATCGTCTGTATGGTATTGCCATTAATCCGAATCGTGTGTTCAAAGTGAATACCAACGCCTAT
TATGAAATGAGCGGTCTGGAAGTGAGTTTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTCGAATTT
TACAAACTGCTGTGTGTTCGTGGCATTATTACCAGCAAAACCAAAAGTCTGGATAAAGGCTACAATAAAGCCCTG
AATGATCTGTGCATTAAGGTGAATAATTGGGACCTGTTTTTTAGCCCGAGCGAGGATAATTTCACCAACGATCTG
AACAAAGGCGAAGAAATTACCAGCGATACCAATATTGAAGCAGCCGAAGAAAACATTAGCCTGGATCTGATTCAG
CAGTATTATCTGACCTTCAACTTCGATAATGAGCCGGAAAATATCAGCATTGAAAACCTGAGCAGCGATATTATT
GGCCAGCTGGAACTGATGCCGAATATTGAACGTTTTCCGAACGGCAAAAAATACGAGCTGGATAAATACACCATG
TTCCATTATCTGCGTGCCCAAGAATTTGAACATGGTAAAAGCCGTATTGCACTGACCAATAGCGTTAATGAAGCA
CTGCTGAACCCGAGCCGTGTTTATACCTTTTTTAGCAGCGATTACGTGAAAAAGGTTAACAAAGCAACCGAAGCA
GCCATGTTTTTAGGTTGGGTTGAACAGCTGGTTTATGATTTCACCGATGAAACCAGCGAAGTTAGCACCACCGAT
AAAATTGCAGATATTACCATCATCATCCCGTATATCGGTCCGGCACTGAATATTGGCAATATGCTGTATAAAGAC
GATTTTGTGGGTGCCCTGATTTTTAGCGGTGCAGTTATTCTGCTGGAATTTATTCCGGAAATTGCCATTCCGGTT
CTGGGCACCTTTGCACTGGTGAGCTATATTGCAAATAAAGTTCTGACCGTGCAGACCATCGATAATGCACTGAGC
AAACGTAACGAAAAATGGGATGAAGTGTACAAGTATATCGTGACCAATTGGCTGGCAAAAGTTAACACCCAGATT
GACCTGATTCGCAAGAAGATGAAAGAAGCACTGGAAAATCAGGCAGAAGCAACCAAAGCCATTATCAACTATCAG
TATAACCAGTACACCGAAGAAGAGAAAAATAACATCAACTTCAACATCGACGATCTGTCCAGCAAACTGAACGAA
AGCATCAACAAAGCCATGATTAACATTAACAAATTTCTGAACCAGTGCAGCGTGAGCTATCTGATGAATAGCATG
ATTCCGTATGGTGTGAAACGTCTGGAAGATTTTGATGCAAGCCTGAAAGATGCCCTGCTGAAATATATCTATGAT
AATCGTGGCACCCTGATTGGTCAGGTTGATCGTCTGAAAGATAAAGTGAACAACACCCTGAGTACCGATATTCCT
TTTCAGCTGAGCAAATATGTGGATAATCAGCGTCTGCTGTCAACCTTTACCGAATACATTAAGAACATCATCAAC
ACCAGCATTCTGAACCTGCGTTATGAAAGCAATCATCTGATTGATCTGAGCCGTTATGCCAGCAAAATCAATATA
GGCAGCAAGGTTAACTTCGACCCGATTGACAAAAATCAGATACAGCTGTTTAATCTGGAAAGCAGCAAAATTGAG
GTGATCCTGAAAAACGCCATTGTGTATAATAGCATGTACGAGAATTTCTCGACCAGCTTTTGGATTCGTATCCCG
AAATACTTTAATAGCATCAGCCTGAACAACGAGTACACCATTATTAACTGCATGGAAAACAATAGCGGCTGGAAA
GTTAGCCTGAATTATGGCGAAATTATCTGGACCCTGCAGGATACCCAAGAAATCAAACAGCGTGTGGTTTTCAAA
TACAGCCAGATGATTAATATCAGCGACTATATCAACCGCTGGATTTTTGTGACCATTACCAATAATCGCCTGAAT
AACAGCAAGATCTATATTAACGGTCGTCTGATTGACCAGAAACCGATTAGTAATCTGGGTAATATTCATGCGAGC
AACAACATCATGTTTAAACTGGATGGTTGTCGTGATACCCATCGTTATATTTGGATCAAGTACTTCAACCTGTTC
GATAAAGAGTTGAACGAAAAAGAAATTAAAGACCTGTATGATAACCAGAGCAACAGCGGTATTCTGAAGGATTTT
TGGGGAGATTATCTGCAGTATGACAAACCGTATTATATGCTGAATCTGTACGACCCGAATAAATACGTGGATGTG
AATAATGTTGGCATCCGTGGTTATATGTACCTGAAAGGTCCGCGTGGTAGCGTTATGACCACAAACATTTATCTG
AATAGCAGCCTGTATCGCGGAACCAAATTCATCATTAAAAAGTATGCCAGCGGCAACAAGGATAATATTGTGCGT
AATAATGATCGCGTGTACATTAACGTTGTGGTGAAGAATAAAGAATATCGCCTGGCAACCAATGCAAGCCAGGCA
GGCGTTGAAAAAATTCTGAGTGCCCTGGAAATTCCGGATGTTGGTAATCTGAGCCAGGTTGTTGTGATGAAAAGC
AAAAATGATCAGGGCATCACCAACAAGTGCAAAATGAATCTGCAGGACAATAACGGCAACGATATTGGTTTTATT
GGCTTCCACCAGTTCAACAATATTGCGAAACTGGTTGCAAGCAATTGGTATAATCGTCAGATTGAACGTAGCAGT
CGTACCCTGGGTTGTAGCTGGGAATTTATCCCTGTGGATGATGGTTGGGGTGAACGTCCGCTGGAAAACCTGTAT
TTTCAAGGTGCAAGTCATCATCACCATCACCACCATCATTAA
Polypeptide Sequence of rBoNT/A(0) (His-tagged)
SEQ ID NO: 18
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLENLESSKIEVILKNAIVYNSMYENFSTSFWIRIPKYENSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQENNIAKLVASNWYNRQIERSS
RTLGCSWEFIPVDDGWGERPLENLYFQGASHHHHHHHH
Nucleotide Sequence of rLH_{N}/A (His-tagged)
SEQ ID NO: 19
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAATCCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACATGAACTG
ATTCATGCCGGTCATCGTCTGTATGGTATTGCAATTAATCCGAACCGTGTGTTCAAAGTGAATACCAACGCATAT
TATGAAATGAGCGGTCTGGAAGTGTCATTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTCGAATTT
TACAAACTGCTGTGTGTTCGTGGCATTATTACCAGCAAAACCAAAAGTCTGGATAAAGGCTACAATAAAGCCCTG
AATGATCTGTGCATTAAGGTGAATAATTGGGACCTGTTTTTTAGCCCGAGCGAGGATAATTTCACCAACGATCTG
AACAAAGGCGAAGAAATTACCAGCGATACCAATATTGAAGCAGCCGAAGAAAACATTAGCCTGGATCTGATTCAG
CAGTATTATCTGACCTTCAACTTCGATAATGAGCCGGAAAATATCAGCATTGAAAACCTGAGCAGCGATATTATT
GGCCAGCTGGAACTGATGCCGAATATTGAACGTTTTCCGAACGGCAAAAAATACGAGCTGGATAAATACACCATG
TTCCATTATCTGCGTGCCCAAGAATTTGAACATGGTAAAAGCCGTATTGCACTGACCAATAGCGTTAATGAAGCA
CTGCTGAACCCGAGCCGTGTTTATACCTTTTTTAGCAGCGATTACGTGAAAAAGGTTAACAAAGCAACCGAAGCA
GCCATGTTTTTAGGTTGGGTTGAACAGCTGGTTTATGATTTCACCGATGAAACCAGCGAAGTTAGCACCACCGAT
AAAATTGCAGATATTACCATCATCATCCCGTATATCGGTCCGGCACTGAATATTGGCAATATGCTGTATAAAGAC
GATTTTGTGGGTGCCCTGATTTTTAGCGGTGCAGTTATTCTGCTGGAATTTATTCCGGAAATTGCCATTCCGGTT
CTGGGCACCTTTGCACTGGTGAGCTATATTGCAAATAAAGTTCTGACCGTGCAGACCATCGATAATGCACTGAGC
AAACGTAACGAAAAATGGGATGAAGTGTACAAGTATATCGTGACCAATTGGCTGGCAAAAGTTAACACCCAGATT
GACCTGATTCGCAAGAAGATGAAAGAAGCACTGGAAAATCAGGCAGAAGCAACCAAAGCCATTATCAACTATCAG
TATAACCAGTACACCGAAGAAGAGAAAAATAACATCAACTTCAACATCGACGATCTGTCCAGCAAACTGAACGAA
AGCATCAACAAAGCCATGATTAACATTAACAAATTTCTGAACCAGTGCAGCGTGAGCTATCTGATGAATAGCATG
ATTCCGTATGGTGTGAAACGTCTGGAAGATTTTGATGCAAGCCTGAAAGATGCCCTGCTGAAATATATCTATGAT
AATCGTGGCACCCTGATTGGTCAGGTTGATCGTCTGAAAGATAAAGTGAACAACACCCTGAGTACCGATATTCCT
TTTCAGCTGAGCAAATATGTGGATAATCAGCGTCTGCTGTCAACCGAAAATCTGTATTTCCAGGGTGCAAGTCAT
CATCACCATCACCACCATCATTAA
Polypeptide Sequence of rLH_{N}/A (His-tagged)
SEQ ID NO: 20
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTENLYFQGASHHHHHHHH
Nucleotide Sequence of rH_{C}/A (His-tagged)
SEQ ID NO: 21
ATGCATCATCACCATCACCACGAAAATCTATACTTCCAAGGAAAAAACATCATCAATACTAGCATTCTGAACCTG
CGTTACGAGAGCAATCATCTGATTGATCTGAGCCGTTATGCAAGCAAGATCAACATCGGTAGCAAGGTCAATTTT
GACCCGATCGATAAGAACCAGATCCAGCTGTTTAATCTGGAATCGAGCAAAATTGAGGTTATCCTGAAAAACGCC
ATTGTCTACAACTCCATGTACGAGAATTTCTCCACCAGCTTCTGGATTCGCATCCCGAAATACTTCAACAGCATT
AGCCTGAACAACGAGTATACTATCATCAACTGTATGGAGAACAACAGCGGTTGGAAGGTGTCTCTGAACTATGGT
GAGATCATTTGGACCTTGCAGGACACCCAAGAGATCAAGCAGCGCGTCGTGTTCAAGTACTCTCAAATGATCAAC
ATTTCCGATTACATTAATCGTTGGATCTTCGTGACCATTACGAATAACCGTCTGAATAACAGCAAGATTTACATC
AATGGTCGCTTGATCGATCAGAAACCGATTAGCAACCTGGGTAATATCCACGCAAGCAACAACATTATGTTCAAA
TTGGACGGTTGCCGCGATACCCATCGTTATATCTGGATCAAGTATTTCAACCTGTTTGATAAAGAACTGAATGAG
AAGGAGATCAAAGATTTGTATGACAACCAATCTAACAGCGGCATTTTGAAGGACTTCTGGGGCGATTATCTGCAA
TACGATAAGCCGTACTATATGCTGAACCTGTATGATCCGAACAAATATGTGGATGTCAATAATGTGGGTATTCGT
GGTTACATGTATTTGAAGGGTCCGCGTGGCAGCGTTATGACGACCAACATTTACCTGAACTCTAGCCTGTACCGT
GGTACGAAATTCATCATTAAGAAATATGCCAGCGGCAACAAAGATAACATTGTGCGTAATAACGATCGTGTCTAC
ATCAACGTGGTCGTGAAGAATAAAGAGTACCGTCTGGCGACCAACGCTTCGCAGGCGGGTGTTGAGAAAATTCTG
AGCGCGTTGGAGATCCCTGATGTCGGTAATCTGAGCCAAGTCGTGGTTATGAAGAGCAAGAACGACCAGGGTATC
ACTAACAAGTGCAAGATGAACCTGCAAGACAACAATGGTAACGACATCGGCTTTATTGGTTTCCACCAGTTCAAC
AATATTGCTAAACTGGTAGCGAGCAATTGGTACAATCGTCAGATTGAGCGCAGCAGCCGTACTTTGGGCTGTAGC
TGGGAGTTTATCCCGGTCGATGATGGTTGGGGCGAACGTCCGCTGTAA
Polypeptide Sequence of rH_{C}/A (His-tagged)
SEQ ID NO: 22
MHHHHHHENLYFQGKNIINTSILNLRYESNHLIDLSRYASKINIGSKVNFDPIDKNQIQLENLESSKIEVILKNA
IVYNSMYENFSTSFWIRIPKYFNSISLNNEYTIINCMENNSGWKVSLNYGEIIWTLQDTQEIKQRVVFKYSQMIN
ISDYINRWIFVTITNNRLNNSKIYINGRLIDQKPISNLGNIHASNNIMFKLDGCRDTHRYIWIKYFNLEDKELNE
KEIKDLYDNQSNSGILKDFWGDYLQYDKPYYMLNLYDPNKYVDVNNVGIRGYMYLKGPRGSVMTTNIYLNSSLYR
GTKFIIKKYASGNKDNIVRNNDRVYINVVVKNKEYRLATNASQAGVEKILSALEIPDVGNLSQVVVMKSKNDQGI
TNKCKMNLQDNNGNDIGFIGFHQFNNIAKLVASNWYNRQIERSSRTLGCSWEFIPVDDGWGERPL
Nucleotide Sequence of rLC/A (His-tagged)
SEQ ID NO: 23
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAATCCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACATGAACTG
ATTCATGCCGGTCATCGTCTGTATGGTATTGCAATTAATCCGAACCGTGTGTTCAAAGTGAATACCAACGCATAT
TATGAAATGAGCGGTCTGGAAGTGTCATTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTTGAAGAG
AATCTGTATTTCCAGGGTGCAAGTCATCATCACCATCACCACCATCATTAA
Polypeptide Sequence of rLC/A (His-tagged)
SEQ ID NO: 24
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEENLYFQGASHHHHHHHH
Nucleotide Sequence of rBoNT/FA(0) (His-tagged)
SEQ ID NO: 25
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CACCAGCTGATTTATGTTCTGCATGGTCTGTATGGCACCTATATCACCGAATACAAAATTACCCCGAATGTGGTT
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCAATACCAAAAACAGCCTGTGCATTACCGTTAATAATCGCGACCTGTTTTTTATCGCA
AGCCAAGAAAGCTATGGCGAGAATACCATTAACACCTATAAAGAGATTGACGATACCACCACACTGGATCCGAGC
TTTGAAGATATTCTGGATAAAGTGATCCTGAACTTCAACGAACAGGTTATTCCGCAGATGCCGAATCGTAATGTT
AGCACCGATATTCAGAAAGACAACTACATCCCGAAATACGATTATAACCGCACCGACATTATCGATAGCTATGAA
GTTGGTCGCAACTACAACACCTTTTTCTATCTGAATGCCCAGAAATTTAGCCCGAACGAAAGCAATATTACCCTG
ACCAGCAGCTTTGATACAGGTCTGTTAGAAGGTAGCAAAGTGTATACCTTTTTCAGCAGCGATTTCATTAACAAC
ATCAACAAACCGGTTCAGGCCCTGCTGTTTATTGAATGGGTTAAACAGGTGATTCGCGATTTTACCACCGAAGCA
ACCAAAACCTCAACCGTTGATAAACTGAAAGATATTAGCCTGGTGGTGCCGTATATTGGTCTGGCACTGAATATT
GGTGATGAGATCTACAAACAGCATTTTGCAGAAGCAGTTGAACTGGTTGGTGCAGGTCTGCTGCTGGAATTTTCA
CCGGAATTTCTTATTCCGACGCTGCTGATTTTTACCATCAAAGGTTATCTGACCGGTAGCATTCGCGATAAAGAC
AAAATCATTAAAACCCTGGATAACGCCCTGAATGTTCGTGATCAGAAATGGAAAGAACTGTATCGTTGGGTTGTT
AGCAAATGGCTGACCACCATTAATACGCAGTTCAACAAACGCAAAGAACAAATGTACAAAGCCCTGAAAAATCAG
GCCACCGCCATTAAAAAGATCATCGAGAACAAATATAACAACTATACCACCGATGAAAAAAGCAAGATCGATAGC
AGCTATAACATCAACGAAATTGAACGCACCCTGAACGAAAAAATCAATCTGGCCATGAAAAACATCGAGCAGTTT
ATTACCGAAAGCAGCATTGCCTATCTGATCAATATCATCAACAACGAAACGATCCAGAAACTGAAAAGCTATGAT
GACCTGGTTCGTCGTTATCTGCTGGGTTATATTCGTAATCATAGCAGCATTCTGGGCAATAGCGTTGAAGAACTG
AATTCCAAAGTGAACAACCATCTGGATAATGGCATTCCGTTTGAACTGAGCAGTTATACCAATGATAGCCTGCTG
ATCCGCTACTTCAATAAAAACTATGGCGAACTGAAGTACAACTGCATTCTGAACATCAAATATGAGATGGATCGT
GACAAACTGGTTGATAGCAGCGGTTATCGTAGCCGTATCAATATTGGTACAGGCGTCAAATTTAGCGAGATCGAT
AAAAATCAAGTGCAGCTGAGCAATCTGGAATCCAGCAAAATTGAAGTCATTCTGAATAACGGCGTCATCTATAAC
AGCATGTATGAAAACTTTTCGACCAGCTTTTGGATTCGCATTCCGAAATACTTTCGCAACATCAATAACGAGTAC
AAGATCATCAGCTGTATGCAGAATAATAGCGGTTGGGAAGTGAGCCTGAATTTTAGCAATATGAACTCGAAAATC
ATCTGGACCCTGCAGGATACCGAAGGTATCAAAAAAACCGTTGTGTTTCAGTACACCCAGAACATTAACATTAGC
GACTATATCAACCGCTGGATCTTTGTGACCATTACAAATAATCGTCTGAGCAACAGCAAAATCTACATTAATGGT
CGCCTGATCAACGAAGAAAGCATTAGCGATCTGGGTAATATCCATGCCAGCAACAACATTATGTTTAAACTGGAT
GGTTGCCGTGATCCGCATCGTTATATCTGGATTAAATACTTTAACCTGTTTGACAAAGAGCTGAACAAGAAAGAA
ATTAAAGATCTGTACGACAACCAGAGCAATAGCGGTATTCTGAAAGATTTCTGGGGTGATTATCTGCAGTATGAC
AAACCGTATTATATGCTGAATCTGTATGACCCGAATAAGTATCTGGATGTGAATAATGTTGGCATCCGTGGCTAT
ATGTATCTGAAAGGTCCGCGTGGTCGTATTGTGACCACCAACATTTATCTGAATAGCACCCTGTATATGGGCACC
AAATTCATCATTAAGAAATATGCCAGCGGCAACAAAGATAACATTGTGCGTAATAATGATCGCGTGTATATTAAC
GTGGTGGTGAAGAATAAAGAATATCGCCTGGCAACCAATGCAAGCCAGGCAGGCGTTGAAAAAATTCTGAGCGCA
GTTGAAATCCCGGATGTTGGTAATCTGAGCCAGGTTGTTGTGATGAAAAGCGAAAATGATCAGGGCATTCGCAAC
AAGTGTAAAATGAATCTGCAAGACAATAACGGCAACGATATTGGCTTTATCGGCTTTCACCAGTTTAATAACATT
GCAAAACTGGTGGCCAGCAACTGGTATAACCGTCAGATTGGTAAAGCAAGCCGTACCTTTGGTTGTAGCTGGGAA
TTTATCCCGGTTGATGATGGTTGGGGTGAAAGCAGCCTGGAAAATCTGTATTTCCAGGGTGCCAGTCATCATCAC
CACCATCACCATCACTGA
Polypeptide Sequence of rBoNT/FA(0) (His-tagged)
SEQ ID NO: 26
MPVVINSFNYDDPVNDNTIIYIRPPYYETSNTYFKAFQIMDNVWIIPERYRIGIDPSLENPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HQLIYVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGENIGALSINYQGQNIGSDINSIKKLQGQGVVSRVVRLCSNSNTKNSLCITVNNRDLFFIA
SQESYGENTINTYKEIDDTTTLDPSFEDILDKVILNFNEQVIPQMPNRNVSTDIQKDNYIPKYDYNRTDIIDSYE
VGRNYNTFFYLNAQKFSPNESNITLTSSFDTGLLEGSKVYTFFSSDFINNINKPVQALLFIEWVKQVIRDETTEA
TKTSTVDKLKDISLVVPYIGLALNIGDEIYKQHFAEAVELVGAGLLLEFSPEFLIPTLLIFTIKGYLTGSIRDKD
KIIKTLDNALNVRDQKWKELYRWVVSKWLTTINTQFNKRKEQMYKALKNQATAIKKIIENKYNNYTTDEKSKIDS
SYNINEIERTLNEKINLAMKNIEQFITESSIAYLINIINNETIQKLKSYDDLVRRYLLGYIRNHSSILGNSVEEL
NSKVNNHLDNGIPFELSSYTNDSLLIRYFNKNYGELKYNCILNIKYEMDRDKLVDSSGYRSRINIGTGVKFSEID
KNQVQLSNLESSKIEVILNNGVIYNSMYENFSTSFWIRIPKYFRNINNEYKIISCMQNNSGWEVSLNFSNMNSKI
IWTLQDTEGIKKTVVFQYTQNINISDYINRWIFVTITNNRLSNSKIYINGRLINEESISDLGNIHASNNIMFKLD
GCRDPHRYIWIKYFNLFDKELNKKEIKDLYDNQSNSGILKDFWGDYLQYDKPYYMLNLYDPNKYLDVNNVGIRGY
MYLKGPRGRIVTTNIYLNSTLYMGTKFIIKKYASGNKDNIVRNNDRVYINVVVKNKEYRLATNASQAGVEKILSA
VEIPDVGNLSQVVVMKSENDQGIRNKCKMNLQDNNGNDIGFIGFHQFNNIAKLVASNWYNRQIGKASRTFGCSWE
FIPVDDGWGESSLENLYFQGASHHHHHHHH
Nucleotide Sequence of rLH_{N}/FA (His-tagged)
SEQ ID NO: 27
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CATGAACTGATTCATGTTCTGCATGGTCTGTATGGCACCTATATTACCGAATACAAAATTACCCCGAATGTGGTG
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCAATACCAAAAACAGCCTGTGCATTACCGTTAATAATCGCGACCTGTTTTTTATCGCA
AGCCAAGAAAGCTATGGCGAGAATACCATTAACACCTATAAAGAGATTGACGATACCACCACACTGGATCCGAGC
TTTGAAGATATTCTGGATAAAGTGATCCTGAACTTCAACGAACAGGTTATTCCGCAGATGCCGAATCGTAATGTT
AGCACCGATATTCAGAAAGACAACTACATCCCGAAATACGATTATAACCGCACCGACATTATCGATAGCTATGAA
GTTGGTCGCAACTACAACACCTTTTTCTATCTGAATGCCCAGAAATTTAGCCCGAACGAAAGCAATATTACCCTG
ACCAGCAGCTTTGATACAGGTCTGTTAGAAGGTAGCAAAGTGTATACCTTTTTCAGCAGCGATTTCATTAACAAC
ATCAACAAACCGGTTCAGGCCCTGCTGTTTATTGAATGGGTTAAACAGGTGATTCGCGATTTTACCACCGAAGCA
ACCAAAACCTCAACCGTTGATAAACTGAAAGATATTAGCCTGGTGGTGCCGTATATTGGTCTGGCACTGAATATT
GGTGATGAGATCTACAAACAGCATTTTGCAGAAGCAGTTGAACTGGTTGGTGCAGGTCTGCTGCTGGAATTTTCA
CCGGAATTTCTTATTCCGACGCTGCTGATTTTTACCATCAAAGGTTATCTGACCGGTAGCATTCGCGATAAAGAC
AAAATCATTAAAACCCTGGATAACGCCCTGAATGTTCGTGATCAGAAATGGAAAGAACTGTATCGTTGGGTTGTT
AGCAAATGGCTGACCACCATTAATACGCAGTTCAACAAACGCAAAGAACAAATGTACAAAGCCCTGAAAAATCAG
GCCACCGCCATTAAAAAGATCATCGAGAACAAATATAACAACTATACCACCGATGAAAAAAGCAAGATCGATAGC
AGCTATAACATCAACGAAATTGAACGCACCCTGAACGAAAAAATCAATCTGGCCATGAAAAACATCGAGCAGTTT
ATTACAGAAAGCAGCATTGCCTACCTGATCAATATCATCAACAACGAAACCATTCAGAAACTGAAAAGCTATGAT
GACCTGGTTCGTCGTTATCTGCTGGGTTATATTCGTAATCATAGCAGCATTCTGGGCAATAGCGTTGAAGAACTG
AATTCCAAAGTGAACAACCATCTGGATAATGGCATTCCGTTTGAACTGAGCAGTTATACCAATGATAGCCTGCTG
ATCCGCTACTTCAATAAAAACTATGGCGAAGAGAACCTGTATTTCCAGGGTGCCAGTCATCATCACCACCATCAC
CATCACTGA
Polypeptide Sequence of rLH_{N}/FA (His-tagged)
SEQ ID NO: 28
MPVVINSFNYDDPVNDNTIIYIRPPYYETSNTYFKAFQIMDNVWIIPERYRLGIDPSLENPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HELIHVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGENIGALSINYQGQNIGSDINSIKKLQGQGVVSRVVRLCSNSNTKNSLCITVNNRDLFFIA
SQESYGENTINTYKEIDDTTTLDPSFEDILDKVILNFNEQVIPQMPNRNVSTDIQKDNYIPKYDYNRTDIIDSYE
VGRNYNTFFYLNAQKESPNESNITLTSSFDTGLLEGSKVYTFFSSDFINNINKPVQALLFIEWVKQVIRDETTEA
TKTSTVDKLKDISLVVPYIGLALNIGDEIYKQHFAEAVELVGAGLLLEFSPEFLIPTLLIFTIKGYLTGSIRDKD
KIIKTLDNALNVRDQKWKELYRWVVSKWLTTINTQFNKRKEQMYKALKNQATAIKKIIENKYNNYTTDEKSKIDS
SYNINEIERTLNEKINLAMKNIEQFITESSIAYLINIINNETIQKLKSYDDLVRRYLLGYIRNHSSILGNSVEEL
NSKVNNHLDNGIPFELSSYTNDSLLIRYFNKNYGEENLYFQGASHHHHHHHH
Nucleotide Sequence of rH_{C}/FA (His-tagged)
SEQ ID NO: 29
ATGCTGAAGTATAACTGCATCCTGAACATCAAATATGAGATGGATCGTGATAAACTGGTTGATAGCAGCGGTTAT
CGTAGCCGTATCAATATTGGCACCGGTGTGAAATTTAGCGAGATCGATAAAAATCAGGTGCAGCTGAGCAATCTG
GAAAGCAGCAAAATTGAAGTGATTCTGAATAACGGCGTGATCTACAATAGCATGTATGAAAACTTTTCGACCAGC
TTCTGGATTCGCATTCCGAAATACTTTCGCAACATCAACAACGAGTACAAGATTATCAGCTGTATGCAGAATAAT
AGCGGTTGGGAAGTTAGCCTGAATTTCAGCAATATGAACAGCAAAATCATTTGGACCCTGCAGGATACCGAAGGT
ATCAAAAAAACCGTTGTGTTTCAGTACACCCAGAACATTAACATCAGCGATTACATTAACCGCTGGATCTTTGTG
ACCATTACCAATAATCGTCTGAGCAACAGCAAGATCTATATTAACGGTCGCCTGATTAACGAAGAGAGCATTAGC
GATCTGGGTAATATTCATGCCAGCAACAACATCATGTTTAAACTGGATGGTTGTCGTGATCCGCATCGTTATATT
TGGATCAAATACTTCAACCTGTTTGATAAAGAACTGAACAAAAAAGAAATCAAAGACCTGTATGATAACCAGAGC
AATAGCGGCATTCTGAAAGATTTTTGGGGTGATTATCTGCAGTATGACAAACCGTATTACATGCTGAATCTGTAC
GATCCGAACAAATATCTGGATGTGAATAATGTGGGTATCCGTGGCTATATGTATCTGAAAGGTCCGCGTGGTCGT
ATTGTTACCACCAACATTTATCTGAATAGCACCCTGTATATGGGCACCAAATTCATCATTAAAAAGTATGCCAGC
GGCAACAAAGATAACATTGTGCGTAATAATGATCGCGTGTATATCAATGTGGTGGTGAAGAATAAAGAATATCGT
CTGGCCACCAATGCAAGCCAGGCAGGCGTTGAAAAAATTCTGAGCGCAGTTGAAATTCCGGATGTTGGTAATCTG
AGCCAGGTTGTTGTTATGAAAAGCGAAAATGATCAGGGCATTCGCAACAAATGCAAAATGAATCTGCAGGACAAT
AACGGCAACGATATTGGTTTTATTGGCTTCCACCAGTTCAACAACATTGCAAAACTGGTGGCGAGCAATTGGTAT
AATCGTCAGATTGGTAAAGCAAGCCGTACCTTTGGTTGTAGCTGGGAATTTATTCCGGTTGATGATGGTTGGGGT
GAAAGCAGCCTGGAAAATCTGTATTTTCAGGGTGCAAGTCATCATCACCACCATCACCATCATTAA
Polypeptide Sequence of rH_{C}/FA (His-tagged)
SEQ ID NO: 30
MLKYNCILNIKYEMDRDKLVDSSGYRSRINIGTGVKFSEIDKNQVQLSNLESSKIEVILNNGVIYNSMYENESTS
FWIRIPKYFRNINNEYKIISCMQNNSGWEVSLNFSNMNSKIIWTLQDTEGIKKTVVFQYTQNINISDYINRWIFV
TITNNRLSNSKIYINGRLINEESISDLGNIHASNNIMFKLDGCRDPHRYIWIKYFNLFDKELNKKEIKDLYDNQS
NSGILKDFWGDYLQYDKPYYMLNLYDPNKYLDVNNVGIRGYMYLKGPRGRIVTTNIYLNSTLYMGTKFIIKKYAS
GNKDNIVRNNDRVYINVVVKNKEYRLATNASQAGVEKILSAVEIPDVGNLSQVVVMKSENDQGIRNKCKMNLQDN
NGNDIGFIGFHQFNNIAKLVASNWYNRQIGKASRTFGCSWEFIPVDDGWGESSLENLYFQGASHHHHHHHH
Nucleotide Sequence of rLC/FA (His-tagged)
SEQ ID NO: 31
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CATGAACTGATTCATGTTCTGCATGGTCTGTATGGCACCTATATTACCGAATACAAAATTACCCCGAATGTGGTG
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCGAAAATCTGTATTTTCAGGGTGCCAGTCATCATCACCACCATCACCATCACTGA
Polypeptide Sequence of rLC/FA (His-tagged)
SEQ ID NO: 32
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HELIHVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGFNIGALSINYQGQNIGSDINSIKKLQGQGVVSRVVRLCSNSENLYFQGASHHHHHHHH
Nucleotide Sequence of rBoNT/F(0) (His-tagged)
SEQ ID NO: 33
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACACCAGCTGATTTATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGTGATTCCGCGTAAAGGCACCAAAGCACCGCCTCGTCTGTGTATTCGTGTGAATAAT
CGTGAACTGTTTTTTGTTGCAAGCGAGAGCAGCTATAACGAGAATGATATTAACACCCCGAAAGAGATTGACGAT
ACCACCAATCTGAATAACAACTATCGCAACAATCTGGATGAAGTGATCCTGGATTATAACAGCGAAACCATTCCG
CAGATTAGCAATCAGACCCTGAATACCCTGGTTCAGGATGATAGCTATGTTCCGCGTTATGATAGCAATGGCACC
AGCGAAATTGAAGAACATAATGTGGTTGATCTGAACGTGTTCTTTTATCTGCATGCACAGAAAGTGCCGGAAGGT
GAAACCAATATTAGCCTGACCAGCAGCATTGATACCGCACTGAGCGAAGAAAGCCAGGTTTATACCTTTTTTAGC
AGCGAATTCATCAACACCATTAACAAACCGGTTCATGCAGCACTGTTTATTAGCTGGATTAATCAGGTGATTCGC
GATTTTACCACCGAAGCAACCCAGAAAAGCACCTTTGATAAAATTGCCGATATTAGTCTGGTGGTGCCGTATGTT
GGTCTGGCACTGAATATTGGTAATGAAGTGCAGAAAGAGAACTTTAAAGAAGCCTTCGAACTGTTAGGTGCCGGT
ATTCTGCTGGAATTTGTGCCGGAACTGCTGATTCCGACCATTCTGGTTTTTACCATTAAGAGCTTTATTGGCAGC
AGCGAGAACAAGAACAAAATCATTAAAGCCATCAACAACAGCCTGATGGAACGCGAAACCAAATGGAAAGAAATT
TACAGCTGGATTGTGAGCAATTGGCTGACCCGTATCAATACCCAGTTTAACAAACGCAAAGAACAAATGTATCAG
GCCCTGCAGAATCAGGTTGATGCAATTAAAACCGTGATCGAATACAAATACAACAACTATACCAGCGACGAACGT
AATCGCCTGGAAAGCGAATACAACATTAATAACATTCGCGAAGAACTGAACAAAAAAGTGAGCCTGGCAATGGAA
AACATCGAACGTTTTATTACCGAAAGCAGCATCTTCTACCTGATGAAACTGATTAACGAAGCCAAAGTTAGCAAA
CTGCGCGAATATGATGAAGGCGTTAAAGAATATCTGCTGGACTATATTAGCGAACATCGTAGCATTCTGGGTAAT
AGCGTTCAAGAGCTGAATGATCTGGTTACCAGCACACTGAATAATAGCATTCCGTTTGAACTGAGCAGCTACACC
AACGATAAAATCCTGATCCTGTACTTCAACAAACTGTACAAGAAGATCAAGGACAACAGCATACTGGATATGCGC
TATGAAAACAACAAGTTCATTGATATCAGCGGCTATGGTAGCAACATTAGCATTAATGGTGATGTGTATATCTAC
AGCACCAACCGCAATCAGTTTGGTATTTATAGCAGCAAACCGAGCGAAGTTAATATTGCGCAGAATAACGATATC
ATCTACAACGGTCGCTATCAGAACTTTAGCATTAGCTTTTGGGTTCGCATTCCGAAATACTTTAACAAGGTGAAC
CTGAACAACGAGTACACCATTATTGATTGCATTCGCAATAATAACAGCGGCTGGAAAATCAGCCTGAACTATAAC
AAAATTATCTGGACCCTGCAGGATACCGCAGGTAATAATCAGAAACTGGTGTTTAACTACACCCAGATGATTAGC
ATCAGCGACTATATCAACAAATGGATCTTTGTGACCATTACCAACAATCGTCTGGGTAACAGCCGCATTTATATC
AATGGCAATCTGATCGACGAAAAAAGCATTTCAAATCTGGGCGATATTCACGTGAGCGATAACATTCTGTTCAAA
ATTGTTGGCTGCAACGATACCCGTTATGTTGGTATTCGTTACTTCAAAGTGTTTGATACGGAACTGGGCAAAACG
GAAATTGAAACCCTGTATAGTGATGAACCGGATCCGAGCATTCTGAAAGATTTTTGGGGTAATTATCTGCTGTAC
AACAAACGCTACTATCTGCTGAACCTGCTGCGTACCGATAAAAGCATTACACAGAATAGCAACTTTCTGAACATC
AATCAGCAGCGTGGTGTTTATCAGAAACCGAACATTTTTAGCAACACCCGTCTGTATACCGGTGTGGAAGTTATT
ATTCGTAAAAACGGTAGCACCGATATCAGCAACACCGATAACTTTGTGCGTAAAAATGACCTGGCCTATATTAAC
GTTGTTGATCGTGATGTTGAGTATCGTCTGTATGCGGATATTAGCATTGCCAAACCGGAAAAGATTATCAAACTG
ATCCGTACCAGCAACAGCAATAATTCACTGGGTCAGATTATCGTGATGGACAGCATTGGTAACAATTGCACCATG
AATTTCCAGAACAATAACGGTGGTAATATTGGCCTGCTGGGCTTTCATAGCAATAATCTGGTTGCAAGCAGCTGG
TATTACAACAACATCCGTAAAAATACCAGCAGTAATGGTTGCTTTTGGAGCTTTATCAGTAAAGAACATGGCTGG
CAAGAAAACGAGAACCTGTATTTTCAGGGTGCAAGTCATCATCACCATCACCACCATCATTAA
Polypeptide Sequence of rBoNT/F(0) (His-tagged)
SEQ ID NO: 34
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHQLIYALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGEL
KVPNLLDDDIYTVSEGENIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTEDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDCIRNNNSGWKISLNYN
KIIWTLQDTAGNNQKLVENYTQMISISDYINKWIFVTITNNRLGNSRIYINGNLIDEKSISNLGDIHVSDNILFK
IVGCNDTRYVGIRYFKVFDTELGKTEIETLYSDEPDPSILKDFWGNYLLYNKRYYLLNLLRTDKSITQNSNFLNI
NQQRGVYQKPNIFSNTRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTSSNGCFWSFISKEHGW
QENENLYFQGASHHHHHHHH
Nucleotide Sequence of rL_{H}N/F (His-tagged)
SEQ ID NO: 35
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACATGAACTGATTCATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGTGATTCCGCGTAAAGGCACCAAAGCACCGCCTCGTCTGTGTATTCGTGTGAATAAT
CGTGAACTGTTTTTTGTTGCAAGCGAGAGCAGCTATAACGAGAATGATATTAACACCCCGAAAGAGATTGACGAT
ACCACCAATCTGAATAACAACTATCGCAACAATCTGGATGAAGTGATCCTGGATTATAACAGCGAAACCATTCCG
CAGATTAGCAATCAGACCCTGAATACCCTGGTTCAGGATGATAGCTATGTTCCGCGTTATGATAGCAATGGCACC
AGCGAAATTGAAGAACATAATGTGGTTGATCTGAACGTGTTCTTTTATCTGCATGCACAGAAAGTGCCGGAAGGT
GAAACCAATATTAGCCTGACCAGCAGCATTGATACCGCACTGAGCGAAGAAAGCCAGGTTTATACCTTTTTTAGC
AGCGAATTCATCAACACCATTAACAAACCGGTTCATGCAGCACTGTTTATTAGCTGGATTAATCAGGTGATTCGC
GATTTTACCACCGAAGCAACCCAGAAAAGCACCTTTGATAAAATTGCCGATATTAGTCTGGTGGTGCCGTATGTT
GGTCTGGCACTGAATATTGGTAATGAAGTGCAGAAAGAGAACTTTAAAGAAGCCTTCGAACTGTTAGGTGCCGGT
ATTCTGCTGGAATTTGTGCCGGAACTGCTGATTCCGACCATTCTGGTTTTTACCATTAAGAGCTTTATTGGCAGC
AGCGAGAACAAGAACAAAATCATTAAAGCCATCAACAACAGCCTGATGGAACGCGAAACCAAATGGAAAGAAATT
TACAGCTGGATTGTGAGCAATTGGCTGACCCGTATCAATACCCAGTTTAACAAACGCAAAGAACAAATGTATCAG
GCCCTGCAGAATCAGGTTGATGCAATTAAAACCGTGATCGAATACAAATACAACAACTATACCAGCGACGAACGT
AATCGCCTGGAAAGCGAATACAACATTAATAACATTCGCGAAGAACTGAACAAAAAAGTGAGCCTGGCAATGGAA
AACATCGAACGTTTTATTACCGAAAGCAGCATCTTCTACCTGATGAAACTGATTAACGAAGCCAAAGTTAGCAAA
CTGCGCGAATATGATGAAGGCGTTAAAGAATATCTGCTGGACTATATTAGCGAACATCGTAGCATTCTGGGTAAT
AGCGTTCAAGAGCTGAATGATCTGGTTACCAGCACACTGAATAATAGCATTCCGTTTGAACTGAGCAGCTACACC
AACGATAAAATCCTGATCCTGTACTTCAACAAACTGTACAAGAAAGAAAACCTGTATTTTCAGGGTGCAAGCCAT
CATCACCACCATCACCATCATTAA
Polypeptide Sequence of rL_{H}N/F (His-tagged)
SEQ ID NO: 36
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHELIHALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGENIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DETTEATQKSTFDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYFNKLYKKENLYFQGASHHHHHHHH
Nucleotide Sequence of rH_{C}/F (His-tagged)
SEQ ID NO: 37
ATGATCAAGGATAACAGCATTCTGGATATGCGCTATGAGAACAACAAATTCATTGATATTAGCGGCTATGGCAGC
AACATTAGCATTAATGGTGATGTGTATATCTACAGCACCAACCGTAATCAGTTTGGCATTTATAGCAGCAAACCG
AGCGAAGTTAATATTGCCCAGAACAACGATATCATCTATAACGGTCGCTATCAGAACTTCAGCATTAGCTTTTGG
GTTCGCATTCCGAAATACTTCAATAAGGTGAACCTGAACAACGAGTATACCATCATTGATTGCATTCGCAATAAT
AACAGCGGCTGGAAAATTAGCCTGAACTACAACAAAATTATCTGGACCCTGCAGGATACCGCAGGTAATAATCAG
AAACTGGTGTTTAACTACACCCAGATGATTAGCATCAGCGACTATATCAACAAATGGATCTTTGTGACCATTACC
AATAATCGCCTGGGTAATAGCCGCATTTATATCAATGGTAACCTGATCGATGAGAAAAGCATTAGCAATCTGGGT
GATATTCATGTGAGCGATAACATCCTGTTTAAAATCGTGGGTTGTAACGATACCCGTTATGTTGGTATTCGCTAC
TTCAAAGTGTTTGATACCGAACTGGGTAAAACCGAAATTGAAACCCTGTATAGTGATGAACCGGATCCGAGCATT
CTGAAAGATTTTTGGGGTAATTATCTGCTGTACAACAAACGCTACTATCTGCTGAATCTGCTGCGTACCGATAAA
TCAATTACCCAGAATAGCAACTTCCTGAACATTAATCAGCAGCGTGGTGTTTATCAGAAACCGAACATTTTTAGC
AACACCCGTCTGTATACCGGTGTGGAAGTTATTATTCGTAAAAATGGCAGCACCGATATCAGCAACACCGATAAC
TTTGTTCGCAAAAATGATCTGGCGTATATCAACGTTGTTGATCGTGATGTTGAATATCGTCTGTATGCCGATATT
AGCATTGCCAAACCGGAAAAAATCATCAAACTGATCCGTACCAGCAACAGCAATAATTCACTGGGTCAGATTATT
GTGATGGATAGCATTGGTAATAACTGCACCATGAACTTTCAGAACAATAACGGTGGTAATATTGGTCTGCTGGGC
TTTCATAGTAATAATCTGGTTGCAAGCAGCTGGTATTATAACAACATCCGTAAAAATACCAGCAGCAATGGTTGC
TTTTGGAGCTTTATTAGCAAAGAACATGGCTGGCAAGAAAACGAGAATCTGTATTTTCAGGGTGCAAGTCATCAT
CACCACCATCACCATCATTAA
Polypeptide Sequence of rH_{C}/F (His-tagged)
SEQ ID NO: 38
MIKDNSILDMRYENNKFIDISGYGSNISINGDVYIYSTNRNQFGIYSSKPSEVNIAQNNDIIYNGRYQNFSISFW
VRIPKYFNKVNLNNEYTIIDCIRNNNSGWKISLNYNKIIWTLQDTAGNNQKLVFNYTQMISISDYINKWIFVTIT
NNRLGNSRIYINGNLIDEKSISNLGDIHVSDNILFKIVGCNDTRYVGIRYFKVEDTELGKTEIETLYSDEPDPSI
LKDFWGNYLLYNKRYYLLNLLRTDKSITQNSNFLNINQQRGVYQKPNIFSNTRLYTGVEVIIRKNGSTDISNTDN
FVRKNDLAYINVVDRDVEYRLYADISIAKPEKIIKLIRTSNSNNSLGQIIVMDSIGNNCTMNFQNNNGGNIGLLG
FHSNNLVASSWYYNNIRKNTSSNGCFWSFISKEHGWQENENLYFQGASHHHHHHHH
Nucleotide Sequence of rLC/F (His-tagged)
SEQ ID NO: 39
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACATGAACTGATTCATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGAGAACCTGTATTTTCAGGGTGCAAGTCATCATCACCATCACCACCATCATTAA
Polypeptide Sequence of rLC/F (His-tagged)
SEQ ID NO: 40
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHELIHALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGEL
KVPNLLDDDIYTVSEGENIGNLAVNNRGQNIKLNPKIIDSIPDKGLVEKIVKFCKSENLYFQGASHHHHHHHH
Nucleotide Sequence of Cationic rH_{C}/A (His-tagged)
SEQ ID NO: 41
ATGATCATCAACACCAGCATTCTGAACCTGCGTTATGAAAGCAAACATCTGATTGATCTGAGCCGTTATGCCAGC
AAAATCAATATAGGCAGCAAGGTTAACTTCGACCCGATTGACAAAAATCAGATACAGCTGTTTAATCTGGAAAGC
AGCAAAATTGAGGTGATCCTGAAAAAAGCGATCGTGTATAATAGCATGTACGAGAATTTTTCGACCAGCTTTTGG
ATTCGCATCCCGAAATACTTTAACAAGATTAGCCTGAACAACGAGTATACCATCATTAACTGCATGGAAAACAAT
AGCGGTTGGAAAGTCAGCCTGAATTATGGCGAAATTATCTGGACCCTGCAGGATACCAAAGAAATCAAACAGCGT
GTGGTGTTCAAATACAGCCAGATGATTAATATCAGCGACTATATCAACCGCTGGATTTTTGTGACCATTACCAAT
AATCGGCTGAACAAGAGCAAGATCTATATTAACGGTCGTCTGATTGACCAGAAACCGATTAGTAATCTGGGTAAT
ATTCATGCGAGCAACAAAATCATGTTTAAACTGGATGGTTGCCGTGATACCCATCGTTATATTTGGATCAAATAC
TTCAACCTGTTCGATAAAGAGTTGAACGAAAAAGAAATTAAAGACCTGTACGATAACCAGAGCAATAGCGGCATA
CTGAAAGATTTTTGGGGAGATTATCTGCAGTATGACAAACCGTATTATATGCTGAATCTGTACGACCCGAATAAA
TACGTGGATGTTAATAATGTGGGCATCCGTGGTTATATGTACCTGAAAGGTCCGCGTGGTAGCGTTATGACCACA
AACATTTATCTGAATAGCAGCCTGTATCGCGGAACCAAATTCATCATTAAAAAGTATGCCAGCGGCAACAAGGAT
AATATTGTGCGTAATAATGATCGCGTGTACATTAACGTTGTGGTGAAGAATAAAGAATATCGCCTGGCAACCAAT
GCAAGCCAGGCAGGCGTTGAAAAAATTCTGAGTGCCCTGGAAATTCCGGATGTTGGTAATCTGAGCCAGGTTGTT
GTGATGAAAAGCAAAAACGATAAAGGCATCACCAACAAATGCAAGATGAATCTGCAGGACAATAACGGCAATGAT
ATTGGCTTCATTGGCTTTCACCAGTTTAACAACATTGCAAAACTGGTTGCGAGCAATTGGTATAATCGTCAGATT
GAACGTAGCAGTCGTACCCTGGGTTGTAGCTGGGAATTTATCCCTGTGGATGATGGTTGGGGTGAACGTCCGCTG
AAGCTTGCGGCCGCACTCGAGCACCACCACCACCACCACTGA
Polypeptide Sequence of Cationic rH_{C}/A (His-tagged)
SEQ ID NO: 42
MIINTSILNLRYESKHLIDLSRYASKINIGSKVNFDPIDKNQIQLFNLESSKIEVILKKAIVYNSMYENFSTSFW
IRIPKYFNKISLNNEYTIINCMENNSGWKVSLNYGEIIWTLQDTKEIKQRVVFKYSQMINISDYINRWIFVTITN
NRLNKSKIYINGRLIDQKPISNLGNIHASNKIMFKLDGCRDTHRYIWIKYENLEDKELNEKEIKDLYDNQSNSGI
LKDFWGDYLQYDKPYYMLNLYDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDKGITNKCKMNLQDNNGND
IGFIGFHQFNNIAKLVASNWYNRQIERSSRTLGCSWEFIPVDDGWGERPLKLAAALEHHHHHH
Nucleotide Sequence of rH_{C}/AB (His-tagged)
SEQ ID NO: 43
ATGATTCTGAACAATATTATCCTGAACCTGCGTTACAAAGACAACAATCTGATCGATCTGAGCGGCTATGGTGCA
AAAGTTGAAGTCTACGACGGTGTCGAACTGAACGATAAAAACCAGTTCAAACTGACCTCATCGGCTAACTCAAAA
ATTCGTGTGACGCAGAACCAAAACATCATCTTCAACTCGGTCTTTCTGGACTTCAGCGTGTCTTTCTGGATTCGC
ATCCCGAAATATAAAAATGATGGCATCCAGAACTACATCCATAACGAATACACCATCATCAACTGTATGAAAAAC
AACAGTGGTTGGAAAATTTCCATCCGTGGCAACCGCATTATCTGGACCCTGATTGATATCAATGGTAAAACGAAA
AGCGTGTTTTTCGAATACAACATCCGTGAAGATATCTCTGAATACATCAATCGCTGGTTTTTCGTGACCATTACG
AACAATCTGAACAATGCGAAAATCTATATCAACGGCAAACTGGAAAGTAATACCGACATCAAAGATATTCGTGAA
GTTATCGCCAACGGTGAAATCATCTTCAAACTGGATGGCGACATCGATCGCACCCAGTTCATTTGGATGAAATAC
TTCTCCATCTTCAACACGGAACTGAGTCAGTCCAATATCGAAGAACGCTACAAAATCCAATCATACTCGGAATAC
CTGAAAGATTTCTGGGGTAACCCGCTGATGTACAACAAAGAATACTACATGTTCAACGCGGGCAACAAAAACTCA
TACATCAAACTGAAAAAAGATTCGCCGGTGGGTGAAATCCTGACCCGTAGCAAATACAACCAGAACTCTAAATAC
ATCAACTATCGCGATCTGTACATTGGCGAAAAATTTATTATCCGTCGCAAAAGCAACTCTCAGAGTATTAATGAT
GACATCGTGCGTAAAGAAGACTACATCTATCTGGATTTCTTTAATCTGAACCAAGAATGGCGCGTTTATACCTAC
AAATACTTCAAAAAAGAAGAAATGAAACTGTTCCTGGCCCCGATTTACGACAGCGATGAATTTTACAACACCATC
CAGATCAAAGAATACGATGAACAGCCGACGTATAGTTGCCAACTGCTGTTCAAAAAAGACGAAGAATCCACCGAT
GAAATTGGCCTGATTGGTATCCACCGTTTCTATGAAAGCGGTATCGTTTTCGAAGAATACAAAGATTACTTCTGT
ATCTCTAAATGGTATCTGAAAGAAGTCAAACGCAAACCGTACAACCTGAAACTGGGCTGCAACTGGCAATTTATC
CCGAAAGACGAAGGCTGGACCGAAAAGCTTGCGGCCGCACTCGAGCACCACCACCACCACCACTGA
Polypeptide Sequence of rH_{C}/AB (His-tagged)
SEQ ID NO: 44
MILNNIILNLRYKDNNLIDLSGYGAKVEVYDGVELNDKNQFKLTSSANSKIRVTQNQNIIFNSVFLDFSVSFWIR
IPKYKNDGIQNYIHNEYTIINCMKNNSGWKISIRGNRIIWTLIDINGKTKSVFFEYNIREDISEYINRWFFVTIT
NNLNNAKIYINGKLESNTDIKDIREVIANGEIIFKLDGDIDRTQFIWMKYFSIFNTELSQSNIEERYKIQSYSEY
LKDFWGNPLMYNKEYYMFNAGNKNSYIKLKKDSPVGEILTRSKYNQNSKYINYRDLYIGEKFIIRRKSNSQSIND
DIVRKEDYIYLDFFNLNQEWRVYTYKYFKKEEMKLFLAPIYDSDEFYNTIQIKEYDEQPTYSCQLLFKKDEESTD
EIGLIGIHRFYESGIVFEEYKDYFCISKWYLKEVKRKPYNLKLGCNWQFIPKDEGWTEKLAAALEHHHHHH
Nucleotide Sequence of rH_{C}/A Variant Y1117V H1253K (His-tagged)
SEQ ID NO: 45
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTTCaAaCAGTTCAACAATATTGCTAAACTGGTAGCGAGCAATTGGTACAATCGTCAGATT
GAGCGCAGCAGCCGTACTTTGGGCTGTAGCTGGGAGTTTATCCCGGTCGATGATGGTTGGGGCGAACGTCCGCTG
CACCATCACCATCACCATCACCATCACCATT
Polypeptide Sequence of rH_{C}/A Variant Y1117V H1253K (His-tagged)
SEQ ID NO: 46
MIINTSILNLRYESNHLIDLSRYASKINIGSKVNFDPIDKNQIQLENLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGFKQFNNIAKLVASNWYNRQIERSSRTLGCSWEFIPVDDGWGERPLHHHHHHHHHH
Nucleotide Sequence of rH_{C}/A Variant Y1117V F1252Y H1253K L1278F
(His-tagged)
SEQ ID NO: 47
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTaCaAaCAGTTCAACAATATTGCTAAACTGGTAGCGAGCAATTGGTACAATCGTCAGATT
GAGCGCAGCAGCCGTACTTTtGGCTGTAGCTGGGAGTTTATCCCGGTCGATGATGGTTGGGGCGAACGTCCGCTG
CACCATCACCATCACCATCACCATCACCATTAA
Polypeptide Sequence of rH_{C}/A Variant Y1117V F1252Y H1253K L1278F
(His-tagged)
SEQ ID NO: 48
MIINTSILNLRYESNHLIDLSRYASKINIGSKVNFDPIDKNQIQLENLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGYKQFNNIAKLVASNWYNRQIERSSRTFGCSWEFIPVDDGWGERPLHHHHHHHHHH
Nucleotide Sequence of rH_{C}/A Variant Y1117V F1252Y H1253K L1278H
(His-tagged)
SEQ ID NO: 49
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTaCaAaCAGTTCAACAATATTGCTAAACTGGTAGCGAGCAATTGGTACAATCGTCAGATT
GAGCGCAGCAGCCGTACTcatGGCTGTAGCTGGGAGTTTATCCCGGTCGATGATGGTTGGGGCGAACGTCCGCTG
CACCATCACCATCACCAT
Polypeptide Sequence of rH_{C}/A Variant Y1117V F1252Y H1253K L1278H
(His-tagged)
SEQ ID NO: 50
MIINTSILNLRYESNHLIDLSRYASKINIGSKVNFDPIDKNQIQLENLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGYKQFNNIAKLVASNWYNRQIERSSRTHGCSWEFIPVDDGWGERPLHHHHHH
Polypeptide Sequence of BoNT/A-UniProt P10845
SEQ ID NO: 51
MPFVNKQFNYKDPVNGVDIAYIKIPNVGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRESPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKEDKLYKMLTEIYTEDNFVKFFKV
LNRKTYLNFDKAVFKINIVPKVNYTIYDGENLRNTNLAANFNGQNTEINNMNFTKLKNFT
GLFEFYKLLCVRGIITSKTKSLDKGYNKALNDLCIKVNNWDLFFSPSEDNETNDLNKGEE
ITSDTNIEAAEENISLDLIQQYYLTENEDNEPENISIENLSSDIIGQLELMPNIEREPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIESG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDEDASLKDALLKYIYDNRGTLIGQVDRLKDK
VNNTLSTDIPFQLSKYVDNQRLLSTFTEYIKNIINTSILNLRYESNHLIDLSRYASKINI
GSKVNFDPIDKNQIQLENLESSKIEVILKNAIVYNSMYENFSTSFWIRIPKYENSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYENLEDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQENNIAK
LVASNWYNRQIERSSRTLGCSWEFIPVDDGWGERPL
Polypeptide Sequence of BoNT/B-UniProt P10844
SEQ ID NO: 52
MPVTINNENYNDPIDNNNIIMMEPPFARGTGRYYKAFKITDRIWIIPERYTFGYKPEDEN
KSSGIFNRDVCEYYDPDYLNTNDKKNIFLQTMIKLENRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVENNVQENKGASIFNRRGYFSDPALILMHELIHVLHGLY
GIKVDDLPIVPNEKKFFMQSTDAIQAEELYTFGGQDPSIITPSTDKSIYDKVLQNERGIV
DRLNKVLVCISDPNININIYKNKFKDKYKFVEDSEGKYSIDVESEDKLYKSLMFGFTETN
IAENYKIKTRASYFSDSLPPVKIKNLLDNEIYTIEEGENISDKDMEKEYRGQNKAINKQA
YEEISKEHLAVYKIQMCKSVKAPGICIDVDNEDLFFIADKNSESDDLSKNERIEYNTQSN
YIENDFPINELILDTDLISKIELPSENTESLTDFNVDVPVYEKQPAIKKIFTDENTIFQY
LYSQTFPLDIRDISLTSSEDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDENDINSKLNEGINQAIDNINNFINGCSV
SYLMKKMIPLAVEKLLDEDNTLKKNLLNYIDENKLYLIGSAEYEKSKVNKYLKTIMPEDL
SIYTNDTILIEMENKYNSEILNNIILNLRYKDNNLIDLSGYGAKVEVYDGVELNDKNQFK
LTSSANSKIRVTQNQNIIFNSVELDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIENTELSQSNIEERYKIQSY
SEYLKDFWGNPLMYNKEYYMENAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
Polypeptide Sequence of BoNT/C-UniProt P18640
SEQ ID NO: 53
MPITINNENYSDPVDNKNILYLDTHLNTLANEPEKAFRITGNIWVIPDRFSRNSNPNLNK
PPRVTSPKSGYYDPNYLSTDSDKDPFLKEIIKLFKRINSREIGEELIYRLSTDIPFPGNN
NTPINTFDFDVDENSVDVKTRQGNNWVKTGSINPSVIITGPRENIIDPETSTEKLINNTE
AAQEGFGALSIISISPREMLTYSNATNDVGEGRFSKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSENKYIGEYKQKLIRKYRFVVESSGEVTVNRNKEVELYNELTQIFTE
FNYAKIYNVQNRKIYLSNVYTPVTANILDDNVYDIQNGFNIPKSNLNVLFMGQNLSRNPA
LRKVNPENMLYLFTKFCHKAIDGRSLYNKTLDCRELLVKNTDLPFIGDISDVKTDIFLRK
DINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEFDRNTKAKLINLIDSHNIILVGEVDKLKAKVNNSF
QNTIPFNIFSYTNNSLLKDIINEYENNINDSKILSLQNRKNTLVDTSGYNAEVSEEGDVQ
LNPIFPFDFKLGSSGEDRGKVIVTQNENIVYNSMYESFSISEWIRINKWVSNLPGYTIID
SVKNNSGWSIGIISNFLVFTLKQNEDSEQSINESYDISNNAPGYNKWFFVTVTNNMMGNM
KIYINGKLIDTIKVKELTGINFSKTITFEINKIPDTGLITSDSDNINMWIRDFYIFAKEL
DGKDINILENSLQYTNVVKDYWGNDLRYNKEYYMVNIDYLNRYMYANSRQIVENTRRNNN
DENEGYKIIIKRIRGNTNDTRVRGGDILYFDMTINNKAYNLFMKNETMYADNHSTEDIYA
IGLREQTKDINDNIIFQIQPMNNTYYYASQIFKSNENGENISGICSIGTYRFRLGGDWYR
HNYLVPTVKQGNYASLLESTSTHWGFVPVSE
Polypeptide Sequence of BoNT/D-UniProt P19321
SEQ ID NO: 54
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPEMGDS
STPEDTFDFTRHTTNIAVEKFENGSWKVTNIITPSVLIFGPLPNILDYTASLTLQGQQSN
PSFEGFGTLSILKVAPEFLLTESDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNFDKDNTGNFVVNIDKENSLYSDLTNVMSE
VVYSSQYNVKNRTHYFSRHYLPVFANILDDNIYTIRDGENLINKGENIENSGQNIERNPA
LQKLSSESVVDLFTKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLELNWANE
VVEDETTNIMKKDTLDKISDVSVIIPYIGPALNIGNSALRGNENQAFATAGVAFLLEGEP
EFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIR
ECSVTYLFKNMLPKVIDELNKEDLRTKTELINLIDSHNIILVGEVDRLKAKVNESFENTM
PFNIFSYTNNSLLKDIINEYENSINDSKILSLQNKKNALVDTSGYNAEVRVGDNVQLNTI
YTNDFKLSSSGDKIIVNLNNNILYSAIYENSSVSFWIKISKDLINSHNEYTIINSIEQNS
GWKLCIRNGNIEWILQDVNRKYKSLIFDYSESLSHTGYTNKWFFVTITNNIMGYMKLYIN
GELKQSQKIEDLDEVKLDKTIVFGIDENIDENQMLWIRDENIFSKELSNEDINIVYEGQI
LRNVIKDYWGNPLKEDTEYYIINDNYIDRYIAPESNVLVLVQYPDRSKLYTGNPITIKSV
SDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRFSFKNAYTPVAVTNYETKLLS
TSSFWKFISRDPGWVE
Polypeptide Sequence of BoNT/E-UniProt Q00496
SEQ ID NO: 55
MPKINSFNYNDPVNDRTILYIKPGGCQEFYKSENIMKNIWIIPERNVIGTTPQDFHPPTS
LKNGDSSYYDPNYLQSDEEKDRFLKIVTKIFNRINNNLSGGILLEELSKANPYLGNDNTP
DNQFHIGDASAVEIKFSNGSQDILLPNVIIMGAEPDLFETNSSNISLRNNYMPSNHRFGS
IAIVTESPEYSFRENDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLRTKFQVKCRQTYIGQYKYFKL
SNLLNDSIYNISEGYNINNLKVNFRGQNANLNPRIITPITGRGLVKKIIRFCKNIVSVKG
IRKSICIEINNGELFFVASENSYNDDNINTPKEIDDTVTSNNNYENDLDQVILNENSESA
PGLSDEKLNLTIQNDAYIPKYDSNGTSDIEQHDVNELNVFFYLDAQKVPEGENNVNLTSS
IDTALLEQPKIYTFFSSEFINNVNKPVQAALFVSWIQQVLVDETTEANQKSTVDKIADIS
IVVPYIGLALNIGNEAQKGNEKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRELTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYENIFDKELDETEIQTLYSNEPNTNILKDEWGNYL
LYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRFNQVVVMNSVGNCTMNF
KNNNGNNIGLLGFKADTVVASTWYYTHMRDHTNSNGCFWNFISEEHGWQEK
Polypeptide Sequence of BoNT/F-UniProt A7GBG3
SEQ ID NO: 56
MPVVINSFNYNDPVNDDTILYMQIPYEEKSKKYYKAFEIMRNVWIIPERNTIGTDPSDED
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTENDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKF
KVKCRNTYFIKYGFLKVPNLLDDDIYTVSEGFNIGNLAVNNRGQNIKLNPKIIDSIPDKG
LVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYENKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDC
IRNNNSGWKISLNYNKIIWTLQDTAGNNQKLVFNYTQMISISDYINKWIFVTITNNRLGN
SRIYINGNLIDEKSISNLGDIHVSDNILFKIVGCNDTRYVGIRYFKVFDTELGKTEIETL
YSDEPDPSILKDFWGNYLLYNKRYYLLNLLRTDKSITQNSNFLNINQQRGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
Polypeptide Sequence of BoNT/G-UniProt Q60393
SEQ ID NO: 57
MPVNIKXFNYNDPINNDDIIMMEPENDPGPGTYYKAFRIIDRIWIVPERFTYGFQPDQEN
ASTGVFSKDVYEYYDPTYLKTDAEKDKFLKTMIKLFNRINSKPSGQRLLDMIVDAIPYLG
NASTPPDKFAANVANVSINKKIIQPGAEDQIKGLMTNLIIFGPGPVLSDNFTDSMIMNGH
SPISEGFGARMMIRFCPSCLNVENNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKFDKLYKALMFGFTETNL
AGEYGIKTRYSYFSEYLPPIKTEKLLDNTIYTQNEGENIASKNLKTEFNGQNKAVNKEAY
EEISLEHLVIYRIAMCKPVMYKNTGKSEQCIIVNNEDLFFIANKDSFSKDLAKAETIAYN
TQNNTIENNESIDQLILDNDLSSGIDLPNENTEPFTNEDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENFKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDENDIDFKLNQSINLAINNIDDFI
NQCSISYLMNRMIPLAVKKLKDFDDNLKRDLLEYIDTNELYLLDEVNILKSKVNRHLKDS
IPFDLSLYTKDTILIQVENNYISNISSNAILSLSYRGGRLIDSSGYGATMNVGSDVIEND
IGNGQFKLNNSENSNITAHQSKFVVYDSMFDNESINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDEKLINCTDTTKFVWIKDENIFGRELNATEVS
SLYWIQSSTNTLKDFWGNPLRYDTQYYLFNQGMQNIYIKYFSKASMGETAPRTNENNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
Polypeptide Sequence of TeNT-UniProt P04958
SEQ ID NO: 58
MPITINNFRYSDPVNNDTIIMMEPPYCKGLDIYYKAFKITDRIWIVPERYEFGTKPEDEN
PPSSLIEGASEYYDPNYLRTDSDKDRFLQTMVKLFNRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGFNIESKDLKSEYKGQNMRVNT
NAFRNVDGSGLVSKLIGLCKKIIPPTNIRENLYNRTASLTDLGGELCIKIKNEDLTFIAE
KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLQWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKQLLEFDTQSKNILMQYIKANSKFIG
ITELKKLESKINKVFSTPIPFSYSKNLDCWVDNEEDIDVILKKSTILNLDINNDIISDIS
GENSSVITYPDAQLVPGINGKAIHLVNNESSEVIVHKAMDIEYNDMENNFTVSFWLRVPK
VSASHLEQYGTNEYSIISSMKKHSLSIGSGWSVSLKGNNLIWTLKDSAGEVRQITERDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWIND
Polypeptide Sequence of BoNT/X
SEQ ID NO: 59
MKLEINKENYNDPIDGINVITMRPPRHSDKINKGKGPFKAFQVIKNIWIVPERYNETNNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNEDTGKIETSRQQNSLIFEELLTEGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGENISSQGSNDFQGQLLESSYFEKIESNALRAFI
KICPRNGLLYNAIYRNSKNYLNNIDLEDKKTTSKTNVSYPCSLLNGCIEVENKDLFLISN
KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDESDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKFMIKLSNSYLTKEMIPKVQDNLKNEDLETKKTLDK
FIKEKEDILGTNLSSSLRRKVSIRLNKNIAFDINDIPESEFDDLINQYKNEIEDYEVLNL
GAEDGKIKDLSGTTSDINIGSDIELADGRENKAIKIKGSENSTIKIAMNKYLRFSATDNE
SISFWIKHPKPTNLLNNGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYENSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKTNWYFIPKDEGWDED
Nucleotide Sequence of mrBoNT/A
SEQ ID NO: 60
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAAGCATCTGATTGATCTGAGCCGTTATGCTAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAAGGCCATTGTCTACAACTCCATGTACGAGAATTTCTCCACCAGCTTCTGGATTCGCATCCCG
AAATACTTCAACAAGATTAGCCTGAACAACGAGTATACTATCATCAACTGTATGGAGAACAACAGCGGTTGGAAG
GTGTCTCTGAACTATGGTGAGATCATTTGGACCTTGCAGGACACCAAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AAGAGCAAGATTTACATCAATGGTCGCTTGATCGATCAGAAACCGATTAGCAACCTGGGTAATATCCACGCAAGC
AACAAGATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACAAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
Polypeptide Sequence of mrBoNT/A
SEQ ID NO: 61
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNEDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKELNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLENLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLEDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQENNIAKLVASNWYNRQIERSS
RTLGCSWEFIPVDDGWGERPL
Polypeptide Sequence of Unmodified BoNT/A1
SEQ ID NO: 62
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYENSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
Polypeptide Sequence of mrBoNT/AB
SEQ ID NO: 63
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNEDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
Polypeptide Sequence of mrBoNT/AB(0)
SEQ ID NO: 64
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
Polypeptide Sequence of mrBoNT/A(0)
SEQ ID NO: 65
MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHQLIYAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGENLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIP
KYFNKISLNNEYTIINCMENNSGWKVSLNYGEIIWTLQDTKEIKQRVVFK
YSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYL
NSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFI
GFHQFNNIAKLVASNWYNRQIERSSRTLGCSWEFIPVDDGWGERPL

### EXAMPLES

Materials and Methods

Animals

Six wild-type (WT) mice were used in the pilot study; 24 CX3CR1-GFP mice (breeding pairs purchased from the JAX Laboratory, USA) were used for the main study; 12 mice were used in the imaging study. Mice were aged 3-5 months at the starting of imaging. For imaging experiments, mice were fitted with a cranial window with access port to allow for imaging and neurotoxin injection (more details below).

CX3CR1-GFP mice are described elsewhere, see Nimmerjahn et al. (Science. 2005 May 27; 308(5726):1314-8), incorporated herein by reference.

Botulinum Neurotoxin (BoNT) and Control Injection

BoNT/A was used, namely Dysport (abobotulinum toxin A (aboBoNT-A)), dissolved in 2 μL saline. In control experiments, Vehicle only (2 μL saline) solutions were used.

Intracortically-administered Dysport was well tolerated: 3 WT mice (pilot study) were administered with 0.5 U/animal of Dysport (dissolved in 2 μL of saline) and 3 WT mice with Vehicle solution and observed for clinical signs and measured their body weight for 2 days.

No clinical signs or behavioural changes associated with neurotoxic effects, and no significant difference in the weight between the treatment groups, thus the dose of 0.5 U/animal was approved for the main study.

For the main study, 0.5 U of Dysport (dissolved in 2 μL saline) or Vehicle (2 μL saline) solutions were injected (during the first imaging session) through a silicone access port (of the cranial window) into the somatosensory cortex of the mouse. Solution was injected at the rate of 2 nL/second.

Experimental Set-Up

Thirty days before Dysport injection (Day −30), the mice were fitted with a cranial window with access port (thirty days were allowed to elapse before further experimentation, to provide time for brain/neuroimmune response activity and behaviour to normalise following instalment of the cranial window). In the course of the study, animals were imaged at several timepoints. On Day 0 (Day of the cortical injection of either Dysport or Vehicle) imaging was done just before the laser injury as well at 6-, 18-, 30- and 42-minutes post-injury. In addition, imaging was done on Day 1 (24 hours post injection/23.5 hours post laser injury) and at Day 2 (48 hours post-injection/47.5 hours post laser injury). On the injection day (Day 0), baseline imaging (imaging session #1) was conducted immediately before injection. Imaging session #2 was conducted one day after injection (Day 1). Imaging session #3 was conducted two days after injection (Day 2).

Craniotomy and Instalment of the Glass Plates

For implantation of a cranial window with access port (the implantation replacing a piece of the skull to allow imaging of and access to the brain), mice were anesthetized with ketamine/xylazine. The cranial window was inserted over the somatosensory cortex at the following coordinates: AP −1.8, ML −2.0 from Bregma. Dental drill (HP4-917, Foredom) was used to remove a round-shaped (d=4 mm) piece of skull, and the hole in the bone was covered with a round cover glass (d=5 mm; #72296-05, Electron Microscopy Sciences). Before implantation of the cover glass, a small eccentrically located hole was drilled in the glass and filled with silicone (Sylgard 184) to create an access port for intracortical injections in chronic cranial windows. Headplate was placed over the cover glass and fixed with dental cement (Rapid Repair; Dentsply) mixed with cyanoacrylate glue (Loctite 401; Henkel) to a skull surface.

Image Acquisition Setup

Illumination: 900 nm, femtosecond laser Mai-Tai Broad Band DeepSee.

Emission filter: Channel 2—band pass 468-552 nm for the enhanced-Green Fluorescent Protein (EGFP) fluorescence.

Stacks of 60 images were collected with the vertical step of 2 μm (total imaged depth of stacks: 120 μm) in at least two imaging areas. Each imaging area covered 500×500 μm² of space in xy coordinates.

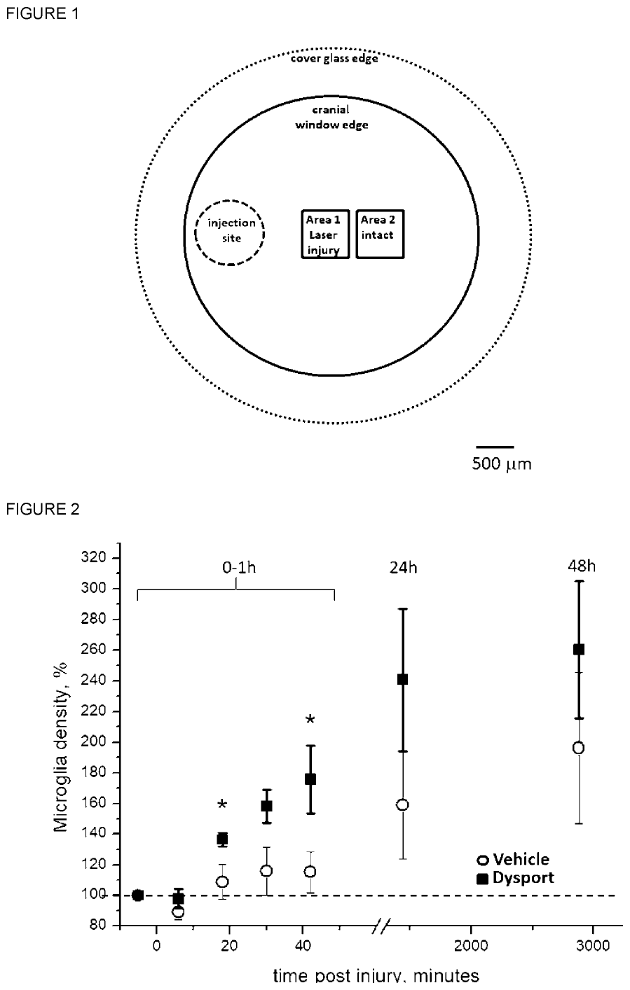

To induce laser injury in Area 1 (see FIG. 1 for schematic diagram of imaging areas' location), micrometre-size region of interest (ROI) was selected in the focal plane at the depth of 25-35 micrometres from the cortical surface and the infrared laser at 100% intensity was applied for seconds during the first hour after intracortical injection. In order to target light (laser) intensity at a particular region of the brain, to provide a distinct focal point of laser-induced injury, two-photon microscopy technology was employed. In rare instances of unsuccessful laser injury induction during the first imaging session, the injury induction was repeated 24 hours later.

Imaging Sessions

In the course of the study, animals were imaged at several timepoints. On Day 0 (Day of the cortical injection of either Dysport or Vehicle) imaging was done just before the laser injury as well at 6-, 18-, 30- and 42-minutes post-injury. In addition, imaging was done on Day 1 (24 hours post injection/23.5 hours post laser injury) and at Day 2 (48 hours post-injection/47.5 hours post laser injury).

Data Processing and Analysis

After acquisition, the image stacks were aligned in Fiji/ImageJ software. Shifts of tissue in x, y and z directions were compensated using plugins in the Fiji software. The aligned image stacks were used for analysis of density, morphology and laser-induced process extension of microglia cells using the set of plugins described below.

For analysis of microglia density and morphology changes “MorphoLibJ” was used: a collection of mathematical morphology methods and plugins for ImageJ, created at INRA-IJPB Modeling and Digital Imaging lab. The image stack with thickness of 40 micrometres was included in data analysis in Area 1 (±20 micrometres from the slice with laser injury location) and in Area 2 (the most superficial 40 micrometres).

The following sequence of steps was implemented in MorphoLibJ plugin:
1) Aligned image stacks were filtered using GreyScale and 3D Morphological filters to enhance the contrast between the cells and surrounding tissue;
2) Automatic Otsu thresholding was applied to all images to create a binary mask of the cells (minimal size threshold 20 pixels, no maximal threshold);
3) “Particle analysis 3D” and “Intensity measurements 3d” plugins were applied to the image stacks to get the numerical results.

Objects number (to calculate density of the cells), total volume and mean volume of the cells, sphericity index of the cells and mean fluorescent intensity of the cells were calculated. For the 3D analysis used in this study, the sphericity index can be defined as the ratio of the squared volume (V) over the cube of the surface area (S):

$$sphericity = 36 π \frac{{V}^{2}}{{S}^{3}}$$

For measurement of microglia process extension toward laser injury, two circles with diameter of 25 (small circle=s.c.) and 50 micrometres (big circle=b.c.) were drawn around the epicentre of the injury site and redistribution of fluorescence from the large circle into the smaller circle was measured. Microglia process extension was calculated using the following equation:

$$Microglia process extension = \frac{fluorescence s . c . ( tx ) - fluorescence s . c . ( t )}{fluorescence b . c . ( t 0 )} \\ where tx = timepoint x , and t 0 = timepoint 0 ( baseline )$$

Numerical data were then transferred to Microsoft Excel, Microcal Origin and Graphpad Prism software for statistical analysis and plotting of the graphs. For statistical comparisons between the treatment groups, either Student t-test was used (in case of normal data distribution) or Mann-Whitney non-parametric test (in case the data were distributed not normally). For statistical comparisons vs baseline, either paired Student t-test was used (in case of normal data distribution) or Wilcoxon signed ranks test (in case the data were distributed not normally). For test of normality of distribution, the Shapiro-Wilcoxon normality test was used.

### Example 1—Clostridial Neurotoxin Induces a Significant Increase in Microglia Density, Decrease in Soma Sphericity and Enhancement of Processes Extension

The inventors established a robust, controlled experimental system to investigate the effect of clostridial neurotoxin administration on the activity of the neuroimmune response subsequent to neural tissue damage. The system allows for induction of precise neural damage in the brain, such neural damage representing that caused by brain disorders described herein (e.g. such as neural tissue damage caused by the presence of a brain tumor, or an infection, or nerve death due to oxygen depletion following death).

The system employs transgenic mice in which key immune cells of the neuroimmune response (microglia) specifically express GFP, allowing for time lapse imaging of said immune cells such that their activity in response to neural damage, in either the presence or absence of clostridial neurotoxin administration, can be tracked and analysed. A further advantage of the system (employing advanced light microscopy) is that it can be coupled to a laser to induce the precise area of neuron damage. Full details of the system are provided in the materials and methods section (above).

Two imaging areas were imaged in all animals (see FIG. 1 for illustration): Area 1, closer to the injection site (distance range 600-1000 micrometres from the edge of the Area to the injection site), Area 2 (distance at least 1000 micrometres).

In all analysed mice, a clear expression pattern of GFP was observed consistent with the morphology of microglial cells. Laser injury was introduced in the Area 1 at Day 0, within one hour after intracortical injection of Dysport (see FIGS. 1 and 2). The chronic window with access port was confirmed as a reliable model for repetitive post-injection imaging in the CX3CR1-GFP mouse line. For optimal imaging, a superficial cortical layer was laser-injured and imaged. The laser injury was successfully induced in all 12 mice.

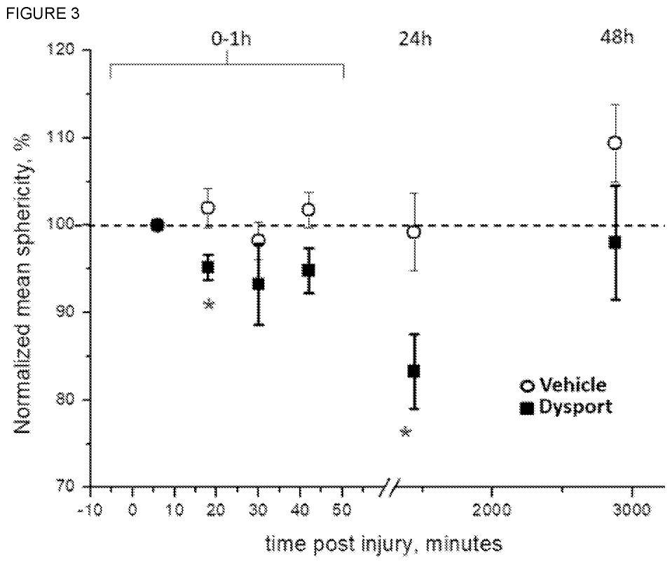

The laser-injury induced changes in the density and morphology of the microglia in the Area 1 were analysed (see FIGS. 2 and 3 for analysis results). First, a statistically significant increase was observed in the density of microglia after focal laser injury in Dysport-treated mice but not in the Vehicle-treated mice (FIG. 2; p<0.05 vs baseline for all timepoints staring from 18 minutes in Dysport group; p>0.05 vs baseline for all timepoints in Vehicle group). During the first post-injury hour the difference in the microglia density in the lesion area between the groups reached the level of statistical significance (p<0.05 between the treatment groups at 18 and 42 minutes). These observations suggest the increased migration of microglia into the lesion area in Dysport-treated mice during the first post-lesion hour.

Next, the changes in the morphology of the microglia in the lesion-containing Area 1 were analysed to find the signs of microglial activation. For this, three parameters were analysed: mean volume, mean surface area and mean sphericity index (FIG. 3). For the first two parameters the transformation of microglia to activated state is associated with increase in the values (microglia somata swell and become enlarged), while for sphericity index the activation is accompanied by a decrease in the values (more elongated shape).

It was found that both for mean volume and mean surface area, there was a gradual increase in the values after the laser injury, characteristic of more activated microglia shape. Interestingly, the difference between the treatment groups was observed for the mean sphericity of the cells (see FIG. 3). Summarising, these data suggests that Dysport affects the post-lesion change in density and sphericity of microglia but not in the volume and cell surface area. In other words, Dysport may mostly enhance migration of the microglia cells towards the lesion site but not the activation-related morphological changes.

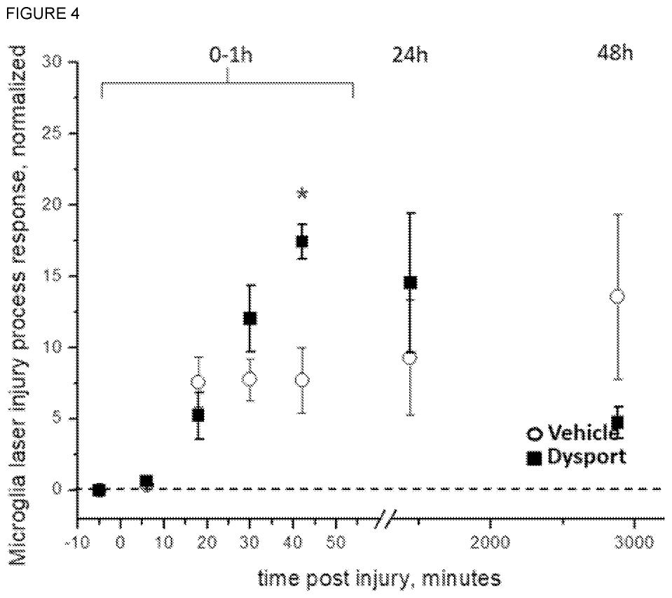

Next, the effects of Dysport injection on laser-induced microglia process extension and scar formation was investigated (see FIG. 4). In this analysis, the immediate vicinity (50 μm) of the lesion spot was analysed. A fast brightening of the scar fluorescence was observed which peaked around 40 minutes after laser injury induction (FIG. 4). A single run of Grubb's outlier test was used to remove any outliers. A statistically significant difference (Dysport treated vs Vehicle-only controls) was observed at the timepoint of 42 minutes post-injury induction (FIG. 7, p-value of 0.0303 in Mann-Whitney test). At this timepoint scar fluorescence was stronger in Dysport-treated mice, which is consistent with the notion that Dysport enhances microglial process extension toward the lesion site.

Finally, the density and morphology of microglia in Area 2 was analysed, where laser injury was not induced (e.g. Area 2 providing a control area). Briefly, no statistically significant differences were observed between the treatment groups in terms of density. In this Area 2, imaging took place at 4 timepoints: at Day 0 before and after intracortical injection, at Day 1 (24 hours) and at Day 2 (48 hours). Decline in sphericity index at Area 2 was observed, suggestive of microglia activation and migration away e.g. toward injury at Area 1.

In summary, no significant effects were observed of Dysport injection on microglia in the Area 2 which remained intact in terms of laser injury. This demonstrates that the effect of Dysport occurs responsive to a site of injury.

Summary of Results

Microglia are a major player in regulating neuroprotection and neuronal cell death. Here, the inventor investigated the influence of clostridial neurotoxin administration on injury-induced microglia activation.

Adult, female CX3CR1-GFP heterozygous C57BL/6JCRL mice (n=24) were anesthetized before being implanted with chronic cranial glass windows (equipped with injection access ports) suitable for repeated in vivo two-photon imaging. After 7 days, 15 animals, selected for imaging studies, were anesthetized prior to injection of aboBoNT-A (0.5 U dissolved in 2 μL) or its vehicle (saline) in somatosensory cortex ˜1 h before the adjacent area received a μm-size infrared laser irradiation injury to induce microglia activation. Imaging sessions were performed at baseline, after aboBoNT-A injection (before irradiation) and after laser injury at 10-min intervals during the first 40 min and again 24- and 48 h post-injury.

Intracortical injections of aboBoNT-A 0.5 U in mice were safe and did not affect behavior or body weight growth, with no substantially observable effect on microglia before laser injury. After laser injury, aboBoNT-A induced between 15 and 50% increases (p<0.05) in microglia density 10-40 min post-injury, a decrease in soma sphericity and enhancement of processes extension, compared with the Vehicle-injected group. The inventor observed no significant differences in microglial density between Vehicle and aboBoNT-A groups in a ‘control’ area of of the same brain (somatosensory cortex) which was left intact i.e. unaffected by laser injury.

In summary, while microglia morphology and density was not observably changed by aboBoNT-A in the absence of the laser injury, aboBoNT-A significantly enhanced (promoted) laser injury-induced microglial cell migration and process extension. Thus, aboBoNT-A can be employed to mediate upregulation of microglial activation.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A clostridial neurotoxin for use in a method of treating a brain disorder in a patient by promoting a neuroimmune response.

2. A clostridial neurotoxin for use in a method of treating brain tissue damage in a patient by promoting a neuroimmune response.

3. The clostridial neurotoxin for use according to claim 1 or claim 2, wherein the clostridial neurotoxin promotes microglia cell activity in the brain.

4. The clostridial neurotoxin for use according to any one of the preceding claims, wherein the clostridial neurotoxin promotes microglia cell migration to a site of brain tissue damage.

5. The clostridial neurotoxin for use according to any one of the preceding claims, wherein the clostridial neurotoxin promotes microglia cell phagocytic activity at a site of brain tissue damage.

6. The clostridial neurotoxin for use according to any one of the preceding claims, wherein the clostridial neurotoxin promotes microglia cell migration to a site of infection.

7. The clostridial neurotoxin for use according to any one of the preceding claims, wherein the clostridial neurotoxin promotes microglia cell phagocytic activity at a site of infection.

8. The clostridial neurotoxin for use according to any one of the preceding claims, wherein the neuroimmune response is promoted in ≤60 minutes following administration of the clostridial neurotoxin.

9. The clostridial neurotoxin for use according to any one of the preceding claims, wherein the neuroimmune response is promoted in ≤10 minutes following administration of the clostridial neurotoxin.

10. The clostridial neurotoxin for use according to any one of the preceding claims, wherein the neuroimmune response is promoted for ≤48 hours following the occurrence of brain tissue damage.

11. The clostridial neurotoxin for use according to any one of the preceding claims, wherein the neuroimmune response is promoted for ≤24 hours following the occurrence of brain tissue damage.

12. The clostridial neurotoxin for use according to any one of the preceding claims, wherein the clostridial neurotoxin is administered by local administration to the patient's head.

13. The clostridial neurotoxin for use according to any one of the preceding claims, wherein the clostridial neurotoxin is administered by local administration to the patient's head by subcutaneous or intracranial injection.

14. The clostridial neurotoxin for use according to any one of the preceding claims, wherein the clostridial neurotoxin is administered by intracranial injection.

15. The clostridial neurotoxin for use according to any one of the preceding claims, wherein the clostridial neurotoxin is administered before brain tissue damage occurs.

16. The clostridial neurotoxin for use according to any one of the preceding claims, wherein the clostridial neurotoxin is administered ≤4 days before brain tissue damage occurs.

17. The clostridial neurotoxin for use according to any one of the preceding claims, wherein the clostridial neurotoxin is administered between 1-3 days before brain tissue damage occurs.

18. The clostridial neurotoxin for use according to any one of the preceding claims, wherein the clostridial neurotoxin is administered ≤24 hours following the occurrence of brain tissue damage.

19. The clostridial neurotoxin for use according to any one of the preceding claims, wherein the clostridial neurotoxin is administered ≤2 hours following the occurrence of brain tissue damage.

20. The clostridial neurotoxin for use according to any one of the preceding claims, wherein the clostridial neurotoxin is administered ≤10 minutes following the occurrence of brain tissue damage.

21. The clostridial neurotoxin for use according to any one of the preceding claims, wherein the brain disorder is caused by traumatic brain injury, cancer, infectious disease, stroke, a neurodegenerative disorder, brain aneurysm, multiple sclerosis, anoxic injury, toxic injury and/or metabolic injury.

22. The clostridial neurotoxin for use according to any one of the preceding claims, wherein the brain disorder is caused by a non-traumatic brain injury.

23. The clostridial neurotoxin for use according to any one of the preceding claims, wherein the brain disorder is caused by cancer, infectious disease, and/or stroke.

24. The clostridial neurotoxin for use according to any one of the preceding claims, wherein the brain disorder is caused by an infectious disease.

25. The clostridial neurotoxin for use according to any one of the preceding claims, wherein the brain disorder is caused by an infectious disease selected from encephalitis, meningitis, and a brain abscess.

26. The clostridial neurotoxin for use according to any one of the preceding claims, wherein the brain disorder is caused by an infectious disease selected from encephalitis, and meningitis.

27. The clostridial neurotoxin for use according to any one of the preceding claims, wherein the brain disorder is caused by a stroke.

28. The clostridial neurotoxin for use according to any one of the preceding claims, wherein the brain disorder is caused by a neurodegenerative disorder selected from: Alzheimer's disease, Parkinson's disease, Parkinson's disease related disorders, motor neuron disease (e.g. amyotrophic lateral sclerosis), prion disease, Huntington's disease, spinocerebellar ataxia, ataxia, Hallervorden-Spatz disease, and frontotemporal lobar degeneration.

29. The clostridial neurotoxin for use according to any one of the preceding claims, wherein the clostridial neurotoxin is administered subcutaneously, intracranially, intrathecally or intranasally.

30. The clostridial neurotoxin for use according to any one of the preceding claims, wherein the clostridial neurotoxin is a botulinum neurotoxin (BoNT).

31. The clostridial neurotoxin for use according to any one of the preceding claims, wherein the clostridial neurotoxin is botulinum neurotoxin serotype A (BoNT/A).

32. The clostridial neurotoxin for use according to any one of the preceding claims, wherein the clostridial neurotoxin is a chimeric neurotoxin.


SEQUENCE LISTING (76 sequences)

SEQ ID NO: 1 - DNA, 3888 bp, Artificial Sequence
  Nucleotide Sequence of rBoNT/A(0)
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatcaactgatctacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, Artificial Sequence
  Polypeptide Sequence of rBoNT/A(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 2634 bp, Artificial Sequence
  Nucleotide Sequence of rLHN/A
atggagttcgttaacaaacagttcaactataaagacccagttaacggtgttgacattgcttacatcaaaatcccgaacgctggccagatgcagccggtaaaggcattcaaaatccacaacaaaatctgggttatcccggaacgtgatacctttactaacccggaagaaggtgacctgaacccgccaccggaagcgaaacaggtgccggtatcttactatgactccacctacctgtctaccgataacgaaaaggacaactacctgaaaggtgttactaaactgttcgagcgtatttactccaccgacctgggccgtatgctgctgactagcatcgttcgcggtatcccgttctggggcggttctaccatcgataccgaactgaaagtaatcgacactaactgcatcaacgttattcagccggacggttcctatcgttccgaagaactgaacctggtgatcatcggcccgtctgctgatatcatccagttcgagtgtaagagctttggtcacgaagttctgaacctcacccgtaacggctacggttccactcagtacatccgtttctctccggacttcaccttcggttttgaagaatccctggaagtagacacgaacccactgctgggcgctggtaaattcgcaactgatcctgcggttaccctggctcacgaactgattcatgcaggccaccgcctgtacggtatcgccatcaatccgaaccgtgtcttcaaagttaacaccaacgcgtattacgagatgtccggtctggaagttagcttcgaagaactgcgtacttttggcggtcacgacgctaaattcatcgactctctgcaagaaaacgagttccgtctgtactactataacaagttcaaagatatcgcatccaccctgaacaaagcgaaatccatcgtgggtaccactgcttctctccagtacatgaagaacgtttttaaagaaaaatacctgctcagcgaagacacctccggcaaattctctgtagacaagttgaaattcgataaactttacaaaatgctgactgaaatttacaccgaagacaacttcgttaagttctttaaagttctgaaccgcaaaacctatctgaacttcgacaaggcagtattcaaaatcaacatcgtgccgaaagttaactacactatctacgatggtttcaacctgcgtaacaccaacctggctgctaattttaacggccagaacacggaaatcaacaacatgaacttcacaaaactgaaaaacttcactggtctgttcgagttttacaagctgctgtgcgtcgacggcatcattacctccaaaactaaatctgacgatgacgataaaaacaaagcgctgaacctgcagtgtatcaaggttaacaactgggatttattcttcagcccgagtgaagacaacttcaccaacgacctgaacaaaggtgaagaaatcacctcagatactaacatcgaagcagccgaagaaaacatctcgctggacctgatccagcagtactacctgacctttaatttcgacaacgagccggaaaacatttctatcgaaaacctgagctctgatatcatcggccagctggaactgatgccgaacatcgaacgtttcccaaacggtaaaaagtacgagctggacaaatataccatgttccactacctgcgcgcgcaggaatttgaacacggcaaatcccgtatcgcactgactaactccgttaacgaagctctgctcaacccgtcccgtgtatacaccttcttctctagcgactacgtgaaaaaggtcaacaaagcgactgaagctgcaatgttcttgggttgggttgaacagcttgtttatgattttaccgacgagacgtccgaagtatctactaccgacaaaattgcggatatcactatcatcatcccgtacatcggtccggctctgaacattggcaacatgctgtacaaagacgacttcgttggcgcactgatcttctccggtgcggtgatcctgctggagttcatcccggaaatcgccatcccggtactgggcacctttgctctggtttcttacattgcaaacaaggttctgactgtacaaaccatcgacaacgcgctgagcaaacgtaacgaaaaatgggatgaagtttacaaatatatcgtgaccaactggctggctaaggttaatactcagatcgacctcatccgcaaaaaaatgaaagaagcactggaaaaccaggcggaagctaccaaggcaatcattaactaccagtacaaccagtacaccgaggaagaaaaaaacaacatcaacttcaacatcgacgatctgtcctctaaactgaacgaatccatcaacaaagctatgatcaacatcaacaagttcctgaaccagtgctctgtaagctatctgatgaactccatgatcccgtacggtgttaaacgtctggaggacttcgatgcgtctctgaaagacgccctgctgaaatacatttacgacaaccgtggcactctgatcggtcaggttgatcgtctgaaggacaaagtgaacaataccttatcgaccgacatcccttttcagctcagtaaatatgtcgataaccaacgccttttgtccactctagaagcacaccatcatcaccaccatcaccatcaccat

SEQ ID NO: 4 - protein, 878 aa, Artificial Sequence
  Polypeptide Sequence of rLHN/A
MEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSDDDDKNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEAHHHHHHHHHH

SEQ ID NO: 5 - DNA, 1329 bp, Artificial Sequence
  Nucleotide Sequence of rL/A
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgggtctagaagcacaccatcatcaccaccatcaccatcaccat

SEQ ID NO: 6 - protein, 443 aa, Artificial Sequence
  Polypeptide Sequence of rL/A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLGLEAHHHHHHHHHH

SEQ ID NO: 7 - DNA, 1299 bp, Artificial Sequence
  Nucleotide Sequence of rHC/A
atgcatcatcaccatcaccacaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 8 - protein, 433 aa, Artificial Sequence
  Polypeptide Sequence of rHC/A
MHHHHHHKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 9 - DNA, 3873 bp, Artificial Sequence
  Nucleotide Sequence of rBoNT/B(0)
atgccggtgacgattaacaacttcaactacaacgacccgattgacaacaacaacattatcatgatggaaccgccgtttgcacgcggcacgggccgttattacaaagcgtttaaaatcaccgatcgtatttggattatcccggaacgctacacgtttggttataaaccggaagacttcaacaaaagctctggcatcttcaaccgtgatgtttgcgaatactacgatccggactacctgaacaccaacgataagaaaaacatttttctgcaaacgatgatcaaactgttcaatcgcattaaaagcaaaccgctgggtgaaaaactgctggaaatgattatcaatggcattccgtatctgggtgatcgtcgcgtgccgctggaagaatttaacaccaatatcgcgagtgttacggtcaacaaactgatttccaatccgggtgaagtcgaacgtaaaaaaggcatcttcgccaacctgatcatcttcggcccgggtccggtgctgaacgaaaatgaaaccattgatatcggtattcagaaccattttgcctcacgcgaaggcttcggcggtattatgcaaatgaaattttgcccggaatatgtgtcggttttcaacaatgttcaggaaaacaaaggtgcaagcatctttaatcgtcgcggctatttctctgatccggctctgatcctgatgcaccaactgatttatgtgctgcacggcctgtatggtatcaaagtggatgacctgccgatcgttccgaacgagaaaaaatttttcatgcagagcaccgacgcaattcaagctgaagaactgtatacgtttggcggtcaggacccgtctattatcaccccgagcaccgacaaaagcatctacgataaagtgctgcaaaactttcgtggcattgttgaccgcctgaataaagtcctggtgtgtatctctgatccgaacatcaacatcaacatctacaaaaacaaattcaaagacaaatacaaattcgttgaagattctgaaggcaaatatagtattgacgtcgaatcctttgataaactgtacaaaagtctgatgttcggtttcaccgaaacgaacatcgcggaaaactacaaaatcaaaacccgcgcctcctatttcagcgactctctgccgccggttaaaatcaaaaatctgctggataacgaaatttatacgatcgaagaaggtttcaacatcagcgataaagacatggaaaaagaataccgtggccagaataaagcaatcaacaaacaggcgtatgaagaaattagtaaagaacatctggcggtctacaaaattcagatgtgcaaatccgtgaaagccccgggtatttgtatcgatgttgacaatgaagacctgtttttcatcgccgataaaaacagtttttccgatgacctgtcaaaaaatgaacgcatcgaatacaacacccaatcgaactacatcgaaaacgatttcccgatcaacgaactgattctggatacggacctgattagtaaaatcgaactgccgtcagaaaacaccgaatcgctgacggactttaatgttgatgtcccggtgtatgaaaaacagccggcaattaagaaaatttttaccgatgaaaacacgatcttccagtacctgtacagccaaacctttccgctggacattcgcgatatctctctgacgagttcctttgatgacgcactgctgttcagcaacaaagtgtactcctttttctcaatggattacatcaaaaccgctaacaaagtggttgaagcgggcctgtttgccggttgggtgaaacagatcgttaacgatttcgtcatcgaagccaacaaaagtaacacgatggataaaattgctgatatctccctgattgtcccgtatattggcctggcactgaatgtgggtaacgaaacggcgaaaggcaattttgaaaacgccttcgaaattgcaggcgcttcaatcctgctggaatttattccggaactgctgatcccggtcgtgggtgcgttcctgctggaatcttacatcgacaacaaaaacaaaatcatcaaaaccattgataacgcgctgacgaaacgtaacgaaaaatggtcagatatgtacggcctgattgttgcccagtggctgagcaccgtcaacacgcaattttacaccatcaaagaaggtatgtacaaagcgctgaattatcaggcgcaagccctggaagaaatcatcaaataccgctacaacatctacagcgaaaaagaaaaatctaacatcaacatcgactttaatgatatcaacagcaaactgaacgaaggtatcaaccaggcaatcgataacatcaacaacttcatcaacggctgctcagtgtcgtatctgatgaagaaaatgatcccgctggctgttgaaaaactgctggattttgacaacaccctgaagaaaaacctgctgaactacatcgatgaaaacaaactgtacctgatcggctcagccgaatacgaaaaatcgaaagtgaacaaatacctgaaaaccatcatgccgtttgacctgagtatttacaccaacgatacgatcctgatcgaaatgttcaacaaatacaactccgaaattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaagagaaactgttcctggccccgattagcgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaa

SEQ ID NO: 10 - protein, 1291 aa, Artificial Sequence
  Polypeptide Sequence of rBoNT/B(0)
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHQLIYVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 11 - DNA, 3873 bp, Artificial Sequence
  Nucleotide Sequence of rBoNT/C(0)
atgccgatcacgattaataatttcaactatagcgatccggtggacaataagaatattctgtatctggatactcatctgaatacgctggctaacgaaccggagaaagcgttccgcatcacaggcaacatctgggttattcccgatcgcttttcacgcaacagcaaccctaatctgaacaaacctcctcgtgtcaccagtcctaaatccggttattacgacccaaactatctgagtacggatagcgataaagatccctttctgaaagagatcattaagctgttcaaacgcattaactctcgcgaaattggggaagagctgatctatcggctttcgacagatatcccgttcccaggtaacaataataccccgattaatactttcgactttgatgttgatttcaattctgtggatgtgaaaacgcgtcaaggcaataattgggtgaaaactggtagcattaacccgagtgtaattatcacaggtccccgtgagaacatcatcgacccggaaacctctaccttcaagctgacgaacaacacgtttgctgcacaggaagggtttggtgccctgtcaatcatttccatctcaccgcgtttcatgttaacctactccaatgccacaaatgatgttggcgaaggacgttttagcaaatcagaattttgcatggacccaattctcattctgatgggcacgctgaacaatgcgatgcacaacttgtatggcattgctattccaaacgatcaaaccattagctccgttaccagtaatatcttctatagccagtataatgtcaaattggagtatgccgaaatttacgcctttggaggcccgaccattgacctgattccgaaatctgcacgcaaatacttcgaagaaaaggcgttagattactatcgcagcatcgcgaaacgcctgaactcgattaccacggccaatccgtcgtcgttcaacaaatacattggtgaatataaacagaaactgattcgcaaatatcggtttgtcgtagaaagctctggtgaagtgactgtaaaccgcaacaaatttgtcgaactctacaacgagttgacccaaatctttaccgagtttaactacgcaaagatctataacgtacagaaccgcaagatttatcttagcaatgtatacacaccggttactgcgaacatcttagacgacaatgtgtatgatattcagaatggctttaacatcccgaaatcaaatctgaacgttctgtttatgggccagaacctgagtcgtaatccagcactgcgtaaagtgaacccggaaaatatgctctacttgtttaccaaattttgccacaaagcgattgatggccgctctctctataacaaaacgctggattgtcgtgagttacttgtgaagaacactgatttaccgttcattggggatatctccgacgtgaaaaccgatatcttcctgcgcaaagacattaatgaagaaacggaagtcatctattaccccgacaatgtgagcgttgatcaggtcattttatcgaagaacacctccgaacatggtcagttggatttgctgtaccctagcattgactcggagagtgaaatccttccgggcgaaaatcaagtgttttacgacaaccgtacccaaaatgttgattatttgaattcttattactacctggaatctcagaaattgagcgacaatgtggaagatttcacgttcacacgctccattgaggaagcgctggataatagcgcgaaagtgtatacgtatttccctaccttggcgaataaagtaaatgctggtgtccagggaggcttatttctgatgtgggcgaatgatgtggtagaagattttacgaccaatattttgcgtaaggacaccttagataaaattagcgatgttagcgccatcatcccctatattggcccagcactgaatatctcgaactctgtgcgtcgcggaaacttcaccgaagcatttgcggtgaccggggttactattctgttggaagcctttccggagtttactattccggcgctgggtgcgtttgtgatttattcgaaagtacaagaacgcaatgaaattatcaaaaccatcgataattgcctggaacaacgcattaaacgctggaaggattcttatgaatggatgatgggcacctggttatcccgtattatcacacagtttaacaacatctcgtatcagatgtacgattcactgaactaccaagcaggggcgatcaaagccaagatcgacttagaatacaagaaatattcaggtagcgataaagagaatattaaaagccaggttgaaaacctgaagaactctctggatgtcaaaatttcagaggctatgaacaacattaacaaatttatccgcgaatgtagcgtcacgtatctgtttaaaaacatgctcccgaaagtgattgatgagctcaacgagtttgatcgcaacacaaaggccaaactgattaacctgattgatagtcacaatattattttagtcggtgaagttgacaagctgaaggctaaggtcaataacagctttcagaacactattccgtttaatattttctcctatacgaacaatagtctgctgaaagacattatcaacgaatacttcaacaatattaatgacagcaaaattctgagcctgcagaatcgtaagaatacgctggtagataccagtggatataatgcggaagtctcagaagagggtgatgtacagctgaacccgatctttccgttcgactttaaactggggtctagtggtgaagatcgcggtaaagtgatcgttacccaaaacgagaacattgtgtataacagcatgtacgagagtttctcaatttctttctggattcgcatcaataaatgggtttctaatttgcctggctataccatcattgatagcgtcaaaaacaactcgggctggtcgattggcattattagcaactttctggtgtttaccctgaaacagaatgaggattcggaacagagcattaacttctcctacgacatcagcaacaatgcaccagggtataacaaatggttcttcgtaacggtgacgaacaatatgatgggcaatatgaaaatctacattaacgggaaacttatcgacaccattaaagtgaaagagcttactgggatcaattttagtaaaaccattacctttgagatcaacaaaattccggacacgggtctgattacctccgattcggataatatcaatatgtggattcgcgacttttatatcttcgccaaagaacttgatggcaaagatatcaacattttgtttaattccctgcagtataccaatgtcgttaaggactattggggcaatgatctccgctacaataaagaatactacatggttaacatcgactatctcaatcgctacatgtatgctaactcgcgtcaaattgtgtttaacacacgtcgtaacaacaacgattttaacgaaggttataaaatcattatcaaacggatccgcggcaatacgaacgatactcgtgttcgtggcggtgacattctgtatttcgacatgacgattaataataaagcgtacaatctgttcatgaagaacgaaaccatgtacgccgataaccattccactgaagatatctacgcaatcggacttcgcgaacagaccaaagacattaacgacaacatcatctttcagattcaaccgatgaataatacctactactatgcctcccagatcttcaaaagtaatttcaacggcgaaaacatttcaggcatttgctcaatcggcacttatcggttccggttaggtggtgattggtatcgtcacaactaccttgttcccacagtgaaacaaggcaactatgcatcgctcttagaaagcacatctacgcattggggttttgtgccagtcagtgaa

SEQ ID NO: 12 - protein, 1291 aa, Artificial Sequence
  Polypeptide Sequence of rBoNT/C(0)
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMGTLNNAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 13 - DNA, 3756 bp, Artificial Sequence
  Nucleotide Sequence of rBoNT/E(0)
atgccgaaaatcaactctttcaactacaacgacccggttaacgaccgtaccatcctgtatatcaaaccgggtggttgccaggagttctacaaatctttcaacatcatgaaaaacatctggatcatcccggaacgtaacgttatcggtaccaccccgcaggacttccacccgccgacctctctgaaaaacggtgactcttcttactacgacccgaactacctccagtctgacgaagaaaaagaccgtttcctgaaaatcgttaccaaaatcttcaaccgtatcaacaacaacctgtctggtggtatcctgctggaagaactgtctaaagctaacccgtacctgggtaacgacaacaccccggacaaccagttccacatcggtgacgcttctgctgttgaaatcaaattctctaacggttctcaggacatcctgctgccgaacgttatcatcatgggtgctgaaccggacctgttcgaaaccaactcttctaacatctctctgcgtaacaactacatgccgtctaaccacggtttcggttctatcgctatcgttaccttctctccggaatactctttccgtttcaacgacaacagcatgaacgagttcatccaggacccggctctgaccctgatgcaccaactgatctactctctgcacggtctgtacggtgctaaaggtatcaccaccaaatacaccatcacccagaaacagaacccgctgatcaccaacatccgtggtaccaacatcgaagagttcctgaccttcggtggtaccgacctgaacatcatcacctctgctcagtctaacgacatctacaccaacctgctggctgactacaaaaaaatcgcttctaaactgtctaaagttcaggtttctaacccgctgctgaacccgtacaaagacgttttcgaagctaaatacggtctggacaaagacgcttctggtatctactctgttaacatcaacaaattcaacgacatcttcaaaaaactgtactctttcaccgagttcgacctggcgaccaaattccaggttaaatgccgtcagacctacatcggtcagtacaaatacttcaaactgtctaacctgctgaacgactctatctacaacatctctgaaggttacaacatcaacaacctgaaagttaacttccgtggtcagaacgctaacctgaacccgcgtatcatcaccccgatcaccggtcgtggtctggttaaaaaaatcatccgtttctgcaagaatattgtaagcgttaaaggaataagaaaaagtatctgcatcgaaatcaacaacggtgaactgttcttcgttgcttctgaaaactcttacaacgacgacaacatcaacaccccgaaagaaatcgacgacaccgttacctctaacaacaactacgaaaacgacctggaccaggttatcctgaacttcaactctgaatctgctccgggtctgtctgacgaaaaactgaacctgaccatccagaacgacgcttacatcccgaaatacgactctaacggtacctctgacatcgaacagcacgacgttaacgaactgaacgttttcttctacctggacgctcagaaagttccggaaggtgaaaacaacgttaacctgacctcttctatcgacaccgctctgctggaacagccgaaaatctacaccttcttctcttctgagttcatcaacaacgttaacaaaccggttcaggctgctctgttcgtttcttggattcagcaggttctggttgacttcaccaccgaagctaaccagaaatctaccgttgacaaaatcgctgacatctctatcgttgttccgtacatcggtctggctctgaacatcggtaacgaagctcagaaaggtaacttcaaagacgctctggaactgctgggtgctggtatcctgctggagttcgaaccggaactgctgatcccgaccatcctggttttcaccatcaaatctttcctgggttcttctgacaacaaaaacaaagttatcaaagctatcaacaacgctctgaaagaacgtgacgaaaaatggaaagaagtttactctttcatcgtttctaactggatgaccaaaatcaacacccagttcaacaaacgtaaagaacagatgtaccaggctctccagaaccaggttaacgctatcaaaaccatcatcgaatctaaatacaactcttacaccctggaagaaaaaaacgaactgaccaacaaatacgacatcaaacagatcgaaaacgaactgaaccagaaagtttctatcgctatgaacaacatcgaccgtttcctgaccgaatcttctatctcttacctgatgaaactcatcaacgaagttaaaatcaacaaactgcgtgaatacgacgaaaacgttaaaacctacctgctgaactacatcatccagcacggttctatcctgggtgaatctcagcaggaactgaactctatggttaccgacaccctgaacaactctatcccgttcaaactgtcttcttacaccgacgacaaaatcctgatctcttacttcaacaaattctttaaacgcattaagagttcatcggttctgaatatgcggtacaaaaatgataaatatgtcgatacttctggatatgatagcaatatcaacattaacggcgacgtgtataaatatccgacaaataaaaaccagtttgggatatataacgacaagctgtcggaggtcaatatttctcaaaacgactatatcatttacgataataaatataaaaactttagcattagtttttgggttcgtatacctaattatgacaataaaattgtaaatgtgaataacgagtataccattataaactgtatgcgcgacaataacagtggttggaaggtatcgctgaaccataatgagattatctggaccctgcaggataatgcaggtataaaccagaaactggcttttaactatggaaacgcaaatgggatctcagattacattaataaatggatttttgttaccattacgaacgatcgcttaggcgactcaaaactttatattaatggcaatctgatagatcagaaatcaatcttaaatttgggcaatattcatgtctctgataacatcttgttcaagatcgttaattgcagttacactcgttatattggcattcgttactttaatatcttcgataaagaactggacgagacggaaatccagactctgtattcaaacgagcccaatactaatatattgaaagatttttggggtaactatcttttatatgataaagaatactatctcctgaatgtattgaagccaaacaatttcatagatagacgcaaggatagcacattaagtatcaacaatatcagatctactatactgttagcaaatcgcctctactccggtattaaagtgaagattcagcgggttaataactccagtaccaatgataatctggtccgtaagaacgatcaggtatacatcaatttcgtcgcgagcaaaactcatctcttcccgctttacgccgatacagctacgacaaacaaggaaaaaaccataaaaatttccagctccggaaacagattcaatcaagtagttgtaatgaactctgtgggtaataattgtacgatgaactttaagaataacaatgggaacaatattggacttttgggcttcaaagccgacacagtggtggcgtccacctggtattacacgcacatgcgggaccatacgaattcgaacggttgcttctggaactttatctcggaagaacacgggtggcaagaaaaa

SEQ ID NO: 14 - protein, 1252 aa, Artificial Sequence
  Polypeptide Sequence of rBoNT/E(0)
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHQLIYSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 15 - DNA, 3834 bp, Artificial Sequence
  Nucleotide Sequence of rBoNT/F(0)
atgccggtggtcatcaacagcttcaactacaacgacccagtaaacgacgacacgatcctgtatatgcaaatcccgtatgaagagaagagcaagaagtactataaggcctttgaaatcatgcgcaatgtgtggattattccggagcgtaatacgattggtactgacccaagcgacttcgatccacctgcgtctttggaaaacggctcgtccgcatattacgacccgaattacctgaccaccgatgcggagaaagatcgttatttgaaaaccaccatcaagctgttcaaacgcattaacagcaatccggcaggtgaggtcctgctgcaagagattagctacgcaaagccttatctgggtaatgagcatacgcctattaacgagtttcacccggttacccgcactaccagcgttaacatcaagtcctcgaccaacgtgaagtctagcattatcctgaacctgctggttctgggtgccggtccggacatcttcgaaaactctagctacccggtgcgtaaactgatggatagcggcggtgtttatgacccgagcaatgacggttttggcagcatcaatatcgtgacgtttagcccggagtacgagtacaccttcaatgatatcagcggtggttacaattcttctaccgagagcttcatcgccgacccggcgatcagcctggcacaccaactgatctatgcattgcatggcttgtacggtgcccgtggtgtgacgtataaagagactatcaaggttaagcaggcacctctgatgattgcggaaaagccgattcgcctggaagagttcctgaccttcggcggtcaagatttgaacatcattacctcggccatgaaagagaaaatctataacaatttgctggccaactatgaaaagattgcaacgcgcttgtctcgtgttaactccgctccgccggaatacgacattaatgagtacaaagactactttcaatggaaatatggcctggacaaaaatgcggatggttcttataccgtgaatgaaaacaaattcaatgaaatctacaagaaactgtacagcttcaccgaaatcgatctggcgaacaagttcaaagtcaaatgtcgtaatacctacttcatcaaatatggcttcctgaaagtcccgaacctgctggacgatgacatctataccgtcagcgaaggcttcaacatcggcaatctggccgtgaataatcgtggtcagaacatcaaactgaatccgaaaatcattgactccatcccagacaagggcctggttgagaaaatcgtgaagttctgcaaaagcgttattccgcgtaaaggtacgaaagcaccgcctcgcctgtgcattcgcgttaacaaccgtgagttgttctttgtggcatctgaaagcagctacaacgagaacgacatcaacacccctaaagaaattgatgataccacgaacctgaataacaattatcgcaacaatctggacgaggtgatcctggattacaattcggaaaccattccgcaaattagcaatcagacgctgaacaccctggttcaggacgatagctacgttccgcgttacgactccaatggtactagcgagattgaagaacacaacgtagtggacttgaacgttttcttttatctgcacgcccagaaggttccggagggcgaaaccaatattagcctgaccagctcgatcgacaccgcgctgtctgaggagagccaagtctacacctttttcagcagcgagtttatcaacactattaacaagccagttcatgctgcattgtttatctcttggattaaccaggtgattcgcgactttacgacggaggcgacccagaagtctaccttcgacaaaattgcagacatctccctggtcgtcccatacgtcggcctggcgttgaatattggcaatgaagttcaaaaagagaacttcaaagaagcgttcgagctgctgggtgcaggcatcctgctggagttcgtgccggaactgttgatcccgaccatcctggtgttcaccattaagagcttcattggatcctccgagaataagaacaagatcatcaaggcgatcaataacagcctgatggagcgtgaaacgaagtggaaagaaatctatagctggattgttagcaattggctgactcgtattaacacgcaattcaacaagcgtaaagagcaaatgtaccaagccctgcaaaaccaagttgacgccatcaaaacggtaattgaatacaagtacaacaattacacgagcgatgagcgcaaccgcctggaaagcgaatacaacatcaacaacattcgcgaagaattgaacaagaaagtgagcctggcgatggagaacattgagcgttttatcaccgaaagcagcatcttttacctgatgaaattgattaatgaggcgaaagtctcgaaactgcgtgagtacgacgaaggtgtgaaagagtatctgctggattacattagcgagcaccgtagcatcttgggtaactcggttcaggagctgaacgatctggtgacctctaccctgaacaatagcatcccgttcgaactgagcagctataccaatgacaagattctgattctgtatttcaataaactgtataagaagatcaaggataacagcattctggatatgcgttacgaaaacaataagtttatcgacatttctggttacggcagcaacatttccatcaatggcgatgtctacatctacagcaccaatcgcaaccagttcggcatctactctagcaaaccgagcgaagttaacatcgcacagaacaatgatattatttataacggtcgttatcaaaacttctctatcagcttttgggtccgtatcccgaagtacttcaataaagtcaatctgaataatgaatacacgatcatcgactgcattcgcaataacaacagcggttggaaaatcagcctgaattacaacaaaattatttggaccctgcaagatacggcgggtaacaatcagaaactggtgtttaactacacgcaaatgatcagcatttctgactatatcaacaagtggatctttgttaccatcaccaataatcgtctgggcaatagccgtatttacatcaacggtaacctgattgatgagaaaagcatcagcaacctgggcgatattcacgtcagcgacaacattctgttcaaaattgttggttgtaacgatacccgttacgtcggcatccgttatttcaaggttttcgatacggagctgggtaaaacggaaatcgaaacgttgtactccgatgaaccagatccgagcattctgaaggacttttggggtaactacttgctgtacaataaacgttactatctgctgaatctgttgcgcaccgacaagagcattacccaaaacagcaatttcctgaacattaatcagcaacgcggcgtataccaaaaaccgaacatcttcagcaatacgcgcctgtatactggtgttgaagtgatcattcgtaagaacggtagcaccgacattagcaacacggacaatttcgtccgtaagaatgacctggcgtacattaacgtcgtggaccgtgatgtcgagtatcgtctgtacgcagacatcagcattgcgaaaccggaaaagattatcaagctgatccgtaccagcaacagcaacaacagcctgggtcagatcattgtgatggacagcattggtaataactgcacgatgaacttccagaacaacaatggtggtaatatcggtctgctgggttttcacagcaataatctggttgcttccagctggtactacaataacattcgtaaaaacacgtctagcaatggttgtttttggagctttatcagcaaagagcacggctggcaagaaaat

SEQ ID NO: 16 - protein, 1278 aa, Artificial Sequence
  Polypeptide Sequence of rBoNT/F(0)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHQLIYALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 17 - DNA, 3942 bp, Artificial Sequence
  Nucleotide Sequence of rBoNT/A(0) (His-tagged)
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaaagctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacaccagctgatttatgccggtcatcgtctgtatggtattgccattaatccgaatcgtgtgttcaaagtgaataccaacgcctattatgaaatgagcggtctggaagtgagttttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattattaccagcaaaaccaaaagtctggataaaggctacaataaagccctgaatgatctgtgcattaaggtgaataattgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatttttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaatcaggcagaagcaaccaaagccattatcaactatcagtataaccagtacaccgaagaagagaaaaataacatcaacttcaacatcgacgatctgtccagcaaactgaacgaaagcatcaacaaagccatgattaacattaacaaatttctgaaccagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgtcaacctttaccgaatacattaagaacatcatcaacaccagcattctgaacctgcgttatgaaagcaatcatctgattgatctgagccgttatgccagcaaaatcaatataggcagcaaggttaacttcgacccgattgacaaaaatcagatacagctgtttaatctggaaagcagcaaaattgaggtgatcctgaaaaacgccattgtgtataatagcatgtacgagaatttctcgaccagcttttggattcgtatcccgaaatactttaatagcatcagcctgaacaacgagtacaccattattaactgcatggaaaacaatagcggctggaaagttagcctgaattatggcgaaattatctggaccctgcaggatacccaagaaatcaaacagcgtgtggttttcaaatacagccagatgattaatatcagcgactatatcaaccgctggatttttgtgaccattaccaataatcgcctgaataacagcaagatctatattaacggtcgtctgattgaccagaaaccgattagtaatctgggtaatattcatgcgagcaacaacatcatgtttaaactggatggttgtcgtgatacccatcgttatatttggatcaagtacttcaacctgttcgataaagagttgaacgaaaaagaaattaaagacctgtatgataaccagagcaacagcggtattctgaaggatttttggggagattatctgcagtatgacaaaccgtattatatgctgaatctgtacgacccgaataaatacgtggatgtgaataatgttggcatccgtggttatatgtacctgaaaggtccgcgtggtagcgttatgaccacaaacatttatctgaatagcagcctgtatcgcggaaccaaattcatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagtgccctggaaattccggatgttggtaatctgagccaggttgttgtgatgaaaagcaaaaatgatcagggcatcaccaacaagtgcaaaatgaatctgcaggacaataacggcaacgatattggttttattggcttccaccagttcaacaatattgcgaaactggttgcaagcaattggtataatcgtcagattgaacgtagcagtcgtaccctgggttgtagctgggaatttatccctgtggatgatggttggggtgaacgtccgctggaaaacctgtattttcaaggtgcaagtcatcatcaccatcaccaccatcattaa

SEQ ID NO: 18 - protein, 1313 aa, Artificial Sequence
  Polypeptide Sequence of rBoNT/A(0) (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLENLYFQGASHHHHHHHH

SEQ ID NO: 19 - DNA, 2649 bp, Artificial Sequence
  Nucleotide Sequence of rLHN/A (His-tagged)
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattattaccagcaaaaccaaaagtctggataaaggctacaataaagccctgaatgatctgtgcattaaggtgaataattgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatttttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaatcaggcagaagcaaccaaagccattatcaactatcagtataaccagtacaccgaagaagagaaaaataacatcaacttcaacatcgacgatctgtccagcaaactgaacgaaagcatcaacaaagccatgattaacattaacaaatttctgaaccagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgtcaaccgaaaatctgtatttccagggtgcaagtcatcatcaccatcaccaccatcattaa

SEQ ID NO: 20 - protein, 882 aa, Artificial Sequence
  Polypeptide Sequence of rLHN/A (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTENLYFQGASHHHHHHHH

SEQ ID NO: 21 - DNA, 1323 bp, Artificial Sequence
  Nucleotide Sequence of rHC/A (His-tagged)
atgcatcatcaccatcaccacgaaaatctatacttccaaggaaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctgtaa

SEQ ID NO: 22 - protein, 440 aa, Artificial Sequence
  Polypeptide Sequence of rHC/A (His-tagged)
MHHHHHHENLYFQGKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 23 - DNA, 1326 bp, Artificial Sequence
  Nucleotide Sequence of rLC/A (His-tagged)
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgtttgaagagaatctgtatttccagggtgcaagtcatcatcaccatcaccaccatcattaa

SEQ ID NO: 24 - protein, 441 aa, Artificial Sequence
  Polypeptide Sequence of rLC/A (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEENLYFQGASHHHHHHHH

SEQ ID NO: 25 - DNA, 3918 bp, Artificial Sequence
  Nucleotide Sequence of rBoNT/FA(0) (His-tagged)
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcaccagctgatttatgttctgcatggtctgtatggcacctatatcaccgaatacaaaattaccccgaatgtggttcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcaataccaaaaacagcctgtgcattaccgttaataatcgcgacctgttttttatcgcaagccaagaaagctatggcgagaataccattaacacctataaagagattgacgataccaccacactggatccgagctttgaagatattctggataaagtgatcctgaacttcaacgaacaggttattccgcagatgccgaatcgtaatgttagcaccgatattcagaaagacaactacatcccgaaatacgattataaccgcaccgacattatcgatagctatgaagttggtcgcaactacaacacctttttctatctgaatgcccagaaatttagcccgaacgaaagcaatattaccctgaccagcagctttgatacaggtctgttagaaggtagcaaagtgtatacctttttcagcagcgatttcattaacaacatcaacaaaccggttcaggccctgctgtttattgaatgggttaaacaggtgattcgcgattttaccaccgaagcaaccaaaacctcaaccgttgataaactgaaagatattagcctggtggtgccgtatattggtctggcactgaatattggtgatgagatctacaaacagcattttgcagaagcagttgaactggttggtgcaggtctgctgctggaattttcaccggaatttcttattccgacgctgctgatttttaccatcaaaggttatctgaccggtagcattcgcgataaagacaaaatcattaaaaccctggataacgccctgaatgttcgtgatcagaaatggaaagaactgtatcgttgggttgttagcaaatggctgaccaccattaatacgcagttcaacaaacgcaaagaacaaatgtacaaagccctgaaaaatcaggccaccgccattaaaaagatcatcgagaacaaatataacaactataccaccgatgaaaaaagcaagatcgatagcagctataacatcaacgaaattgaacgcaccctgaacgaaaaaatcaatctggccatgaaaaacatcgagcagtttattaccgaaagcagcattgcctatctgatcaatatcatcaacaacgaaacgatccagaaactgaaaagctatgatgacctggttcgtcgttatctgctgggttatattcgtaatcatagcagcattctgggcaatagcgttgaagaactgaattccaaagtgaacaaccatctggataatggcattccgtttgaactgagcagttataccaatgatagcctgctgatccgctacttcaataaaaactatggcgaactgaagtacaactgcattctgaacatcaaatatgagatggatcgtgacaaactggttgatagcagcggttatcgtagccgtatcaatattggtacaggcgtcaaatttagcgagatcgataaaaatcaagtgcagctgagcaatctggaatccagcaaaattgaagtcattctgaataacggcgtcatctataacagcatgtatgaaaacttttcgaccagcttttggattcgcattccgaaatactttcgcaacatcaataacgagtacaagatcatcagctgtatgcagaataatagcggttgggaagtgagcctgaattttagcaatatgaactcgaaaatcatctggaccctgcaggataccgaaggtatcaaaaaaaccgttgtgtttcagtacacccagaacattaacattagcgactatatcaaccgctggatctttgtgaccattacaaataatcgtctgagcaacagcaaaatctacattaatggtcgcctgatcaacgaagaaagcattagcgatctgggtaatatccatgccagcaacaacattatgtttaaactggatggttgccgtgatccgcatcgttatatctggattaaatactttaacctgtttgacaaagagctgaacaagaaagaaattaaagatctgtacgacaaccagagcaatagcggtattctgaaagatttctggggtgattatctgcagtatgacaaaccgtattatatgctgaatctgtatgacccgaataagtatctggatgtgaataatgttggcatccgtggctatatgtatctgaaaggtccgcgtggtcgtattgtgaccaccaacatttatctgaatagcaccctgtatatgggcaccaaattcatcattaagaaatatgccagcggcaacaaagataacattgtgcgtaataatgatcgcgtgtatattaacgtggtggtgaagaataaagaatatcgcctggcaaccaatgcaagccaggcaggcgttgaaaaaattctgagcgcagttgaaatcccggatgttggtaatctgagccaggttgttgtgatgaaaagcgaaaatgatcagggcattcgcaacaagtgtaaaatgaatctgcaagacaataacggcaacgatattggctttatcggctttcaccagtttaataacattgcaaaactggtggccagcaactggtataaccgtcagattggtaaagcaagccgtacctttggttgtagctgggaatttatcccggttgatgatggttggggtgaaagcagcctggaaaatctgtatttccagggtgccagtcatcatcaccaccatcaccatcactga

SEQ ID NO: 26 - protein, 1305 aa, Artificial Sequence
  Polypeptide Sequence of rBoNT/FA(0) (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHQLIYVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGELKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSLENLYFQGASHHHHHHHH

SEQ ID NO: 27 - DNA, 2634 bp, Artificial Sequence
  Nucleotide Sequence of rLHN/FA (His-tagged)
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcatgaactgattcatgttctgcatggtctgtatggcacctatattaccgaatacaaaattaccccgaatgtggtgcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcaataccaaaaacagcctgtgcattaccgttaataatcgcgacctgttttttatcgcaagccaagaaagctatggcgagaataccattaacacctataaagagattgacgataccaccacactggatccgagctttgaagatattctggataaagtgatcctgaacttcaacgaacaggttattccgcagatgccgaatcgtaatgttagcaccgatattcagaaagacaactacatcccgaaatacgattataaccgcaccgacattatcgatagctatgaagttggtcgcaactacaacacctttttctatctgaatgcccagaaatttagcccgaacgaaagcaatattaccctgaccagcagctttgatacaggtctgttagaaggtagcaaagtgtatacctttttcagcagcgatttcattaacaacatcaacaaaccggttcaggccctgctgtttattgaatgggttaaacaggtgattcgcgattttaccaccgaagcaaccaaaacctcaaccgttgataaactgaaagatattagcctggtggtgccgtatattggtctggcactgaatattggtgatgagatctacaaacagcattttgcagaagcagttgaactggttggtgcaggtctgctgctggaattttcaccggaatttcttattccgacgctgctgatttttaccatcaaaggttatctgaccggtagcattcgcgataaagacaaaatcattaaaaccctggataacgccctgaatgttcgtgatcagaaatggaaagaactgtatcgttgggttgttagcaaatggctgaccaccattaatacgcagttcaacaaacgcaaagaacaaatgtacaaagccctgaaaaatcaggccaccgccattaaaaagatcatcgagaacaaatataacaactataccaccgatgaaaaaagcaagatcgatagcagctataacatcaacgaaattgaacgcaccctgaacgaaaaaatcaatctggccatgaaaaacatcgagcagtttattacagaaagcagcattgcctacctgatcaatatcatcaacaacgaaaccattcagaaactgaaaagctatgatgacctggttcgtcgttatctgctgggttatattcgtaatcatagcagcattctgggcaatagcgttgaagaactgaattccaaagtgaacaaccatctggataatggcattccgtttgaactgagcagttataccaatgatagcctgctgatccgctacttcaataaaaactatggcgaagagaacctgtatttccagggtgccagtcatcatcaccaccatcaccatcactga

SEQ ID NO: 28 - protein, 877 aa, Artificial Sequence
  Polypeptide Sequence of rLHN/FA (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGEENLYFQGASHHHHHHHH

SEQ ID NO: 29 - DNA, 1341 bp, Artificial Sequence
  Nucleotide Sequence of rHC/FA (His-tagged)
atgctgaagtataactgcatcctgaacatcaaatatgagatggatcgtgataaactggttgatagcagcggttatcgtagccgtatcaatattggcaccggtgtgaaatttagcgagatcgataaaaatcaggtgcagctgagcaatctggaaagcagcaaaattgaagtgattctgaataacggcgtgatctacaatagcatgtatgaaaacttttcgaccagcttctggattcgcattccgaaatactttcgcaacatcaacaacgagtacaagattatcagctgtatgcagaataatagcggttgggaagttagcctgaatttcagcaatatgaacagcaaaatcatttggaccctgcaggataccgaaggtatcaaaaaaaccgttgtgtttcagtacacccagaacattaacatcagcgattacattaaccgctggatctttgtgaccattaccaataatcgtctgagcaacagcaagatctatattaacggtcgcctgattaacgaagagagcattagcgatctgggtaatattcatgccagcaacaacatcatgtttaaactggatggttgtcgtgatccgcatcgttatatttggatcaaatacttcaacctgtttgataaagaactgaacaaaaaagaaatcaaagacctgtatgataaccagagcaatagcggcattctgaaagatttttggggtgattatctgcagtatgacaaaccgtattacatgctgaatctgtacgatccgaacaaatatctggatgtgaataatgtgggtatccgtggctatatgtatctgaaaggtccgcgtggtcgtattgttaccaccaacatttatctgaatagcaccctgtatatgggcaccaaattcatcattaaaaagtatgccagcggcaacaaagataacattgtgcgtaataatgatcgcgtgtatatcaatgtggtggtgaagaataaagaatatcgtctggccaccaatgcaagccaggcaggcgttgaaaaaattctgagcgcagttgaaattccggatgttggtaatctgagccaggttgttgttatgaaaagcgaaaatgatcagggcattcgcaacaaatgcaaaatgaatctgcaggacaataacggcaacgatattggttttattggcttccaccagttcaacaacattgcaaaactggtggcgagcaattggtataatcgtcagattggtaaagcaagccgtacctttggttgtagctgggaatttattccggttgatgatggttggggtgaaagcagcctggaaaatctgtattttcagggtgcaagtcatcatcaccaccatcaccatcattaa

SEQ ID NO: 30 - protein, 446 aa, Artificial Sequence
  Polypeptide Sequence of rHC/FA (His-tagged)
MLKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSLENLYFQGASHHHHHHHH

SEQ ID NO: 31 - DNA, 1347 bp, Artificial Sequence
  Nucleotide Sequence of rLC/FA (His-tagged)
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcatgaactgattcatgttctgcatggtctgtatggcacctatattaccgaatacaaaattaccccgaatgtggtgcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcgaaaatctgtattttcagggtgccagtcatcatcaccaccatcaccatcactga

SEQ ID NO: 32 - protein, 448 aa, Artificial Sequence
  Polypeptide Sequence of rLC/FA (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSENLYFQGASHHHHHHHH

SEQ ID NO: 33 - DNA, 3888 bp, Artificial Sequence
  Nucleotide Sequence of rBoNT/F(0) (His-tagged)
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacaccagctgatttatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgtgattccgcgtaaaggcaccaaagcaccgcctcgtctgtgtattcgtgtgaataatcgtgaactgttttttgttgcaagcgagagcagctataacgagaatgatattaacaccccgaaagagattgacgataccaccaatctgaataacaactatcgcaacaatctggatgaagtgatcctggattataacagcgaaaccattccgcagattagcaatcagaccctgaataccctggttcaggatgatagctatgttccgcgttatgatagcaatggcaccagcgaaattgaagaacataatgtggttgatctgaacgtgttcttttatctgcatgcacagaaagtgccggaaggtgaaaccaatattagcctgaccagcagcattgataccgcactgagcgaagaaagccaggtttataccttttttagcagcgaattcatcaacaccattaacaaaccggttcatgcagcactgtttattagctggattaatcaggtgattcgcgattttaccaccgaagcaacccagaaaagcacctttgataaaattgccgatattagtctggtggtgccgtatgttggtctggcactgaatattggtaatgaagtgcagaaagagaactttaaagaagccttcgaactgttaggtgccggtattctgctggaatttgtgccggaactgctgattccgaccattctggtttttaccattaagagctttattggcagcagcgagaacaagaacaaaatcattaaagccatcaacaacagcctgatggaacgcgaaaccaaatggaaagaaatttacagctggattgtgagcaattggctgacccgtatcaatacccagtttaacaaacgcaaagaacaaatgtatcaggccctgcagaatcaggttgatgcaattaaaaccgtgatcgaatacaaatacaacaactataccagcgacgaacgtaatcgcctggaaagcgaatacaacattaataacattcgcgaagaactgaacaaaaaagtgagcctggcaatggaaaacatcgaacgttttattaccgaaagcagcatcttctacctgatgaaactgattaacgaagccaaagttagcaaactgcgcgaatatgatgaaggcgttaaagaatatctgctggactatattagcgaacatcgtagcattctgggtaatagcgttcaagagctgaatgatctggttaccagcacactgaataatagcattccgtttgaactgagcagctacaccaacgataaaatcctgatcctgtacttcaacaaactgtacaagaagatcaaggacaacagcatactggatatgcgctatgaaaacaacaagttcattgatatcagcggctatggtagcaacattagcattaatggtgatgtgtatatctacagcaccaaccgcaatcagtttggtatttatagcagcaaaccgagcgaagttaatattgcgcagaataacgatatcatctacaacggtcgctatcagaactttagcattagcttttgggttcgcattccgaaatactttaacaaggtgaacctgaacaacgagtacaccattattgattgcattcgcaataataacagcggctggaaaatcagcctgaactataacaaaattatctggaccctgcaggataccgcaggtaataatcagaaactggtgtttaactacacccagatgattagcatcagcgactatatcaacaaatggatctttgtgaccattaccaacaatcgtctgggtaacagccgcatttatatcaatggcaatctgatcgacgaaaaaagcatttcaaatctgggcgatattcacgtgagcgataacattctgttcaaaattgttggctgcaacgatacccgttatgttggtattcgttacttcaaagtgtttgatacggaactgggcaaaacggaaattgaaaccctgtatagtgatgaaccggatccgagcattctgaaagatttttggggtaattatctgctgtacaacaaacgctactatctgctgaacctgctgcgtaccgataaaagcattacacagaatagcaactttctgaacatcaatcagcagcgtggtgtttatcagaaaccgaacatttttagcaacacccgtctgtataccggtgtggaagttattattcgtaaaaacggtagcaccgatatcagcaacaccgataactttgtgcgtaaaaatgacctggcctatattaacgttgttgatcgtgatgttgagtatcgtctgtatgcggatattagcattgccaaaccggaaaagattatcaaactgatccgtaccagcaacagcaataattcactgggtcagattatcgtgatggacagcattggtaacaattgcaccatgaatttccagaacaataacggtggtaatattggcctgctgggctttcatagcaataatctggttgcaagcagctggtattacaacaacatccgtaaaaataccagcagtaatggttgcttttggagctttatcagtaaagaacatggctggcaagaaaacgagaacctgtattttcagggtgcaagtcatcatcaccatcaccaccatcattaa

SEQ ID NO: 34 - protein, 1295 aa, Artificial Sequence
  Polypeptide Sequence of rBoNT/F(0) (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHQLIYALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQENENLYFQGASHHHHHHHH

SEQ ID NO: 35 - DNA, 2649 bp, Artificial Sequence
  Nucleotide Sequence of rLHN/F (His-tagged)
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacatgaactgattcatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgtgattccgcgtaaaggcaccaaagcaccgcctcgtctgtgtattcgtgtgaataatcgtgaactgttttttgttgcaagcgagagcagctataacgagaatgatattaacaccccgaaagagattgacgataccaccaatctgaataacaactatcgcaacaatctggatgaagtgatcctggattataacagcgaaaccattccgcagattagcaatcagaccctgaataccctggttcaggatgatagctatgttccgcgttatgatagcaatggcaccagcgaaattgaagaacataatgtggttgatctgaacgtgttcttttatctgcatgcacagaaagtgccggaaggtgaaaccaatattagcctgaccagcagcattgataccgcactgagcgaagaaagccaggtttataccttttttagcagcgaattcatcaacaccattaacaaaccggttcatgcagcactgtttattagctggattaatcaggtgattcgcgattttaccaccgaagcaacccagaaaagcacctttgataaaattgccgatattagtctggtggtgccgtatgttggtctggcactgaatattggtaatgaagtgcagaaagagaactttaaagaagccttcgaactgttaggtgccggtattctgctggaatttgtgccggaactgctgattccgaccattctggtttttaccattaagagctttattggcagcagcgagaacaagaacaaaatcattaaagccatcaacaacagcctgatggaacgcgaaaccaaatggaaagaaatttacagctggattgtgagcaattggctgacccgtatcaatacccagtttaacaaacgcaaagaacaaatgtatcaggccctgcagaatcaggttgatgcaattaaaaccgtgatcgaatacaaatacaacaactataccagcgacgaacgtaatcgcctggaaagcgaatacaacattaataacattcgcgaagaactgaacaaaaaagtgagcctggcaatggaaaacatcgaacgttttattaccgaaagcagcatcttctacctgatgaaactgattaacgaagccaaagttagcaaactgcgcgaatatgatgaaggcgttaaagaatatctgctggactatattagcgaacatcgtagcattctgggtaatagcgttcaagagctgaatgatctggttaccagcacactgaataatagcattccgtttgaactgagcagctacaccaacgataaaatcctgatcctgtacttcaacaaactgtacaagaaagaaaacctgtattttcagggtgcaagccatcatcaccaccatcaccatcattaa

SEQ ID NO: 36 - protein, 882 aa, Artificial Sequence
  Polypeptide Sequence of rLHN/F (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKENLYFQGASHHHHHHHH

SEQ ID NO: 37 - DNA, 1296 bp, Artificial Sequence
  Nucleotide Sequence of rHC/F (His-tagged)
atgatcaaggataacagcattctggatatgcgctatgagaacaacaaattcattgatattagcggctatggcagcaacattagcattaatggtgatgtgtatatctacagcaccaaccgtaatcagtttggcatttatagcagcaaaccgagcgaagttaatattgcccagaacaacgatatcatctataacggtcgctatcagaacttcagcattagcttttgggttcgcattccgaaatacttcaataaggtgaacctgaacaacgagtataccatcattgattgcattcgcaataataacagcggctggaaaattagcctgaactacaacaaaattatctggaccctgcaggataccgcaggtaataatcagaaactggtgtttaactacacccagatgattagcatcagcgactatatcaacaaatggatctttgtgaccattaccaataatcgcctgggtaatagccgcatttatatcaatggtaacctgatcgatgagaaaagcattagcaatctgggtgatattcatgtgagcgataacatcctgtttaaaatcgtgggttgtaacgatacccgttatgttggtattcgctacttcaaagtgtttgataccgaactgggtaaaaccgaaattgaaaccctgtatagtgatgaaccggatccgagcattctgaaagatttttggggtaattatctgctgtacaacaaacgctactatctgctgaatctgctgcgtaccgataaatcaattacccagaatagcaacttcctgaacattaatcagcagcgtggtgtttatcagaaaccgaacatttttagcaacacccgtctgtataccggtgtggaagttattattcgtaaaaatggcagcaccgatatcagcaacaccgataactttgttcgcaaaaatgatctggcgtatatcaacgttgttgatcgtgatgttgaatatcgtctgtatgccgatattagcattgccaaaccggaaaaaatcatcaaactgatccgtaccagcaacagcaataattcactgggtcagattattgtgatggatagcattggtaataactgcaccatgaactttcagaacaataacggtggtaatattggtctgctgggctttcatagtaataatctggttgcaagcagctggtattataacaacatccgtaaaaataccagcagcaatggttgcttttggagctttattagcaaagaacatggctggcaagaaaacgagaatctgtattttcagggtgcaagtcatcatcaccaccatcaccatcattaa

SEQ ID NO: 38 - protein, 431 aa, Artificial Sequence
  Polypeptide Sequence of rHC/F (His-tagged)
MIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQENENLYFQGASHHHHHHHH

SEQ ID NO: 39 - DNA, 1347 bp, Artificial Sequence
  Nucleotide Sequence of rLC/F (His-tagged)
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacatgaactgattcatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgagaacctgtattttcagggtgcaagtcatcatcaccatcaccaccatcattaa

SEQ ID NO: 40 - protein, 448 aa, Artificial Sequence
  Polypeptide Sequence of rLC/F (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSENLYFQGASHHHHHHHH

SEQ ID NO: 41 - DNA, 1317 bp, Artificial Sequence
  Nucleotide Sequence of Cationic rHC/A (His-tagged)
atgatcatcaacaccagcattctgaacctgcgttatgaaagcaaacatctgattgatctgagccgttatgccagcaaaatcaatataggcagcaaggttaacttcgacccgattgacaaaaatcagatacagctgtttaatctggaaagcagcaaaattgaggtgatcctgaaaaaagcgatcgtgtataatagcatgtacgagaatttttcgaccagcttttggattcgcatcccgaaatactttaacaagattagcctgaacaacgagtataccatcattaactgcatggaaaacaatagcggttggaaagtcagcctgaattatggcgaaattatctggaccctgcaggataccaaagaaatcaaacagcgtgtggtgttcaaatacagccagatgattaatatcagcgactatatcaaccgctggatttttgtgaccattaccaataatcggctgaacaagagcaagatctatattaacggtcgtctgattgaccagaaaccgattagtaatctgggtaatattcatgcgagcaacaaaatcatgtttaaactggatggttgccgtgatacccatcgttatatttggatcaaatacttcaacctgttcgataaagagttgaacgaaaaagaaattaaagacctgtacgataaccagagcaatagcggcatactgaaagatttttggggagattatctgcagtatgacaaaccgtattatatgctgaatctgtacgacccgaataaatacgtggatgttaataatgtgggcatccgtggttatatgtacctgaaaggtccgcgtggtagcgttatgaccacaaacatttatctgaatagcagcctgtatcgcggaaccaaattcatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagtgccctggaaattccggatgttggtaatctgagccaggttgttgtgatgaaaagcaaaaacgataaaggcatcaccaacaaatgcaagatgaatctgcaggacaataacggcaatgatattggcttcattggctttcaccagtttaacaacattgcaaaactggttgcgagcaattggtataatcgtcagattgaacgtagcagtcgtaccctgggttgtagctgggaatttatccctgtggatgatggttggggtgaacgtccgctgaagcttgcggccgcactcgagcaccaccaccaccaccactga

SEQ ID NO: 42 - protein, 438 aa, Artificial Sequence
  Polypeptide Sequence of Cationic rHC/A (His-tagged)
MIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPLKLAAALEHHHHHH

SEQ ID NO: 43 - DNA, 1341 bp, Artificial Sequence
  Nucleotide Sequence of rHC/AB (His-tagged)
atgattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaaatgaaactgttcctggccccgatttacgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaaaagcttgcggccgcactcgagcaccaccaccaccaccactga

SEQ ID NO: 44 - protein, 446 aa, Artificial Sequence
  Polypeptide Sequence of rHC/AB (His-tagged)
MILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEKLAAALEHHHHHH

SEQ ID NO: 45 - DNA, 1306 bp, Artificial Sequence
  Nucleotide Sequence of rHC/A Variant Y1117V H1253K (His-tagged)
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttcaaacagttcaacaatattgctaaactggtagcgagcaattggtacaatcgtcagattgagcgcagcagccgtactttgggctgtagctgggagtttatcccggtcgatgatggttggggcgaacgtccgctgcaccatcaccatcaccatcaccatcaccatt

SEQ ID NO: 46 - protein, 435 aa, Artificial Sequence
  Polypeptide Sequence of rHC/A Variant Y1117V H1253K (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFKQFNNIAKLVASNWYNRQIERSSRTLGCSWEFIPVDDGWGERPLHHHHHHHHHH

SEQ ID NO: 47 - DNA, 1308 bp, Artificial Sequence
  Nucleotide Sequence of rHC/A Variant Y1117V F1252Y H1253K L1278F (His-tagged)
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggttacaaacagttcaacaatattgctaaactggtagcgagcaattggtacaatcgtcagattgagcgcagcagccgtacttttggctgtagctgggagtttatcccggtcgatgatggttggggcgaacgtccgctgcaccatcaccatcaccatcaccatcaccattaa

SEQ ID NO: 48 - protein, 435 aa, Artificial Sequence
  Polypeptide Sequence of rHC/A Variant Y1117V F1252Y H1253K L1278F (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGYKQFNNIAKLVASNWYNRQIERSSRTFGCSWEFIPVDDGWGERPLHHHHHHHHHH

SEQ ID NO: 49 - DNA, 1293 bp, Artificial Sequence
  Nucleotide Sequence of rHC/A Variant Y1117V F1252Y H1253K L1278H (His-tagged)
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggttacaaacagttcaacaatattgctaaactggtagcgagcaattggtacaatcgtcagattgagcgcagcagccgtactcatggctgtagctgggagtttatcccggtcgatgatggttggggcgaacgtccgctgcaccatcaccatcaccat

SEQ ID NO: 50 - protein, 431 aa, Artificial Sequence
  Polypeptide Sequence of rHC/A Variant Y1117V F1252Y H1253K L1278H (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGYKQFNNIAKLVASNWYNRQIERSSRTHGCSWEFIPVDDGWGERPLHHHHHH

SEQ ID NO: 51 - protein, 1296 aa, Clostridium botulinum [sequence omitted]

SEQ ID NO: 52 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 53 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 54 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 55 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 56 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 57 - protein, 1297 aa, Clostridium botulinum
  misc_feature (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 58 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 59 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 60 - DNA, 3888 bp, Artificial Sequence
  Nucleotide Sequence of mrBoNT/A
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaagcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 61 - protein, 1296 aa, Artificial Sequence
  Polypeptide Sequence of mrBoNT/A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 62 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 63 - protein, 1304 aa, Artificial Sequence
  Polypeptide Sequence of mrBoNT/AB
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 64 - protein, 1304 aa, Artificial Sequence
  Polypeptide Sequence of mrBoNT/AB(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 65 - protein, 1296 aa, Artificial Sequence
  Polypeptide Sequence of mrBoNT/A(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 66 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XDXXXLL

SEQ ID NO: 67 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXLL

SEQ ID NO: 68 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXIL

SEQ ID NO: 69 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXLM

SEQ ID NO: 70 - protein, 4 aa, Artificial Sequence
  Tyrosine-Based Motif
  SITE (2)..(3): Xaa can be any naturally occurring amino acid
  VARIANT (4)..(4): Xaa is a hydrophobic amino acid
YXXX

SEQ ID NO: 71 - protein, 6 aa, Clostridium botulinum
NIINTS

SEQ ID NO: 72 - protein, 6 aa, Clostridium botulinum
NIVNTS

SEQ ID NO: 73 - protein, 6 aa, Clostridium botulinum
NITNAS

SEQ ID NO: 74 - protein, 6 aa, Clostridium botulinum
NITNTS

SEQ ID NO: 75 - protein, 6 aa, Clostridium botulinum
EILNNI

SEQ ID NO: 76 - protein, 6 aa, Clostridium botulinum
DILNNI